SEQUENCE LISTING (70 sequences)

SEQ ID NO: 1 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 3 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETFAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 4 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQPGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSIRPRYYSISSSPRVDEKQASITVSVVSGEAWSGYGEYKGIASNYLANLQEGDTITCFVSTPQSGFTLPKDSETPLIMVGPGTGVAPFRSFVQARKQLKEQGQSLGEAHLYFGCRSPHEDYLYQEELENAQNEGIITLHTAFSRVPNQPKTYVQHVMEQDGKKLIELLDQGAHFYICGDGSQMAPDVEATLMKSYADVYEVSEADARLWLQQLEEKGRYAKDVWAG

SEQ ID NO: 5 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPRVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 6 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 7 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPPEGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNEPKTYVQHVMEQDGKKLIELLDQGAHFYICGDGSQMAPDVEATLMKSYAEVHQVSEADARLWLQQLEEKGRYAKDVWAG

SEQ ID NO: 8 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 9 - protein, 1049 aa, Bacillus megaterium; identical to SEQ ID NO: 2

SEQ ID NO: 10 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNADEHIEVPEDMTRLTLDTIGLCGFNYRFNSFYRDQPHPFITSMVRALDEAMNKLQRANPDDPAYDENKRQFQDDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLSAKGAENIAERGEADASDDFEGTYEEWREHMWSDLAAYFNLNIENSEDNASTLSLQFVDSAADMPLAKMHGAFSANVVASKELQQPGSARSTRHLEIELPKEASYQEGDHLGVIPRNYEGIVNRVTTRFGLDASQQIRLEAEEEKLAHLPLGKTVSVEELLQYVELQDPVTRTQLRAMAAKTVCPPHKVELEALLEKQAYKEQVLTKRLTMLELLEKYPACEMEFSEFIALLPSMRPRYYSISSSPRVDEKQASITVSVVSGEAWSGYGEYKGIASNYLAELQEGDTITCFVSTPQSGFTLPKDPETPLIMVGPGTGVAPFRGFVQARKQLKEQGQSLGEAHLYFGCRSPHEDYLYQEELENAQNEGIITLHTAFSRVPNQPKTYVQHVVEQDGKKLIELLDQGAHFYICGDGSQMAPDVEATLMKSYAEVHKVSEADARLWLQQLEEKSRYAKDVWAG

SEQ ID NO: 11 - protein, 1049 aa, Bacillus megaterium; identical to SEQ ID NO: 10

SEQ ID NO: 12 - protein, 420 aa, Mycobacterium sp.
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 13 - protein, 496 aa, Tetrahymena thermophile
MIFELILIAVALFAYFKIAKPYFSYLKYRKYGKGFYYPILGEMIEQEQDLKQHADADYSVHHALDKDPDQKLFVTNLGTKVKLRLIEPEIIKDFFSKSQYYQKDQTFIQNITRFLKNGIVFSEGNTWKESRKLFSPAFHYEYIQKLTPLINDITDTIFNLAVKNQELKNFDPIAQIQEITGRVIIASFFGEVIEGEKFQGLTIIQCLSHIINTLGNQTYSIMYFLFGSKYFELGVTEEHRKFNKFIAEFNKYLLQKIDQQIEIMSNELQTKGYIQNPCILAQLISTHKIDEITRNQLFQDFKTFYIAGMDTTGHLLGMTIYYVSQNKDIYTKLQSEIDSNTDQSAHGLIKNLPYLNAVIKETLRYYGPGNILFDRIAIKDHELAGIPIKKGTIVTPYAMSMQRNSKYYQDPHKYNPSRWLEKQSSDLHPDANIPFSAGQRKCIGEQLALLEARIILNKFIKMFDFTCPQDYKLMMNYKFLSEPVNPLPLQLTLRKQ

SEQ ID NO: 14 - protein, 394 aa, Nonomuraea dietziae
VNIDLVDQDHYATFGPPHEQMRWLREHAPVYWHEGEPGFWAVTRHEDVVHVSRHSDLFSSARRLALFNEMPEEQRELQRMMMLNQDPPEHTRRRSLVNRGFTPRTIRALEQHIRDICDDLLDQCSGEGDFVTDLAAPLPLYVICELLGAPVADRDKIFAWSNRMIGAQDPDYAASPEEGGAAAMEVYAYASELAAQRRAAPRDDIVTKLLQSDENGESLTENEFELFVLLLVVAGNETTRNAASGGMLTLFEHPDQWDRLVADPSLAATAADEIVRWVSPVNLFRRTATADLTLGGQQVKADDKVVVFYSSANRDASVFSDPEVFDIGRSPNPHIGFGGGGAHFCLGNHLAKLELRVLFEQLARRFPRMRQTGEARRLRSNFINGIKTLPVTLG

SEQ ID NO: 15 - protein, 501 aa, Homo sapiens
MWKLWRAEEGAAALGGALFLLLFALGVRQLLKQRRPMGFPPGPPGLPFIGNIYSLAASSELPHVYMRKQSQVYGEIFSLDLGGISTVVLNGYDVVKECLVHQSEIFADRPCLPLFMKMTKMGGLLNSRYGRGWVDHRRLAVNSFRYFGYGQKSFESKILEETKFFNDAIETYKGRPFDFKQLITNAVSNITNLIIFGERFTYEDTDFQHMIELFSENVELAASASVFLYNAFPWIGILPFGKHQQLFRNAAVVYDFLSRLIEKASVNRKPQLPQHFVDAYLDEMDQGKNDPSSTFSKENLIFSVGELIIAGTETTTNVLRWAILFMALYPNIQGQVQKEIDLIMGPNGKPSWDDKCKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEHLARMEMFLFFTALLQRFHLHFPHELVPDLKPRLGMTLQPQPYLICAERR

SEQ ID NO: 16 - protein, 501 aa, Macca mulatta
MWKLWGGEEGAAALGGALFLLLFALGVRQLLKLRRPMGFPPGPPGLPFIGNIYSLAASAELPHVYMRKQSQVYGEIFSLDLGGISTVVLNGYDVVKECLVHQSGIFADRPCLPLFMKMTKMGGLLNSRYGQGWVEHRRLAVNSFRYFGYGQKSFESKILEETKFFTDAIETYKGRPFDFKQLITSAVSNITNLIIFGERFTYEDTDFQHMIELFSENVELAASASVFLYNAFPWIGILPFGKHQQLFRNASVVYDFLSRLIEKASVNRKPQLPQHFVDAYFDEMDQGKNDPSSTFSKENLIFSVGELIIAGTETTTNVLRWAILFMALYPNIQGQVQKEIDLIMGPNGKPSWDDKFKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEQLARMEMFLFFTALLQRFHLHFPHELVPDLKPRLGMTLQPQPYLICAERR

SEQ ID NO: 17 - protein, 501 aa, Canis familiaris
MRGPPGAEACAAGLGAALLLLLFVLGVRQLLKQRRPAGFPPGPSGLPFIGNIYSLAASGELAHVYMRKQSRVYGEIFSLDLGGISAVVLNGYDVVKECLVHQSEIFADRPCLPLFMKMTKMGGLLNSRYGRGWVDHRKLAVNSFRCFGYGQKSFESKILEETNFFIDAIETYKGRPFDLKQLITNAVSNITNLIIFGERFTYEDTDFQHMIELFSENVELAASASVFLYNAFPWIGIIPFGKHQQLFRNAAVVYDFLSRLIEKASINRKPQSPQHFVDAYLNEMDQGKNDPSCTFSKENLIFSVGELIIAGTETTTNVLRWAILFMALYPNIQGQVQKEIDLIMGPTGKPSWDDKCKMPYTEAVLHEVLRFCNIVPLGIFHATSEDAVVRGYSIPKGTTVITNLYSVHFDEKYWRNPEIFYPERFLDSSGYFAKKEALVPFSLGKRHCLGEQLARMEMFLFFTALLQRFHLHFPHGLVPDLKPRLGMTLQPQPYLICAERR

SEQ ID NO: 18 - protein, 222 aa, Mus musculus
MGDEMDQGQNDPLSTFSKENLIFSVGELIIAGTETTTNVLRWAILFMALYPNIQGQVHKEIDLIVGHNRRPSWEYKCKMPYTEAVLHEVLRFCNIVPLGIFHATSEDAVVRGYSIPKGTTVITNLYSVHFDEKYWKDPDMFYPERFLDSNGYFTKKEALIPFSLGRRHCLGEQLARMEMFLFFTSLLQQFHLHFPHELVPNLKPRLGMTLQPQPYLICAERR

SEQ ID NO: 19 - protein, 422 aa, Bacillus halodurans
MKSNDPIPKDSPLDHTMNLMREGYEFLSHRMERFQTDLFETRVMGQKVLCIRGAEAVKLFYDPERFKRHRATPKRIQKSLFGENAIQTMDDKAHLHRKQLFLSMMKPEDEQELARLTHETWRRVAEGWKKSRPIVLFDEAKRVLCQVACEWAEVPLKSTEIDRRAEDFHAMVDAFGAVGPRHWRGRKGRRRTERWIQSIIHQVRTGSLQAREGSPLYKVSYHRELNGKLLDERMAAIELINVLRPIVAIATFISFAAIALQEHPEWQERLKNGSNEEFHMFVQEVRRYYPFAPLIGAKVRKSFTWKGVRFKKGRLVFLDMYGTNHDPKLWDEPDAFRPERFQERKDSLYDFIPQGGGDPTKGHRCPGEGITVEVMKTTMDFLVNDIDYDVPDQDISYSLSRMPTRPESGYIMANIERKYEHA

SEQ ID NO: 20 - protein, 389 aa, Streptomyces parvus
MYLGGRRGTEAVGESREPGVWEVFRYDEAVQVLGDHRTFSSDMNHFIPEEQRQLARAARGNFVGIDPPDHTQLRGLVSQAFSPRVTAALEPRIGRLAEQLLDDIVAERGDKASCDLVGEFAGPLSAIVIAELFGIPESDHTMIAEWAKALLGSRPAGELSIADEAAMQNTADLVRRAGEYLVHHITERRARPQDDLTSRLATTEVDGKRLDDEEIVGVIGMFLIAGYLPASVLTANTVMALDEHPAALAEVRSDPALLPGAIEEVLRWRPPLVRDQRLTTRDADLGGRTVPAGSMVCVWLASAHRDPFRFENPDLFDIHRNAGRHLAFGKGIHYCLGAPLARLEARIAVETLLRRFERIEIPRDESVEFHESIGVLGPVRLPTTLFARR

SEQ ID NO: 21 - protein, 414 aa, Pseudomonas putida
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 22 - protein, 515 aa, Homo sapiens
GLEALVPLAMIVAIFLLLVDLMHRHQRWAARYPPGPLPLPGLGNLLHVDFQNTPYCFDQLRRRFGDVFNLQLAWTPVVVLNGLAAVREAMVTRGEDTADRPPAPIYQVLGFGPRSQGVILSRYGPAWREQRRFSVSTLRNLGLGKKSLEQWVTEEAACLCAAFADQAGRPFRPNGLLDKAVSNVIASLTCGRRFEYDDPRFLRLLDLAQEGLKEESGFLREVLNAVPVLPHIPALAGKVLRFQKAFLTQLDELLTEHRMTWDPAQPPRDLTEAFLAKKEKAKGSPESSFNDENLRIVVGNLFLAGMVTTLTTLAWGLLLMILHLDVQRGRRVSPGCSPIVGTHVCPVRVQQEIDDVIGQVRRPEMGDQVHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVAAGQPRPSHSRVVSFLVTPSPYELCAVPR

SEQ ID NO: 23 - protein, 532 aa, Rattus norvegicus
AVLSRMRLRWALLDTRVMGHGLCPQGARAKAAIPAALRDHESTEGPGTGQDRPRLRSLAELPGPGTLRFLFQLFLRGYVLHLHELQALNKAKYGPMWTTTFGTRTNVNLASAPLLEQVMRQEGKYPIRDSMEQWKEHRDHKGLSYGIFITQGQQWYHLRHSLNQRMLKPAEAALYTDALNEVISDFIARLDQVRTESASGDQVPDVAHLLYHLALEAICYILFEKRVGCLEPSIPEDTATFIRSVGLMFKNSVYVTFLPKWSRPLLPFWKRYMNNWDNIFSFGEKMIHQKVQEIEAQLQAAGPDGVQVSGYLHFLLTKELLSPQETVGTFPELILAGVDTTSNTLTWALYHLSKNPEIQEALHKEVTGVVPFGKVPQNKDFAHMPLLKAVIKETLRLYPVVPTNSRIITEKETEINGFLFPKNTQFVLCTYVVSRDPSVFPEPESFQPHRWLRKREDDNSGIQHPFGSVPFGYGVRSCLGRRIAELEMQLLLSRLIQKYEVVLSPGMGEVKSVSRIVLVPSKKVSLRFLQRQ

SEQ ID NO: 24 - protein, 491 aa, Oryctolagus cuniculus
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTETTSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRICLGEGIARTELFLFFTTILQNFSIASPVPPEDIDLTPRESGVGNVPPSYQIRFLAR

SEQ ID NO: 25 - protein, 1061 aa, Bacillus subtilis
MKETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRHDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHIRVQSRNQDAIHADVQAVEKAASDEQKEKTEAKGTSVIGLNNRPLLVLYGSDTGTAEGVARELADTASLHGVRTETAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELAAFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 26 - protein, 1054 aa, Bacillus subtilis
MKQASAIPQPKTYGPLKNLPHLEKEQLSQSLWRIADELGPIFRFDFPGVSSVFVSGHNLVAEVCDESRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDDNIKDLLSLMLYAKDPVTGETLDDENIRYQIITFLIAGHETTSGLLSFAIYCLLTHPEKLKKAQEEADRVLTDDTPEYKQIQQLKYTRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLYGSNLGTAEGIAGELAAQGRQMGFTAETAPLDDYIGKLPEEGAVVIVTASYNGSPPDNAAGFVEWLKELEEGQLKGVSYAVFGCGNRSWASTYQRIPRLIDDMMKAKGASRLTEIGEGDAADDFESHRESWENRFWKETMDAFDINEIAQKEDRPSLSIAFLSEATETPVAKAYGAFEGVVLENRELQTADSTRSTRHIELEIPAGKTYKEGDHIGIMPKNSRELVQRVLSRFGLQSNHVIKVSGSAHMSHLPMDRPIKVADLLSSYVELQEPASRLQLRELASYTVCPPHQKELEQLVLDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPDEPETPMIMVGPGTGIAPFRGFIQARSVLKKEGSTLGEALLYFGCRRPDHDDLYREELDQAEQEGLVTIRRCYSRVENESKGYVQHLLKQDSQKLMTLIEKGAHIYVCGDGSQMAPDVEKTLRWAYETEKGASQEESADWLQKLQDQKRYIKDVWTGN

SEQ ID NO: 27 - protein, 1049 aa, Bacillus megaterium [sequence omitted]

SEQ ID NO: 28 - protein, 1065 aa, B. cereus
MEKKVSAIPQPKTYGPLGNLPLIDKDKPTLSFIKIAEEYGPIFQIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSGDQEENDLLSRMLNVPDPETGEKLDDENIRFQIITFLIAGHETTSGLLSFAIYFLLKNPDKLKKAYEEVDRVLTDPTPTYQQVMKLKYMRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFELIDYQNYQLDVKQTLTLKPGDFKIRILPRKQTISHPTVLAPTEDKLKNDEIKQHVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQNMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLALLSYSVEVQEAATRAQIREMVTFTACPPHKKELEALLEEGVYHEQILKKRISMLDLLEKYEACEIRFERFLELLPALKPRYYSISSSPLVAHNRLSITVGVVNAPAWSGEGTYEGVASNYLAQRHNKDEIICFIRTPQSNFELPKDPETPIIMVGPGTGIAPFRGFLQARRVQKQKGMNLGQAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWAGI

SEQ ID NO: 29 - protein, 1074 aa, B. licheniformis
MNKLDGIPIPKTYGPLGNLPLLDKNRVSQSLWKIADEMGPIFQFKFADAIGVFVSSHELVKEVSEESRFDKNMGKGLLKVREFSGDGLFTSWTEEPNWRKAHNILLPSFSQKAMKGYHPMMQDIAVQLIQKWSRLNQDESIDVPDDMTRLTLDTIGLCGFNYRFNSFYREGQHPFIESMVRGLSEAMRQTKRFPLQDKLMIQTKRRFNSDVESMFSLVDRIIADRKQAESESGNDLLSLMLHAKDPETGEKLDDENIRYQIITFLIAGHETTSGLLSFAIYLLLKHPDKLKKAYEEADRVLTDPVPSYKQVQQLKYIRMILNESIRLWPTAPAFSLYAKEETVIGGKYLIPKGQSVTVLIPKLHRDQSVWGEDAEAFRPERFEQMDSIPAHAYKPFGNGQRACIGMQFALHEATLVLGMILQYFDLEDHANYQLKIKESLTLKPDGFTIRVRPRKKEAMTAMPGAQPEENGRQEERPSAPAAENTHGTPLLVLYGSNLGTAEEIAKELAEEAREQGFHSRTAELDQYAGAIPAEGAVIIVTASYNGNPPDCAKEFVNWLEHDQTDDLRGVKYAVFGCGNRSWASTYQRIPRLIDSVLEKKGAQRLHKLGEGDAGDDFEGQFESWKYDLWPLLRTEFSLAEPEPNQTETDRQALSVEFVNAPAASPLAKAYQVFTAKISANRELQCEKSGRSTRHIEISLPEGAAYQEGDHLGVLPQNSEVLIGRVFQRFGLNGNEQILISGRNQASHLPLERPVHVKDLFQHCVELQEPATRAQIRELAAHTVCPPHQRELEDLLKDDVYKDQVLNKRLTMLDLLEQYPACELPFARFLALLPPLKPRYYSISSSPQLNPRQTSITVSVVSGPALSGRGHYKGVASNYLAGLEPGDAISCFIREPQSGFRLPEDPETPVIMVGPGTGIAPYRGFLQARRIQRDAGVKLGEAHLYFGCRRPNEDFLYRDELEQAEKDGIVHLHTAFSRLEGRPKTYVQDLLREDAALLIHLLNEGGRLYVCGDGSRMAPAVEQALCEAYRIVQGASREESQSWLSALLEEGRYAKDVWDGGVSQHNVKADCIART

SEQ ID NO: 30 - protein, 1065 aa, B. thuringiensis serovar konkukian
  VARIANT (1)..(1065): serovar konkukian str.97-27
MDKKVSAIPQPKTYGPLGNLPLIDKDKPTLSFIKLAEEYGPIFQIQTLSDTIIVVSGHELVAEVCDETRFDKSIEGALAKVRAFAGDGLFTSETDEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSENQEENDLLSRMLNVQDPETGEKLDDENIRFQIITFLIAGHETTSGLLSFAIYFLLKNPDKLKKAYEEVDRVLTDSTPTYQQVMKLKYIRMILNESLRLWPTAPAFSLYAKEDTVIGGKYPIKKGEDRISVLIPQLHRDKDAWGDDVEEFQPERFEELDKVPHHAYKPFGNGQRACIGMQFALHEATLVMGMLLQHFEFIDYEDYQLDVKQTLTLKPGDFKIRIVPRNQTISHTTVLAPTEEKLKKHEIKKQVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSTRGEADASGDFEEQLEQWKQSMWSDAMKAFGLELNKNMEKERSTLSLQFVSRLGGSPLARTYEAVYASILENRELQSSSSERSTRHIEISLPEGATYKEGDHLGVLPINNEKNVNRILKRFGLNGKDQVILSASGRSVNHIPLDSPVRLYDLLSYSVEVQEAATRAQIREMVTFTACPPHKKELESLLEDGVYQEQILKKRISMLDLLEKYEACEIRFERFLELLPALKPRYYSISSSPLVAQDRLSITVGVVNAPAWSGEGTYEGVASNYLAQRHNKDEIICFIRTPQSNFQLPENPETPIIMVGPGTGIAPFRGFLQARRVQKQKGMKVGEAHLYFGCRHPEKDYLYRTELENDERDGLISLHTAFSRLEGHPKTYVQHVIKEDRIHLISLLDNGAHLYICGDGSKMAPDVEDTLCQAYQEIHEVSEQEARNWLDRLQEEGRYGKDVWAGI

SEQ ID NO: 31 - protein, 1064 aa, R. metallidurans
MSTATPAAALEPIPRDPGWPIFGNLFQITPGEVGQHLLARSRHHDGIFELDFAGKRVPFVSSVALASELCDATRFRKIIGPPLSYLRDMAGDGLFTAHSDEPNWGCAHRILMPAFSQRAMKAYFDVMLRVANRLVDKWDRQGPDADIAVADDMTRLTLDTIALAGFGYDFASFASDELDPFVMAMVGALGEAMQKLTRLPIQDRFMGRAHRQAAEDIAYMRNLVDDVIRQRRVSPTSGMDLLNLMLEARDPETDRRLDDANIRNQVITFLIAGHETTSGLLTFALYELLRNPGVLAQAYAEVDTVLPGDALPVYADLARMPVLDRVLKETLRLWPTAPAFAVAPFDDVVLGGRYRLRKDRRISVVLTALHRDPKVWANPERFDIDRFLPENEAKLPAHAYMPFGQGERACIGRQFALTEAKLALALMLRNFAFQDPHDYQFRLKETLTIKPDQFVLRVRRRRPHERFVTRQASQAVADAAQTDVRGHGQAMTVLCASSLGTARELAEQIHAGAIAAGFDAKLADLDDAVGVLPTSGLVVVVAATYNGRAPDSARKFEAMLDADDASGYRANGMRLALLGCGNSQWATYQAFPRRVFDFFITAGAVPLLPRGEADGNGDFDQAAERWLAQLWQALQADGAGTGGLGVDVQVRSMAAIRAETLPAGTQAFTVLSNDELVGDPSGLWDFSIEAPRTSTRDIRLQLPPGITYRTGDHIAVWPQNDAQLVSELCERLDLDPDAQATISAPHGMGRGLPIDQALPVRQLLTHFIELQDVVSRQTLRALAQATRCPFTKQSIEQLASDDAEHGYATKVVARRLGILDVLVEHPAIALTLQELLACTVPMRPRLYSIASSPLVSPDVATLLVGTVCAPALSGRGQFRGVASTWLQHLPPGARVSASIRTPNPPFAPDPDPAAPMLLIGPGTGIAPFRGFLEERALRKMAGNAVTPAQLYFGCRHPQHDWLYREDIERWAGQGVVEVHPAYSVVPDAPRYVQDLLWQRREQVWAQVRDGATIYVCGDGRRMAPAVRQTLIEIGMAQGGMTDKAASDWFGGLVAQGRYRQDVFN

SEQ ID NO: 32 - protein, 1120 aa, A. fumigatus
MSESKTVPIPGPRGVPLLGNIYDIEQEVPLRSINLMADQYGPIYRLTTFGWSRVFVSTHELVDEVCDEERFTKVVTAGLNQIRNGVHDGLFTANFPGEENWAIAHRVLVPAFGPLSIRGMFDEMYDIATQLVMKWARHGPTVPIMVTDDFTRLTLDTIALCAMGTRFNSFYHEEMHPFVEAMVGLLQGSGDRARRPALLNNLPTSENSKYWDDIAFLRNLAQELVEARRKNPEDKKDLLNALILGRDPKTGKGLTDESIIDNMITFLIAGHETTSGLLSFLFYYLLKTPNAYKKAQEEVDSVVGRRKITVEDMSRLPYLNAVMRETLRLRSTAPLIAVHAHPEKNKEDPVTLGGGKYVLNKDEPIVIILDKLHRDPQVYGPDAEEFKPERMLDENFEKLPKNAWKPFGNGMRACIGRPFAWQEALLVVAILLQNFNFQMDDPSYNLHIKQTLTIKPKDFHMRATLRHGLDATKLGIALSGSADRAPPESSGAASRVRKQATPPAGQLKPMHIFFGSNTGTCETFARRLADDAVGYGFAADVQSLDSAMQNVPKDEPVVFITASYEGQPPDNAAHFFEWLSALKENELEGVNYAVFGCGHHDWQATFHRIPKAVNQLVAEHGGNRLCDLGLADAANSDMFTDFDSWGESTFWPAITSKFGGGKSDEPKPSSSLQVEVSTGMRASTLGLQLQEGLVIDNQLLSAPDVPAKRMIRFKLPSDMSYRCGDYLAVLPVNPTSVVRRAIRRFDLPWDAMLTIRKPSQAPKGSTSIPLDTPISAFELLSTYVELSQPASKRDLTALADAAITDADAQAELRYLASSPTRFTEEIVKKRMSPLDLLIRYPSIKLPVGDFLAMLPPMRVRQYSISSSPLADPSECSITFSVLNAPALAAASLPPAERAEAEQYMGVASTYLSELKPGERAHIAVRPSHSGFKPPMDLKAPMIMACAGSGLAPFRGFIMDRAEKIRGRRSSVGADGQLPEVEQPAKAILYVGCRTKGKDDIHATELAEWAQLGAVDVRWAYSRPEDGSKGRHVQDLMLEDREELVSLFDQGARIYVCGSTGVGNGVRQACKDIYLERRRQLRQAARERGEEVPAEEDEDAAAEQFLDNLRTKERYATDVFT

SEQ ID NO: 33 - protein, 1083 aa, A. nidulans
MAEIPEPKGLPLIGNIGTIDQEFPLGSMVALAEEHGEIYRLRFPGRTVVVVSTHALVNETCDEKRFRKSVNSALAHVREGVHDGLFTAKMGEVNWEIAHRVLMPAFGPLSIRGMFDEMHDIASQLALKWARYGPDCPIMVTDDFTRLTLDTLALCSMGYRFNSYYSPVLHPFIEAMGDFLTEAGEKPRRPPLPAVFFRNRDQKFQDDIAVLRDTAQGVLQARKEGKSDRNDLLSAMLRGVDSQTGQKMTDESIMDNLITFLIAGHETTSGLLSFVFYQLLKHPETYRTAQQEVDNVVGQGVIEVSHLSKLPYINSVLRETLRLNATIPLFTVEAFEDTLLAGKYPVKAGETIVNLLAKSHLDPEVYGEDALEFKPERMSDELFNARLKQFPSAWKPFGNGMRACIGRPFAWQEALLVMAMLLQNFDFSLADPNYDLKFKQTLTIKPKDMFMKARLRHGLTPTTLERRLAGLAVESATQDKIVTNPADNSVTGTRLTILYGSNSGTCETLARRIAADAPSKGFHVMRFDGLDSGRSALPTDHPVVIVTSSYEGQPPENAKQFVSWLEELEQQNESLQLKGVDFAVFGCFKEWAQTFHRIPKLVDSLLEKLGGSRLTDLGLADVSTDELFSTFETWADDVLWPRLVAQYGADGKTQAHGSSAGHEAASNAAVEVTVSNSRTQALRQDVGQAMVVETRLLTAESEKERRKKHLEIRLPDGVSYTAGDYLAVLPINPPETVRRAMRQFKLSWDAQITIAPSGPTTALPTDGPIAANDIFSTYVELSQPATRKDLRIMADATTDPDVQKILRTYANETYTAEILTKSISVLDILEQHPAIDLPLGTFLLMLPSMRMRQYSISSSPLLTPTTATITISVLDAPSRSRSNGSRHLGVATSYLDSLSVGDHLQVTVRKNPSSGFRLPSEPETTPMICIAAGSGIAPFRAFLQERAVMMEQDKDRKLAPALLFFGCRAPGIDDLYREQLEEWQARGVVDARWAFSRQSDDTKGCRHVDDRILADREDVVKLWRDGARVYVCGSGALAQSVRSAMVTVLRDEMETTGDGSDNGKAEKWFDEQRNVRYVMDVFD

SEQ ID NO: 34 - protein, 1054 aa, A. oryzae
MRQNDNEKQICPIPGPQGLPFLGNILDIDLDNGTMSTLKIAKTYYPIFKFTFAGETSIVINSVALLSELCDETRFHKHVSFGLELLRSGTHDGLFTAYDHEKNWELAHRLLVPAFGPLRIREMFPQMHDIAQQLCLKWQRYGPRRPLNLVDDFTRTTLDTIALCAMGYRFNSFYSEGDFHPFIKSMVRFLKEAETQATLPSFISNLRVRAKRRTQLDIDLMRTVCREIVTERRQTNLDHKNDLLDTMLTSRDSLSGDALSDESIIDNILTFLVAGHETTSGLLSFAVYYLLTTPDAMAKAAHEVDDVVGDQELTIEHLSMLKYLNAILRETLRLMPTAPGFSVTPYKPEIIGGKYEVKPGDSLDVFLAAVHRDPAVYGSDADEFRPERMSDEHFQKLPANSWKPFGNGKRSCIGRAFAWQEALMILALILQSFSLNLVDRGYTLKLKESLTIKPDNLWAYATPRPGRNVLHTRLALQTNSTHPEGLMSLKHETVESQPATILYGSNSGTCEALAHRLAIEMSSKGRFVCKVQPMDAIEHRRLPRGQPVIIITGSYDGRPPENARHFVKWLQSLKGNDLEGIQYAVFGCGLPGHHDWSTTFYKIPTLIDTIMAEHGGARLAPRGSADTAEDDPFAELESWSERSVWPGLEAAFDLVRHNSSDGTGKSTRITIRSPYTLRAAHETAVVHQVRVLTSAETTKKVHVELALPDTINYRPGDHLAILPLNSRQSVQRVLSLFQIGSDTILYMTSSSATSLPTDTPISAHDLLSGYVELNQVATPTSLRSLAAKATDEKTAEYLEALATDRYTTEVRGNHLSLLDILESYSVPSIEIQHYIQMLPLLRPRQYTISSSPRLNRGQASLTVSVMERADVGGPRNCAGVASNYLASCTPGSILRVSLRQANPDFRLPDESCSHPIIMVAAGSGIAPFRAFVQERSVRQKEGIILPPAFLFFGCRRADLDDLYREELDAFEEQGVVTLFRAFSRAQSESHGCKYVQDLLWMERVRVKTLWGQDAKVFVCGSVRMNEGVKAIISKIVSPTPTEELARRYIAETFI

SEQ ID NO: 35 - protein, 1103 aa, A. oryzae
MSTPKAEPVPIPGPRGVPLMGNILDIESEIPLRSLEMMADTYGPIYRLTTFGFSRCMISSHELAAEVFDEERFTKKIMAGLSELRHGIHDGLFTAHMGEENWEIAHRVLMPAFGPLNIQNMFDEMHDIATQLVMKWARQGPKQKIMVTDDFTRLTLDTIALCAMGTRFNSFYSEEMHPFVDAMVGMLKTAGDRSRRPGLVNNLPTTENNKYWEDIDYLRNLCKELVDTRKKNPTDKKDLLNALINGRDPKTGKGMSYDSIIDNMITFLIAGHETTSGSLSFAFYNMLKNPQAYQKAQEEVDRVIGRRRITVEDLQKLPYITAVMRETLRLTPTAPAIAVGPHPTKNHEDPVTLGNGKYVLGKDEPCALLLGKIQRDPKVYGPDAEEFKPERMLDEHFNKLPKHAWKPFGNGMRACIGRPFAWQEALLVIAMLLQNFNFQMDDPSYNIQLKQTLTIKPNHFYMRAALREGLDAVHLGSALSASSSEHADHAAGHGKAGAAKKGADLKPMHVYYGSNTGTCEAFARRLADDATSYGYSAEVESLDSAKDSIPKNGPVVFITASYEGQPPDNAAHFFEWLSALKGDKPLDGVNYAVFGCGHHDWQTTFYRIPKEVNRLVGENGANRLCEIGLADTANADIVTDFDTWGETSFWPAVAAKFGSNTQGSQKSSTFRVEVSSGHRATTLGLQLQEGLVVENTLLTQAGVPAKRTIRFKLPTDTQYKCGDYLAILPVNPSTVVRKVMSRFDLPWDAVLRIEKASPSSSKHISIPMDTQVSAYDLFATYVELSQPASKRDLAVLADAAAVDPETQAELQAIASDPARFAEISQKRISVLDLLLQYPSINLAIGDFVAMLPPMRVRQYSISSSPLVDPTECSITFSVLKAPSLAALTKEDEYLGVASTYLSELRSGERVQLSVRPSHTGFKPPTELSTPMIMACAGSGLAPFRGFVMDRAEKIRGRRSSGSMPEQPAKAILYAGCRTQGKDDIHADELAEWEKIGAVEVRRAYSRPSDGSKGTHVQDLMMEDKKELIDLFESGARIYVCGTPGVGNAVRDSIKSMFLERREEIRRIAKEKGEPVSDDDEETAFEKFLDDMKTKERYTTDIFA

SEQ ID NO: 36 - protein, 1066 aa, F. oxysporum
MAESVPIPEPPGYPLIGNLGEFTSNPLSDLNRLADTYGPIFRLRLGAKAPIFVSSNSLINEVCDEKRFKKTLKSVLSQVREGVHDGLFTAFEDEPNWGKAHRILVPAFGPLSIRGMFPEMHDIATQLCMKFARHGPRTPIDTSDNFTRLALDTLALCAMDFRFYSYYKEELHPFIEAMGDFLTESGNRNRRPPFAPNFLYRAANEKFYGDIALMKSVADEVVAARKASPSDRKDLLAAMLNGVDPQTGEKLSDENITNQLITFLIAGHETTSGTLSFAMYQLLKNPEAYSKVQKEVDEVVGRGPVLVEHLTKLPYISAVLRETLRLNSPITAFGLEAIDDTFLGGKYLVKKGEIVTALLSRGHVDPVVYGNDADKFIPERMLDDEFARLNKEYPNCWKPFGNGKRACIGRPFAWQESLLAMVVLFQNFNFTMTDPNYALEIKQTLTIKPDHFYINATLRHGMTPTELEHVLAGNGATSSSTHNIKAAANLDAKAGSGKPMAIFYGSNSGTCEALANRLASDAPSHGFSATTVGPLDQAKQNLPEDRPVVIVTASYEGQPPSNAAHFIKWMEDLDGNDMEKVSYAVFACGHHDWVETFHRIPKLVDSTLEKRGGTRLVPMGSADAATSDMFSDFEAWEDIVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKDTIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPLRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 37 - protein, 1115 aa, G. moniliformis
MSATALFTRRSVSTSNPELRPIPGPKPLPLLGNLFDFDFDNLTKSLGELGKIHGPIYSITFGASTEIMVTSREIAQELCDETRFCKLPGGALDVMKAVVGDGLFTAETSNPKWAIAHRIITPLFGAMRIRGMFDDMKDICEQMCLRWARFGPDEPLNVCDNMTKLTLDTIALCTIDYRFNSFYRENGAAHPFAEAVVDVMTESFDQSNLPDFVNNYVRFRAMAKFKRQAAELRRQTEELIAARRQNPVDRDDLLNAMLSAKDPKTGEGLSPESIVDNLLTFLIAGHETTSSLLSFCFYYLLENPHVLRRVQQEVDTVVGSDTITVDHLSSMPYLEAVLRETLRLRDPGPGFYVKPLKDEVVAGKYAVNKDQPLFIVFDSVHRDQSTYGADADEFRPERMLKDGFDKLPPCAWKPFGNGVRACVGRPFAMQQAILAVAMVLHKFDLVKDESYTLKYHVTMTVRPVGFTMKVRLRQGQRATDLAMGLHRGHSQEASAAASPSRASLKRLSSDVNGDDTDHKSQIAVLYASNSGSCEALAYRLAAEATERGFGIRAVDVVNNAIDRIPVGSPVILITASYNGEPADDAQEFVPWLKSLESGRLNGVKFAVFGNGHRDWANTLFAVPRLIDSELARCGAERVSLMGVSDTCDSSDPFSDFERWIDEKLFPELETPHGPGGVKNGDRAVPRQELQVSLGQPPRITMRKGYVRAIVTEARSLSSPGVPEKRHLELLLPKDFNYKAGDHVYILPRNSPRDVVRALSYFGLGEDTLITIRNTARKLSLGLPLDTPITATDLLGAYVELGRTASLKNLWTLVDAAGHGSRAALLSLTEPERFRAEVQDRHVSILDLLERFPDIDLSLSCFLPMLAQIRPRAYSFSSAPDWKPGHATLTYTVVDFATPATQGINGSSKSKAVGDGTAVVQRQGLASSYLSSLGPGTSLYVSLHRASPYFCLQKSTSLPVIMVGAGTGLAPFRAFLQERRMAAEGAKQRFGPALLFFGCRGPRLDSLYSVELEAYETIGLVQVRRAYSRDPSAQDAQGCKYVTDRLGKCRDEVARLWMDGAQVLVCGGKKMANDVLEVLGPMLLEIDQKRGETTAKTVVEWRARLDKSRYVEEVYV

SEQ ID NO: 38 - protein, 1069 aa, G. zeae
MAESVPIPEPPGYPLIGNLGEFKTNPLNDLNRLADTYGPIFRLHLGSKTPTFVSSNAFINEVCDEKRFKKTLKSVLSVVREGVHDGLFTAFEDEPNWGKAHRILIPAFGPLSIRNMFPEMHEIANQLCMKLARHGPHTPVDASDNFTRLALDTLALCAMDFRFNSYYKEELHPFIEAMGDFLLESGNRNRRPAFAPNFLYRAANDKFYADIALMKSVADEVVATRKQNPTDRKDLLAAMLEGVDPQTGEKLSDDNITNQLITFLIAGHETTSGTLSFAMYHLLKNPEAYNKLQKEIDEVIGRDPVTVEHLTKLPYLSAVLRETLRISSPITGFGVEAIEDTFLGGKYLIKKGETVLSVLSRGHVDPVVYGPDAEKFVPERMLDDEFARLNKEFPNCWKPFGNGKRACIGRPFAWQESLLAMALLFQNFNFTQTDPNYELQIKQNLTIKPDNFFFNCTLRHGMTPTDLEGQLAGKGATTSIASHIKAPAASKGAKASNGKPMAIYYGSNSGTCEALANRLASDAAGHGFSASVIGTLDQAKQNLPEDRPVVIVTASYEGQPPSNAAHFIKWMEDLAGNEMEKVSYAVFGCGHHDWVDTFLRIPKLVDTTLEQRGGTRLVPMGSADAATSDMFSDFEAWEDTVLWPSLKEKYNVTDDEASGQRGLLVEVTTPRKTTLRQDVEEALVVSEKTLTKTGPAKKHIEIQLPSGMTYKAGDYLAILPLNPRKTVSRVFRRFSLAWDSFLKIQSDGPTTLPINIAISAFDVFSAYVELSQPATKRNILALSEATEDKATIQELEKLAGDAYQEDVSAKKVSVLDLLEKYPAVALPISSYLAMLPPMRVRQYSISSSPFADPSKLTLTYSLLDAPSLSGQGRHVGVATNFLSQLIAGDKLHISVRASSAAFHLPSDPETTPIICVAAGTGLAPFRGFIQERAAMLAAGRKLAPALLFFGCRDPENDDLYAEELARWEQMGAVDVRRAYSRATDKSEGCKYVQDRIYHDRADVFKVWDQGAKVFICGSREIGKAVEDICVRLAMERSEATQEGKGATEEKAREWFERSRNERFATDVFD

SEQ ID NO: 39 - protein, 1066 aa, G. zeae
MAIKDGGKKSGQIPGPKGLPVLGNLFDLDLSDSLTSLINIGQKYAPIFSLELGGHREVMICSRDLLDELCDETRFHKIVTGGVDKLRPLAGDGLFTAQHGNHDWGIAHRILMPLFGPLKIREMFDDMQDVSEQLCLKWARLGPSATIDVANDFTRLTLDTIALCTMGYRFNSFYSNDKMHPFVDSMVAALIDADKQSMFPDFIGACRVKALSAFRKHAAIMKGTCNELIQERRKNPIEGTDLLTAMMEGKDPKTGEGMSDDLIVQNLITFLIAGHETTSGLLSFAFYYLLENPHTLEKARAEVDEVVGDQALNVDHLTKMPYVNMILRETLRLMPTAPGFFVTPHKDEIIGGKYAVPANESLFCFLHLIHRDPKVWGADAEEFRPERMADEFFEALPKNAWKPFGNGMRGCIGREFAWQEAKLITVMILQNFELSKADPSYKLKIKQSLTIKPDGFNMHAKLRNDRKVSGLFKAPSLSSQQPSLSSRQSINAINAKDLKPISIFYGSNTGTCEALAQKLSADCVASGFMPSKPLPLDMATKNLSKDGPNILLAASYDGRPSDNAEEFTKWAESLKPGELEGVQFAVFGCGHKDWVSTYFKIPKILDKCLADAGAERLVEIGLTDASTGRLYSDFDDWENQKLFTELSKRQGVTPTDDSHLELNVTVIQPQNNDMGGNFKRAEVVENTLLTYPGVSRKHSLLLKLPKDMEYTPGDHVLVLPKNPPQLVEQAMSCFGVDSDTALTISSKRPTFLPTDTPILISSLLSSLVELSQTVSRTSLKRLADFADDDDTKACVERIAGDDYTVEVEEQRMSLLDILRKYPGINMPLSTFLSMLPQMRPRTYSFASAPEWKQGHGMLLFSVVEAEEGTVSRPGGLATNYMAQLRQGDSILVEPRPCRPELRTTMMLPEPKVPIIMIAVGAGLAPFLGYLQKRFLQAQSQRTALPPCTLLFGCRGAKMDDICRAQLDEYSRAGVVSVHRAYSRDPDSQCKYVQGLVTKHSETLAKQWAQGAIVMVCSGKKVSDGVMNVLSPILFAEEKRSGMTGADSVDVWRQNVPKERMILEVFG

SEQ ID NO: 40 - protein, 1130 aa, M. grisea
MFFLSSSLAYMAATQSRDWASFGVSLPSTALGRHLQAAMPFLSEENHKSQGTVLIPDAQGPIPFLGSVPLVDPELPSQSLQRLARQYGEIYRFVIPGRQSPILVSTHALVNELCDEKRFKKKVAAALLGLREAIHDGLFTAHNDEPNWGIAHRILMPAFGPMAIKGMFDEMHDVASQMILKWARHGSTTPIMVSDDFTRLTLDTIALCSMGYRFNSFYHDSMHEFIEAMTCWMKESGNKTRRLLPDVFYRTTDKKWHDDAEILRRTADEVLKARKENPSGRKDLLTAMIEGVDPKTGGKLSDSSIIDNLITFLIAGHETTSGMLSFAFYLLLKNPTAYRKAQQEIDDLCGREPITVEHLSKMPYITAVLRETLRLYSTIPAFVVEAIEDTVVGGKYAIPKNHPIFLMIAESHRDPKVYGDDAQEFEPERMLDGQFERRNREFPNSWKPFGNGMRGCIGRAFAWQEALLITAMLLQNFNFVMHDPAYQLSIKENLTLKPDNFYMRAILRHGMSPTELERSISGVAPTGNKTPPRNATRTSSPDPEDGGIPMSIYYGSNSGTCESLAHKLAVDASAQGFKAETVDVLDAANQKLPAGNRGPVVLITASYEGLPPDNAKHFVEWLENLKGGDELVDTSYAVFGCGHQDWTKTFHRIPKLVDEKLAEHGAVRLAPLGLSNAAHGDMFVDFETWEFETLWPALADRYKTGAGRQDAAATDLTAALSQLSVEVSHPRAADLRQDVGEAVVVAARDLTAPGAPPKRHMEIRLPKTGGRVHYSAGDYLAVLPVNPKSTVERAMRRFGLAWDAHVTIRSGGRTTLPTGAPVSAREVLSSYVELTQPATKRGIAVLAGAVTGGPAAEQEQAKAALLDLAGDSYALEVSAKRVGVLDLLERFPACAVPFGTFLALLPPMRVRQYSISSSPLWNDEHATLTYSVLSAPSLADPARTHVGVASSYLAGLGEGDHLHVALRPSHVAFRLPSPETPVVCVCAGSGMAPFRAFAQERAALVGAGRKVAPLLLFFGCREPGVDDLYREELEGWEAKGVLSVRRAYSRRTEQSEGCRYVQDRLLKNRAEVKSLWSQDAKVFVCGSREVAEGVKEAMFKVVAGKEGSSEEVQAWYEEVRNVRYASDIFD

SEQ ID NO: 41 - protein, 1108 aa, N. crassa
MSSDETPQTIPIPGPPGLPLVGNSFDIDTEFPLGSMLNFADQYGEIFRLNFPGRNTVFVTSQALVHELCDEKRFQKTVNSALHEIRHGIHDGLFTARNDEPNWGIAHRILMPAFGPMAIQNMFPEMHEIASQLALKWARHGPNQSIKVTDDFTRLTLDTIALCSMDYRFNSYYHDDMHPFIDAMASFLVESGNRSRRPALPAFMYSKVDRKFYDDIRVLRETAEGVLKSRKEHPSERKDLLTAMLDGVDPKTGGKLSDDSIIDNLITFLIAGHETTSGLLSFAFVQLLKNPETYRKAQKEVDDVCGKGPIKLEHMNKLHYIAAVLRETLRLCPTIPVIGVESKEDTVIGGKYEVSKGQPFALLFAKSHVDPAVYGDTANDFDPERMLDENFERLNKEFPDCWKPFGNGMRACIGRPFAWQEALLVMAVCLQNFNFMPEDPNYTLQYKQTLTTKPKGFYMRAMLRDGMSALDLERRLKGELVAPKPTAQGPVSGQPKKSGEGKPISIYYGSNTGTCETFAQRLASDAEAHGFTATIIDSLDAANQNLPKDRPVVFITASYEGQPPDNAALFVGWLESLTGNELEGVQYAVFGCGHHDWAQTFHRIPKLVDNTVSERGGDRICSLGLADAGKGEMFTEFEQWEDEVFWPAMEEKYEVSRKEDDNEALLQSGLTVNFSKPRSSTLRQDVQEAVVVDAKTITAPGAPPKRHIEVQLSSDSGAYRSGDYLAVLPINPKETVNRVMRRFQLAWDTNITIEASRQTTILPTGVPMPVHDVLGAYVELSQPATKKNILALAEAADNAETKATLRQLAGPEYTEKITSRRVSILDLLEQFPSIPLPFSSFLSLLPPMRVRQYSISSSPLWNPSHVTLTYSLLESPSLSNPDKKHVGVATSYLASLEAGDKLNVSIRPSHKAFHLPVDADKTPLIMIAAGSGLAPFRGFVQERAAQIAAGRSLAPAMLFYGCRHPEQDDLYRDEFDKWESIGAVSVRRAFSRCPESQETKGCKYVGDRLWEDREEVTGLWDRGAKVYVCGSREVGESVKKVVVRIALERQKMIVEAREKGELDSLPEGIVEGLKLKGLTVEDVEVSEERALKWFEGIRNERYATDVFD

SEQ ID NO: 42 - protein, 561 aa, Oryza sativa
MAAAAAAAVPCVPFLCPPPPPLVSPRLRRGHVRLRLRPPRSSGGGGGGGAGGDEPPITTSWVSPDWLTALSRSVATRLGGGDDSGIPVASAKLDDVRDLLGGALFLPLFKWFREEGPVYRLAAGPRDLVVVSDPAVARHVLRGYGSRYEKGLVAEVSEFLFGSGFAIAEGALWTVRRRSVVPSLHKRFLSVMVDRVFCKCAERLVEKLETSALSGKPVNMEARFSQMTLDVIGLSLFNYNFDSLTSDSPVIDAVYTALKEAELRSTDLLPYWKIDLLCKIVPRQIKAEKAVNIIRNTVEDLITKCKKIVDAENEQIEGEEYVNEADPSILRFLLASREEVTSVQLRDDLLSMLVAGHETTGSVLTWTIYLLSKDPAALRRAQAEVDRVLQGRLPRYEDLKELKYLMRCINESMRLYPHPPVLIRRAIVDDVLPGNYKIKAGQDIMISVYNIHRSPEVWDRADDFIPERFDLEGPVPNETNTEYRFIPFSGGPRKCVGDQFALLEAIVALAVVLQKMDIELVPDQKINMTTGATIHTTNGLYMNVSLRKVDREPDFALSGSR

SEQ ID NO: 43 - protein, 467 aa, Artificial Sequence
  Sythetic Chimeric heme enzyme C2G9
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDRMIAERKANPDENIKDLLSLMLYAKDPVTGETLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPISKGQPVTVLIPKLHRDQNAWGPDAEDFRPERFEDPSSIPHHAYKPFGNGQRACIGMQFALHEATLVLGMILKYFTLIDHENYELDIKQTLTLKPGDFHISVQSRHQEAIHADVQAAE

SEQ ID NO: 44 - protein, 467 aa, Artificial Sequence
  Sythetic Chimeric heme enzyme X7
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 45 - protein, 466 aa, Artificial Sequence
  Sythetic Chimeric heme enzyme X7-12
MTIKEMPQPKTFGELKNLPLLNTDKPVQALMKIADELGEIFKFEAPGRVTRYLSSQRLIKEACDEERFDKSIEGALEKVRAFSGDGLATSWTHEPNWRKAHNILMPTFSQRAMKDYHEKMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 46 - protein, 465 aa, Artificial Sequence
  Sythetic Chimeric heme enzyme C2E6
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPST

SEQ ID NO: 47 - protein, 467 aa, Artificial Sequence
  Sythetic Chimeric heme enzyme X7-9
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 48 - protein, 467 aa, Artificial Sequence
  Sythetic Chimeric heme enzyme C2B12
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFATYFLLKHPDKLKKAYEEVDRVLTDAAPTYKQVLELTYIRMILNESLRLWPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 49 - protein, 467 aa, Artificial Sequence
  Sythetic Chimeric heme enzyme TSP234
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 50 - protein, 463 aa, Artificial Sequence
  Synthetic WT-AxA (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAAIGQQFALHEATLVLGMMLKHFDFEDHTNYELDIKETLSLKPKGFVVKAKSKKIPLGGIPSPST

SEQ ID NO: 51 - protein, 463 aa, Artificial Sequence
  Synthetic WT-AxD (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRADIGQQFALHEATLVLGMMLKHFDFEDHTNYELDIKETLSLKPKGFVVKAKSKKIPLGGIPSPST

SEQ ID NO: 52 - protein, 463 aa, Artificial Sequence
  Synthetic WT-AxH (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAHIGQQFALHEATLVLGMMLKHFDFEDHTNYELDIKETLSLKPKGFVVKAKSKKIPLGGIPSPST

SEQ ID NO: 53 - protein, 463 aa, Artificial Sequence
  Synthetic WT-AxK (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAKIGQQFALHEATLVLGMMLKHFDFEDHTNYELDIKETLSLKPKGFVVKAKSKKIPLGGIPSPST

SEQ ID NO: 54 - protein, 463 aa, Artificial Sequence
  Synthetic WT-AxM (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAMIGQQFALHEATLVLGMMLKHFDFEDHTNYELDIKETLSLKPKGFVVKAKSKKIPLGGIPSPST

SEQ ID NO: 55 - protein, 463 aa, Artificial Sequence
  Synthetic WT-AxN (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRANIGQQFALHEATLVLGMMLKHFDFEDHTNYELDIKETLSLKPKGFVVKAKSKKIPLGGIPSPST

SEQ ID NO: 56 - protein, 1047 aa, Artificial Sequence
  Synthetic P450 BM3 enzyme variant BM3-CIS-T438S-AxA
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAAIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 57 - protein, 1047 aa, Artificial Sequence
  Synthetic P450 BM3 enzyme variant BM3-CIS-T438S-AxD
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRADIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 58 - protein, 1047 aa, Artificial Sequence
  Synthetic P450 BM3 enzyme variant BM3-CIS-T438S-AxM
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAMIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 59 - protein, 1047 aa, Artificial Sequence
  Synthetic P450 BM3 enzyme variant BM3-CIS-T438S-AxY
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAYIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 60 - protein, 1047 aa, Artificial Sequence
  Synthetic P450 BM3 enzyme variant BM3-CIS-T438S-AxT
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRATIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 61 - protein, 133 aa, M. infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLVGDCLLKSIQEYLGQGFTEEAKAAWTKVYGIAAQVMTAE

SEQ ID NO: 62 - protein, 132 aa, Bacillus subtilis
MGQSFNAPYEAIGEELLSQLVDTFYERVASHPLLKPIFPSDLTETARKQKQFLTQYLGGPPLYTEEHGHPMLRARHLPFPITNERADAWLSCMKDAMDHVGLEGEIREFLFGRLELTARHMVNQTEAEDRSS

SEQ ID NO: 63 - protein, 368 aa, Sulfolobus acidocaldarius
MYDWFSEMRKKDPVYYDGNIWQVFSYRYTKEVLNNFSKFSSDLTGYHERLEDLRNGKIRFDIPTRYTMLTSDPPLHDELRSMSADIFSPQKLQTLETFIRETTRSLLDSIDPREDDIVKKLAVPLPIIVISKILGLPIEDKEKFKEWSDLVAFRLGKPGEIFELGKKYLELIGYVKDHLNSGTEVVSRVVNSNLSDIEKLGYIILLLIAGNETTTNLISNSVIDFTRFNLWQRIREENLYLKAIEEALRYSPPVMRTVRKTKERVKLGDQTIEEGEYVRVWIASANRDEEVFHDGEKFIPDRNPNPHLSFGSGIHLCLGAPLARLEARIAIEEFSKRFRHIEILDTEKVPNEVLNGYKRLVVRLKSNE

SEQ ID NO: 64 - protein, 133 aa, Artificial Sequence
  Synthetic variant of  M. infernorum
  VARIANT (28)..(29): Xaa = any amino acid
  VARIANT (32)..(32): Xaa = any amino acid
  VARIANT (54)..(54): Xaa = any amino acid
  VARIANT (95)..(95): Xaa = any amino acid
MIDQKEKELIKESWKRIEPNKNEIGLLXXANXFKEEPTVSVLFQNPISSQSRKXMQVLGILVQGIDNLEGLIPTLQDLGRRHKQYGVVDSHYPLXGDCLLKSIQEYLGQGFTEEAKAAWTKVYGIAAQVMTAE

SEQ ID NO: 65 - protein, 133 aa, Artificial Sequence; identical to SEQ ID NO: 64
  Synthetic variant of M. infernorum
  VARIANT (28)..(28): Xaa = Phe or Ser
  VARIANT (29)..(29): Xaa = Tyr or Ala
  VARIANT (32)..(32): Xaa = Leu, Ala, Cys or Thr
  VARIANT (54)..(54): Xaa = Leu or Ser
  VARIANT (95)..(95): Xaa = Val or Phe

SEQ ID NO: 66 - protein, 133 aa, Artificial Sequence
  Synthetic variant of M. infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLFGDCLLKSIQEYLGQGFTEEAKAAWTKVYGIAAQVMTAE

SEQ ID NO: 67 - protein, 132 aa, Artificial Sequence
  Synthetic variant of the B. subtilis
  VARIANT (45)..(45): Xaa = any amino acid
  VARIANT (49)..(49): Xaa = any amino acid
MGQSFNAPYEAIGEELLSQLVDTFYERVASHPLLKPIFPSDLTEXARKXKQFLTQYLGGPPLYTEEHGHPMLRARHLPFPITNERADAWLSCMKDAMDHVGLEGEIREFLFGRLELTARHMVNQTEAEDRSS

SEQ ID NO: 68 - protein, 132 aa, Artificial Sequence; identical to SEQ ID NO: 67
  Synthetic variant of B. subtilis
  VARIANT (45)..(45): Xaa = Thr, Leu, Phe or Ala
  VARIANT (49)..(49): Xaa = Gln, Leu, Phe or Ala

SEQ ID NO: 69 - protein, 132 aa, Artificial Sequence; identical to SEQ ID NO: 67
  Synthetic variant of B. subtilis
  VARIANT (45)..(45): Xaa = Leu, Phe, or Ala
  VARIANT (49)..(49): Xaa = Leu, Phe, or Ala

SEQ ID NO: 70 - protein, 368 aa, Artificial Sequence
  Synthetic variant of CYP119 from Sulfolobus acidocaldarius
  VARIANT (315)..(315): Xaa = any amino acid
MYDWFSEMRKKDPVYYDGNIWQVFSYRYTKEVLNNFSKFSSDLTGYHERLEDLRNGKIRFDIPTRYTMLTSDPPLHDELRSMSADIFSPQKLQTLETFIRETTRSLLDSIDPREDDIVKKLAVPLPIIVISKILGLPIEDKEKFKEWSDLVAFRLGKPGEIFELGKKYLELIGYVKDHLNSGTEVVSRVVNSNLSDIEKLGYIILLLIAGNETTTNLISNSVIDFTRFNLWQRIREENLYLKAIEEALRYSPPVMRTVRKTKERVKLGDQTIEEGEYVRVWIASANRDEEVFHDGEKFIPDRNPNPHLSFGSGIXLCLGAPLARLEARIAIEEFSKRFRHIEILDTEKVPNEVLNGYKRLVVRLKSNE

(19) United States
(12) Patent Application Publication
(10) Pub. No.: US 2018/0148745 A1
(43) Pub. Date: May 31, 2018

(54) HEMOPROTEIN CATALYSTS FOR IMPROVED ENANTIOSELECTIVE ENZYMATIC SYNTHESIS OF TICAGRELOR

(71) Applicants: California Institute of Technology, Pasadena, CA (US); Provivi, Inc., Santa Monica, CA (US)

(72) Inventors: Francis H. Arnold, La Canada, CA (US); Hans Renata, Pasadena, CA (US); John A. McIntosh, Pasadena, CA (US); Russell D. Lewis, Pasadena, CA (US); Sek Bik Jennifer Kan, Pasadena, CA (US); Kari Hernandez, Pasadena, CA (US); Pedro Coelho, Santa Monica, CA (US); David Rozzell, Santa Monica, CA (US); Chen Zhang, Santa Monica, CA (US)

(73) Assignees: California Institute of Technology, Pasadena, CA (US); Provivi, Inc., Santa Monica, CA (US)

(21) Appl. No.: 15/576,243

(22) PCT Filed: May 26, 2016
(86) PCT No.: PCT/US2016/034461

§ 371 Date: Nov. 21, 2017

Related U.S. Application Data
(60) Provisional application No. 62/166,582, filed on May 26, 2015.
(60) Provisional application No. 62/172,736, filed on Jun. 8, 2015.
(60) Provisional application No. 62/181,651, filed on Jun. 18, 2015.
(60) Provisional application No. 62/294,201, filed on Feb. 11, 2016.

(51) Int. Cl. C12P 7/62; C12P 7/26

(52) U.S. Cl. CPC C12P 7/62; C12P 7/26; C12Y 114/14001; C12Y 114/15003; C12Y 106/02004

(57) ABSTRACT

The present invention provides methods by which trans-(1R,2S)-2-(3,4-difluorophenyl)-cyclopropylamine and related cyclopropane compounds are prepared using synthetic strategies that include a biocatalytic cyclopropanation step.

### CROSS-REFERENCES TO RELATED APPLICATIONS

The present application claims priority to U.S. Provisional Patent Appl. No. 62/166,582, filed May 26, 2015; U.S. Provisional Patent Appl. No. 62/172,736, filed Jun. 8, 2015; U.S. Provisional Patent Appl. No. 62/181,651, filed Jun. 18, 2015; U.S. Provisional Patent Appl. No. 62/294,201, filed Feb. 11, 2016; which applications are incorporated herein by reference in their entirety.

### STATEMENT AS TO RIGHTS TO INVENTIONS MADE UNDER FEDERALLY SPONSORED RESEARCH AND DEVELOPMENT

This invention was made with government support under Grant No. 1403077 awarded by the National Science Foundation and Grant No. 1549855 awarded by the National Science Foundation Small Business Technology Transfer program. The government has certain rights in the invention.

### BACKGROUND OF THE INVENTION

U.S. Pat. Nos. 6,251,910 and 6,525,060 disclose a variety of triazolo[4,5-d]pyrimidine derivatives, processes for their preparation, pharmaceutical compositions comprising the derivatives, and methods of use thereof. These compounds act as P_{2T} (P2Y_{ADP} or P2T_{AC}) receptor antagonists and they are indicated for use in therapy as inhibitors of platelet activation, aggregation, and degranulation, promoters of platelet disaggregation and antithrombotic agents. Among them, Ticagrelor, [1S-(1α,2α,3˜(1S*,2R*),5β)]-3-[7-[2-(3,4-difluorophenyl)cyclopropyl]amino]-5-(propylthio)-3H-1,2,3-triazolo[4,5-d]pyrimidin-3-yl)-5-(2-hydroxyethoxy)-cyclopentane-1,2-diol, acts as an adenosine uptake inhibitor, a platelet aggregation inhibitor, a P2Y12 purinoceptor antagonist, and a coagulation inhibitor. It is indicated for the treatment of thrombosis, angina, ischemic heart diseases, and coronary artery diseases. Ticagrelor is represented by the following structural Formula I:

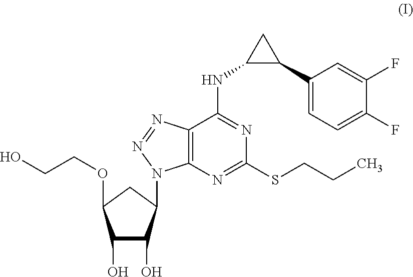

Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix) in the prevention of cardiovascular (CV) events including myocardial infarction (heart attacks), stroke, and cardiovascular death in patients with acute coronary syndrome (ACS). In 2014, the fourth year after its launch, ticagrelor reached worldwide sales of $485M and 25 tons in volume.

Various processes for the preparation of pharmaceutically active triazolo[4,5-d]pyrimidine cyclopentane compounds, preferably ticagrelor, their enantiomers, and their pharmaceutically acceptable salts are disclosed in U.S. Pat. Nos. 6,251,910; 6,525,060; 6,974,868; 7,067,663; 7,122,695 and 7,250,419; U.S. Patent Application Nos. 2007/0265282, 2008/0132719 and 2008/0214812; European Patent Nos. EP0996621 and EP1135391; and PCT Publication Nos. WO2008/018823 and WO2010/030224.

One of the useful intermediates in the synthesis of pharmaceutically active triazolo[4,5-d]pyrimidine cyclopentane compounds is the substituted phenylcyclopropylamine derivative of Formula XLIIa:

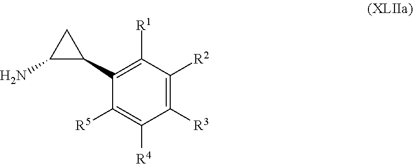

wherein R¹, R², R³, R⁴, and R⁵ are, each independently, selected from hydrogen and a halogen atom, wherein the halogen atom is F, Cl, Br or I; preferably, the halogen atom is F.

In the preparation of ticagrelor, trans-(1R,2S)-2-(3, 4-difluorophenyl)-cyclopropylamine of Formula IIa is a key intermediate:

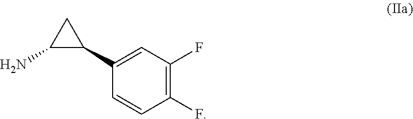 [SMILES: N[C@@H]1C[C@H]1c1ccc(F)c(F)c1]

According to U.S. Pat. No. 6,251,910 (hereinafter referred to as the '910 patent), the substituted phenylcyclopropylamine derivatives are prepared by a process as depicted in Scheme 1.

The process for the preparation of substituted phenylcyclopropylamine derivatives disclosed in the '910 patent involves the use of hazardous and explosive materials like sodium hydride, diazomethane and sodium azide. The process also involves the use of highly expensive chiral sultam auxiliary. Moreover, the yields of substituted phenylcyclopropylamine derivatives obtained are low to moderate, and the process involves column chromatographic purifications.

Methods involving column chromatographic purifications are generally undesirable for large-scale operations, thereby making the process commercially unfeasible. The use of explosive reagents like sodium hydride, diazomethane and sodium azide is not advisable, due to the handling difficulties, for scale up operations.

U.S. Pat. No. 7,122,695 (hereinafter referred to as the '695 patent) discloses a process for the preparation of substituted phenylcyclopropylamine derivatives, specifically trans-(1R,2S)-2-(3,4-difluorophenyl)cyclopropylamine and its mandelate salt. The synthesis is depicted in Scheme 2.

According to the '695 patent, the trans-(1R,2S)-2-(3,4-difluorophenyl)-cyclopropylamine is prepared by reacting 3,4-difluorobenzaldehyde with malonic acid in the presence of pyridine and piperidine to produce (E)-3-(3,4-difluorophenyl)-2-propenoic acid, followed by the reaction with thionyl chloride in the presence of pyridine in toluene to produce (E)-3-(3,4-difluorophenyl)-2-propenoyl chloride, which is then reacted with L-menthol in the presence of pyridine in toluene to produce (1R, 2S, 5R)-2-isopropyl 1-5-methylcyclohexyl (E)-3-(3,4-difluorophenyl)-2-propenoate. The (1R, 2S, 5R)-2-isopropyl-5-methylcyclohexyl (E)-3-(3,4-difluorophenyl)-2-propenoate is then reacted with dimethyl-sulfoxonium

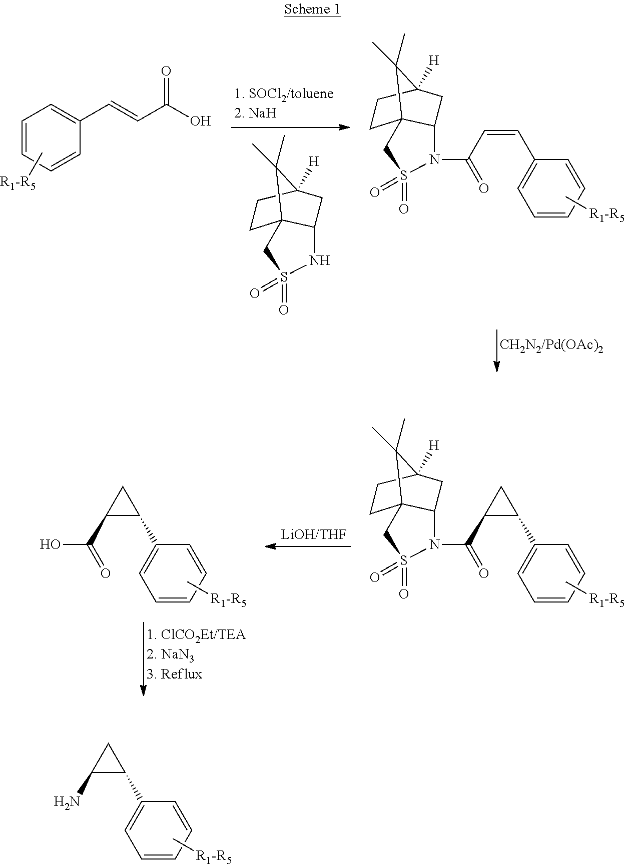

methylide in the presence of sodium hydroxide and sodium iodide in dimethylsulfoxide to produce a solution containing (1R,2S,5R)-2-isopropyl-5-methylcyclohexyl trans-2-(3,4-difluorophenyl)cyclopropanecarboxylate, followed by the diastereomeric separation to produce (1R,2S,5R)-2-isopropyl-5-methylcyclohexyl trans-(1R,2R)-2-(3,4-difluorophenyl)cyclopropanecarboxylate. The ester compound is hydrolyzed with sodium hydroxide in ethanol, followed by the acidification with hydrochloric acid to produce trans-(1R,2R)-2-(3,4-difluorophenyl)cyclopropanecarboxylic acid, followed by reaction with thionyl chloride in the presence of pyridine in toluene to produce trans-(1R, 2R)-2-(3,4-difluorophenyl)cyclopropanecarbonyl chloride, which is then reacted with sodium azide in the presence of tetrabutylammonium bromide and sodium carbonate in toluene to produce a reaction mass containing trans-(1R,2R)-2-(3,4-difluorophenyl) cyclopropanecarbonyl azide. The azide compound is then added to toluene while stirring at 100° C., followed by acid/base treatment to produce trans-(1R,2R)-2-(3,4-difluorophenyl)cyclopropylamine, which is then converted to its mandelate salt by reaction with R-(−)-mandelic acid in ethyl acetate.

The process disclosed in the '695 patent is lengthy thus resulting in a poor product yield. The process also involves the use of hazardous materials like pyridine and sodium azide.

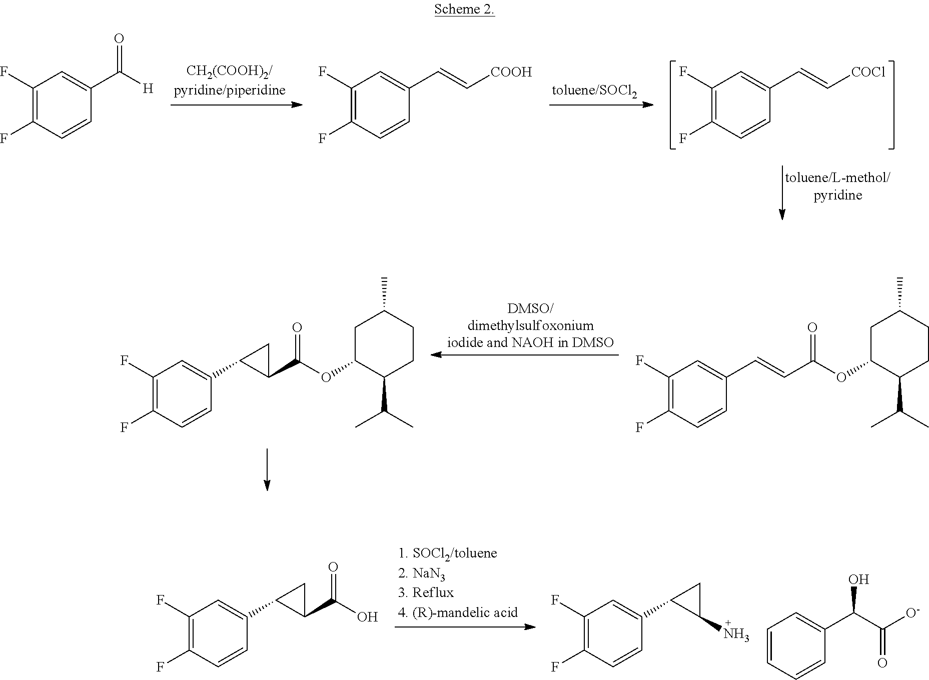

U.S. Patent Appl. Pub. No. 2008/0132719 (hereinafter referred to as the '719 publication) describes a process for the preparation of (1R,2S)-2-(3,4-difluorophenyl)-cyclopropane amine. The synthetic route is depicted in Scheme 3.

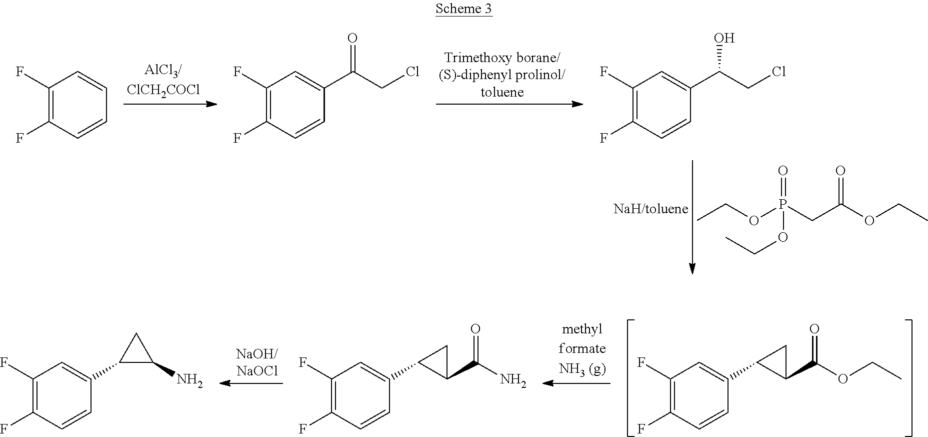

According to the '719 publication, the (1R, 2S)-2-(3, 4-difluorophenyl)-cyclopropane amine is prepared by reacting 1,2-difluorobenzene with chloroacetyl chloride in the presence of aluminum trichloride to produce 2-chloro-1-(3, 4-difluorophenyl)ethanone, followed by the reaction with trimethoxy borane and S-diphenylprolinol in toluene to produce 2-chloro-(1S)-(3,4-difluorophenyl)ethanol, which is then reacted with triethyl phosphonoacetate in the presence of sodium hydride in toluene to produce ethyl (1R, 2R)-trans-2-(3,4-difluorophenyl)cyclopropyl carboxylate. The ester compound is then reacted with methyl formate in the presence of OH ammonia to produce (1R, 2R)-trans-2-(3,4-difluorophenyl) cyclopropyl carboxamide, which is then reacted with sodium hydroxide and sodium hypochlorite to produce (1R, 2S)-2-(3, 4-difluorophenyl)-cyclopropane amine.

The process described in the '719 publication suffers from the disadvantages such as the use of explosive materials like sodium hydride and the use of expensive S-diphenylprolinol.

PCT Publication No. WO2008/018823 (hereinafter referred to as the '823 publication) describes a process for the preparation of (1R,2S)-2-(3,4-difluorophenyl)-1-cyclopropanamine. The synthetic route is depicted in Scheme 4.

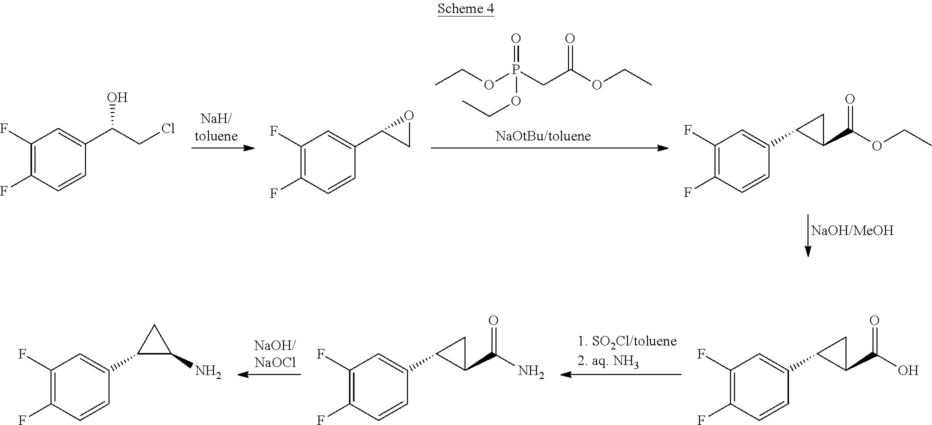

According to the '823 publication, the (1R, 2S)-2-(3,4-difluorophenyl)-1-cyclopropanamine is prepared by reacting (1S)-2-chloro-1-(3,4-difluorophenyl)-1-ethanol with sodium hydroxide in toluene to produce (2S)-2-(3,4-difluorophenyl)oxirane, followed by reaction with triethyl phosphonoacetate in the presence of sodium t-butoxide in toluene to produce ethyl (1R, 2R)-2-(3,4-difluorophenyl)-1-cyclopropanecarboxylate, which is then hydrolyzed with sodium hydroxide in methanol to produce (1R,2R)-2-(3,4-difluorophenyl)-1-cyclopropanecarboxylic acid. The resulting carboxylic acid compound is reacted with thionyl chloride in toluene to produce a solution of (1R, 2R)-2-(3,4-difluorophenyl)-1-cyclopropanecarbonyl chloride, followed by subsequent reaction with aqueous ammonia to produce (1R, 2R)-2-(3,4-difluorophenyl)-1-cyclopropanecarboxamide, which is then reacted with sodium hydroxide in the presence of sodium hypochlorite to produce (1R, 2S)-2-(3,4-difluorophenyl)-1-cyclopropanamine.

Bioorganic & Medicinal Chemistry, vol. 17(6), pages 2388-2399 (2009) discloses a process for the preparation of racemic trans-2-(3,4-difluorophenyl)cyclopropylamine and its acid addition salt. J. Med. Chem., vol. 20, No. 7, pages 934-939 (1977) discloses a process for the preparation of 1-aryl-3-nitro-1-propanones from 1-aryl-3-chloro-1-propanones. J. Org. Chem. 57, pages 3757-3759 (1992) discloses an intramolecular Mitsunobu displacement with carbon nucleophiles for preparation of nitrocyclopropanes from nitroalkanol.

These current methods for synthesizing trans-(1R,2S)-2-(3,4-difluorophenyl)-cyclopropylamine, a key intermediate in the synthesis of ticagrelor, are costly and laborious because they require numerous steps to achieve the cyclopropane intermediate. Based on the aforementioned drawbacks, improved methods for the preparation of substituted phenylcyclopropylamine derivatives at lab scale and in commercial scale operations is desired.

A need remains for improved methods of preparing substituted phenylcyclopropylamine derivatives with high yields and purity, to resolve the problems associated with the processes described in the prior art, and that will be suitable for large-scale preparation. Furthermore, there remains a need for novel acid addition salts of trans-(1R, 2S)-2-(3,4-difluorophenyl)-cyclopropylamine and use thereof for preparing highly pure ticagrelor or a pharmaceutically acceptable salt thereof. Desirable process properties include non-hazardous conditions, environmentally friendly and easy to handle reagents, reduced reaction times, reduced cost, greater simplicity, increased purity, and increased yield of the product, thereby enabling the production of triazolo[4, 5-d]pyrimidinecyclopentane compounds, preferably ticagrelor, and their pharmaceutically acceptable acid addition salts in high purity and with high yield. The present invention satisfies this need and provides related advantages as well.

### BRIEF SUMMARY OF THE INVENTION

In a first aspect, the inventions provides novel, efficient, industrially advantageous and environmentally friendly methods for the preparation of substituted phenylcyclopropylamine derivatives using novel intermediates, preferably trans-(1R, 2S)-2-(3,4-difluorophenyl)-cyclopropylamine or an acid addition salt thereof, in high yield, and with high chemical and enantiomeric purity. Moreover, the methods disclosed herein involve non-hazardous and easy to handle catalysts and reagents, reduced reaction times, and reduced synthesis steps. The methods avoid the tedious and cumbersome procedures of the prior methods and are convenient to operate on a commercial scale.

In another aspect, the present disclosure also encompasses the use of pure trans-(1R,2S)-2-(3,4-difluorophenyl)-cyclopropylamine or an acid addition salt thereof obtained by the methods disclosed herein for preparing ticagrelor or a pharmaceutically acceptable salt thereof.

The method for the preparation of substituted phenylcyclopropylamine derivatives disclosed herein has the following advantages over the methods described in the prior art:

i) the overall method involves a reduced number of method steps and shorter reaction times;

ii) the method avoids the use of hazardous or explosive chemicals like sodium hydride, diazomethane, pyridine and sodium azide;

iii) the method avoids the use of tedious and cumbersome procedures like column chromatographic purifications and multiple isolations;

iv) the method avoids the use of expensive materials like chiral sultam auxiliary;

v) the method involves easy work-up methods and simple isolation methods, and there is a reduction in chemical waste;

vi) the purity of the product is increased without additional purifications; and

vii) the overall yield of the product is increased.

In some embodiments, the method includes incubating an olefinic substrate and a diazoketone reagent or a diazoester reagent with a cyclopropanation catalyst such as a heme enzyme to form a cyclopropane product. In some embodiments, the cyclopropane product is trans-(1R,2S)-2-(3,4-difluorophenyl)-cyclopropylamine. In other embodiments, the cyclopropane product is converted to trans-(1R,2S)-2-(3,4-difluorophenyl)-cyclopropylamine in one or more synthetic steps.

In particular embodiments, the present invention provides a method for the biocatalytic synthesis of trans-(1R,2S)-2-(3,4-difluorophenyl)-cyclopropylamine catalyzed by a heme enzyme such as a cytochrome P450 enzyme (e.g., P450 BM3 enzyme) using a diazoketone and the Beckmann rearrangement as shown in Scheme 5.

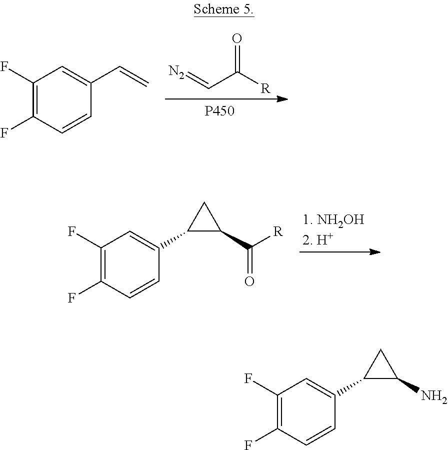

Other objects, features, and advantages of the present invention will be apparent to one of skill in the art from the following detailed description.

### BRIEF DESCRIPTION OF THE DRAWINGS

FIG. 1 shows the results of cyclopropanation reactions using M. infernorum hemoglobin variants for synthesis of (1R,2R)-2-(3,4-difluorophenyl)-1-cyclopropane-carboxylic acid ethyl ester.

FIG. 2 shows the results of cyclopropanation reactions using B. subtilis truncated hemoglobin variants for synthesis of (1R,2R)-2-(3,4-difluorophenyl)-1-cyclopropane-carboxylic acid ethyl ester.

### DETAILED DESCRIPTION OF THE INVENTION

### I. Introduction

The present disclosure relates to novel methods for the preparation of phenylcyclopropylamine derivatives, which are useful intermediates in the preparation of triazolo [4,5-d]pyrimidine compounds. The present disclosure particularly relates to novel, commercially viable and industrially advantageous methods for the preparation of a substantially pure ticagrelor intermediate, trans-(1R,2S)-2-(3,4-difluorophenyl)-cyclopropylamine. The intermediate is useful for preparing ticagrelor, or a pharmaceutically acceptable salt thereof, in high yield and purity. Because the biocatalytic cyclopropanation route uses an enzyme to set the chirality of the cyclopropane, the method of the present invention obviates the need to use chiral auxiliaries or chromatographic separation. Additionally, the method described herein requires only simple reagents, thus affording the cyclopropane product at a lower cost than published alternatives.

One of the published methods for making trans-(1R,2S)-2-(3,4-difluorophenyl)-cyclopropylamine using conventional chemistry methods is shown in Scheme 6. See, Owen et al., Drugs Fut., 32(10):845-853 (2007). This method for making trans-(1R,2S)-2-(3,4-difluorophenyl)-cyclopropylamine employs conventional chemistry that requires seven steps. In contrast, the present invention provides a low-cost and environmentally friendly synthesis of this key cyclopropane intermediate of ticagrelor by advantageously reducing the number of synthetic steps from 7 to 2, thus offering a substantial economic advantage.

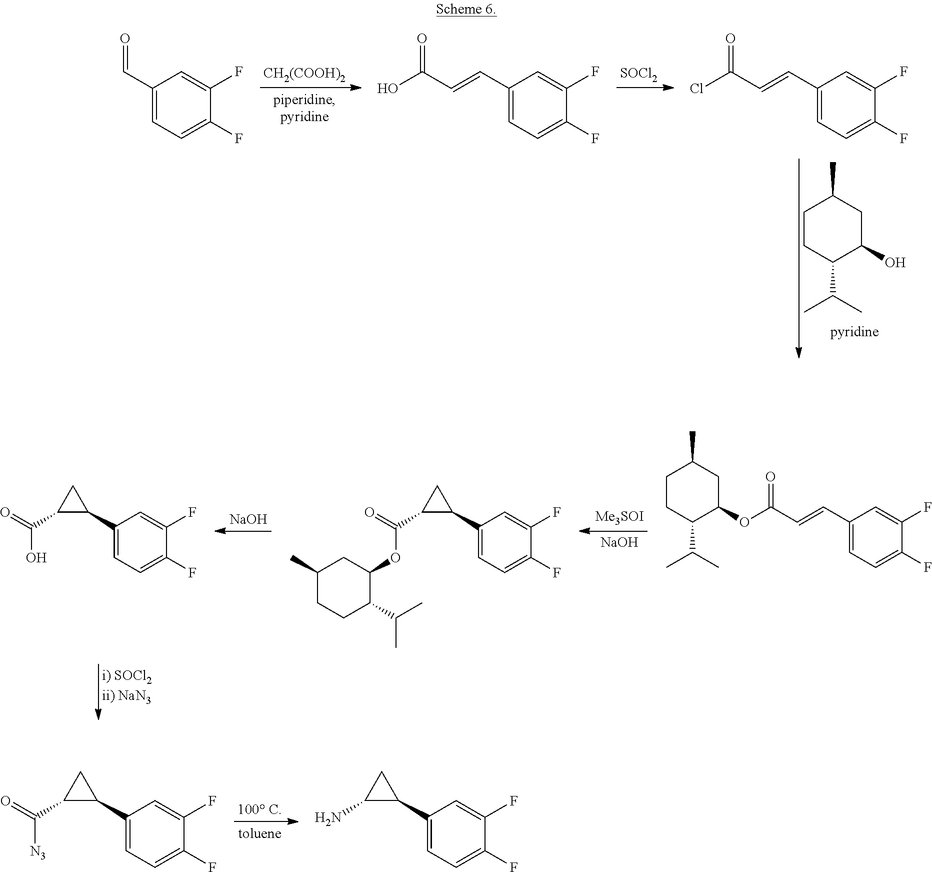

### II. Definitions

The following definitions and abbreviations are to be used for the interpretation of the invention. The term “invention” or “present invention” as used herein is a non-limiting term and is not intended to refer to any single embodiment but encompasses all possible embodiments.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

The terms “about” and “around,” as used herein to modify a numerical value, indicate a close range surrounding that explicit value. If “X” were the value, “about X” or “around X” would indicate a value from 0.9X to 1.TX, and more preferably, a value from 0.95X to 1.05X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

The term “cyclopropanation (enzyme) catalyst” or “enzyme with cyclopropanation activity” refers to any and all chemical processes catalyzed by enzymes, by which substrates containing at least one carbon-carbon double bond can be converted into cyclopropane products by using diazo reagents as carbene precursors.

The terms “engineered heme enzyme” and “heme enzyme variant” include any heme-containing enzyme comprising at least one amino acid mutation with respect to wild-type and also include any chimeric protein comprising recombined sequences or blocks of amino acids from two, three, or more different heme-containing enzymes.

The terms “engineered cytochrome P450” and “cytochrome P450 variant” include any cytochrome P450 enzyme comprising at least one amino acid mutation with respect to wild-type and also include any chimeric protein comprising recombined sequences or blocks of amino acids from two, three, or more different cytochrome P450 enzymes.

The term “whole cell catalyst” includes microbial cells expressing heme-containing enzymes, wherein the whole cell catalyst displays cyclopropanation activity.

As used herein, the terms “porphyrin” and “metal-substituted porphyrins” include any porphyrin that can be bound by a heme enzyme or variant thereof. In particular embodiments, these porphyrins may contain metals including, but not limited to, Fe, Mn, Co, Cu, Rh, and Ru.

The terms “carbene equivalent” and “carbene precursor” include molecules that can be decomposed in the presence of metal (or enzyme) catalysts to structures that contain at least one divalent carbon with only 6 valence shell electrons and that can be transferred to C═C bonds to form cyclopropanes or to C—H or heteroatom-H bonds to form various carbon ligated products.

The terms “carbene transfer” and “formal carbene transfer” as used herein include any chemical transformation where carbene equivalents are added to C═C bonds, carbon-heteroatom double bonds or inserted into C—H or heteroatom-H substrates.

As used herein, the terms “microbial,” “microbial organism” and “microorganism” include any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. Also included are cell cultures of any species that can be cultured for the production of a chemical.

As used herein, the term “non-naturally occurring”, when used in reference to a microbial organism or enzyme activity of the invention, is intended to mean that the microbial organism or enzyme has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary non-naturally occurring microbial organism or enzyme activity includes the cyclopropanation activity described above.

As used herein, the term “anaerobic”, when used in reference to a reaction, culture or growth condition, is intended to mean that the concentration of oxygen is less than about μM, preferably less than about 5 μM, and even more preferably less than 1 μM. The term is also intended to include sealed chambers of liquid or solid medium maintained with an atmosphere of less than about 1% oxygen. Preferably, anaerobic conditions are achieved by sparging a reaction mixture with an inert gas such as nitrogen or argon.

As used herein, the term “exogenous” is intended to mean that the referenced molecule or the referenced activity is introduced into the host microbial organism. The term as it is used in reference to expression of an encoding nucleic acid refers to the introduction of the encoding nucleic acid in an expressible form into the microbial organism. When used in reference to a biosynthetic activity, the term refers to an activity that is introduced into the host reference organism.

The term “heterologous” as used herein with reference to molecules, and in particular enzymes and polynucleotides, indicates molecules that are expressed in an organism other than the organism from which they originated or are found in nature, independently of the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently of the level of expression that can be lower equal or higher than the level of expression of the molecule in the native microorganism. It is understood that expression of native enzymes or polynucleotides may be modified in recombinant microorganisms.

The term “homolog,” as used herein with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Homologs most often have functional, structural, or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. Thus, the term “homologous proteins” is intended to mean that the two proteins have similar amino acid sequences. In particular embodiments, the homology between two proteins is indicative of its shared ancestry, related by evolution.

The terms “analog” and “analogous” include nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can refer to alkyl groups having up to 20 carbons atoms, such as, but not limited to heptyl, octyl, nonyl, decyl, etc. In some embodiments, alkyl groups have 1 to 12 carbon atoms. Alkyl groups can be optionally substituted with one or more moieties selected from halo, hydroxy, amino, alkylamino, alkoxy, haloalkyl, carboxy, amido, nitro, oxo, and cyano.

As used herein, the term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be optionally substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be optionally substituted with one or more moieties selected from halo, hydroxy, amino, alkylamino, alkoxy, haloalkyl, carboxy, amido, nitro, oxo, and cyano.

As used herein, the term “aryl” refers to an aromatic carbon ring system having any suitable number of ring atoms and any suitable number of rings. Aryl groups can include any suitable number of carbon ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be optionally substituted with one or more moieties selected from halo, hydroxy, amino, alkylamino, alkoxy, haloalkyl, carboxy, amido, nitro, oxo, and cyano.

As used herein, the term “aralkyl” denotes an arylalkyl group wherein the aryl and alkyl are as herein described. Preferred aralkyls contain a lower alkyl moiety. Exemplary aralkyl groups include benzyl, 2-phenethyl and naphthalenemethyl.

As used herein, the term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, and C₆₋₈. In some embodiments, cycloalkyl groups include 3 to 10 carbon atoms in the ring assembly. In some embodiments, cycloalkyl groups contain 5 to 10 carbon atoms in the ring assembly. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbomadiene. Cycloalkyl groups can be optionally substituted with one or more moieties selected from halo, hydroxy, amino, alkylamino, alkoxy, haloalkyl, carboxy, amido, nitro, oxo, and cyano.

As used herein, the term “heterocyclyl” refers to a saturated ring system having from 3 to 12 ring members and from 1 to 4 heteroatoms selected from N, O and S. Additional heteroatoms including, but not limited to, B, Al, Si and P can also be present in a heterocycloalkyl group. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 4 to 6, or 4 to 7 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. Examples of heterocyclyl groups include, but are not limited to, aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocyclyl groups can be optionally substituted with one or more moieties selected from halo, hydroxy, amino, alkylamino, alkoxy, haloalkyl, carboxy, amido, nitro, oxo, and cyano.

As used herein, the term “heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms including, but not limited to, B, Al, Si and P can also be present in a heteroaryl group. The heteroatoms can be oxidized to form moieties such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. Examples of heteroaryl groups include, but are not limited to, pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. Heteroaryl groups can be optionally substituted with one or more moieties selected from halo, hydroxy, amino, alkylamino, alkoxy, haloalkyl, carboxy, amido, nitro, oxo, and cyano.

As used herein, the term “alkoxy” refers to an alkyl group having an oxygen atom that connects the alkyl group to the point of attachment: i.e., alkyl-O—. As for alkyl group, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups can be optionally substituted with one or more moieties selected from halo, hydroxy, amino, alkylamino, alkoxy, haloalkyl, carboxy, amido, nitro, oxo, and cyano.

As used herein, the term “alkylthio” refers to an alkyl group having a sulfur atom that connects the alkyl group to the point of attachment: i.e., alkyl-S—. As for alkyl groups, alkylthio groups can have any suitable number of carbon atoms, such as C₁₋₆ or C₁₋₄. Alkylthio groups include, for example, methoxy, ethoxy, propoxy, iso-propoxy, butoxy, 2-butoxy, iso-butoxy, sec-butoxy, tert-butoxy, pentoxy, hexoxy, etc. Alkylthio groups can be optionally substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “haloalkyl” refers to an alkyl moiety as defined above substituted with at least one halogen atom.

As used herein, the term “alkylsilyl” refers to a moiety —SiR₃, wherein at least one R group is alkyl and the other R groups are H or alkyl. The alkyl groups can be substituted with one more halogen atoms.

As used herein, the term “acyl” refers to a moiety —C(O)R, wherein R is an alkyl group.

As used herein, the term “oxo” refers to an oxygen atom that is double-bonded to a compound (i.e., O═).

As used herein, the term “carboxy” refers to a moiety —C(O)OH. The carboxy moiety can be ionized to form the carboxylate anion.

As used herein, the term “amino” refers to a moiety —NR₃, wherein each R group is H or alkyl.

As used herein, the term “amido” refers to a moiety —NRC(O)R or —C(O)NR₂, wherein each R group is H or alkyl.

### III. Description of the Embodiments

The present invention provides a method by which trans-(1R,2S)-2-(3,4-difluorophenyl)-cyclopropylamine is prepared using synthetic strategies that are enabled by a biocatalytic step. The method includes incubating an olefinic substrate and a diazoester reagent or a diazoketone reagent with a cyclopropanation catalyst such as a heme enzyme to form a cyclopropane product. In some embodiments, the cyclopropane product is trans-(1R,2S)-2-(3,4-difluorophenyl)-cyclopropylamine. In other embodiments, the cyclopropane product is converted to trans-(1R,2S)-2-(3,4-difluorophenyl)-cyclopropylamine in one or more synthetic steps.

Accordingly, one aspect of the invention provides a method for producing a cyclopropanation product of Formula A:

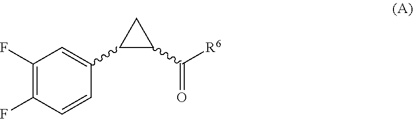

wherein R⁶ is selected from the group consisting of C₁₋₁₈ alkyl, C₁₋₁₈ alkenyl, C₁₋₁₈ alkynyl, C₁₋₁₈ alkoxy, C₁₋₁₈ alkenyloxy, C₁₋₁₈ alkynyloxy. The method includes combining an olefinic substrate, a carbene precursor, and a heme enzyme under conditions sufficient to form the product of Formula A. In some embodiments, R⁶ is C₁₋₁₈ alkoxy and the carbene precursor is a diazoester. In some embodiments, R⁶ is selected from the group consisting of C₁₋₁₈ alkyl, C₁₋₁₈ alkenyl, and C₁₋₁₈ alkynyl and the carbene precursor is a diazoketone.

In a related aspect, the invention provides a reaction mixture for producing a cyclopropanation product of Formula A. The reaction mixture includes an olefinic substrate, a carbene precursor, and a heme enzyme as described above.

In some embodiments, the invention provides a method for preparing substituted phenylcyclopropylamine derivatives of Formula XLII:

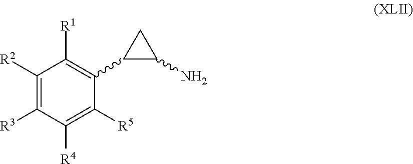

or a stereochemically isomeric form or a mixture of stereochemically isomeric forms thereof, or an acid addition salt thereof, wherein R¹, R², R³, R⁴, and R⁵ are, each independently, selected from hydrogen and a halogen atom, with the proviso that the benzene ring is substituted with at least one or more halogen atoms, wherein the halogen atom is F, Cl, Br or I, preferably, the halogen atom is F. The method includes:
a) reacting a halogen substituted benzaldehyde compound of Formula XLIII:

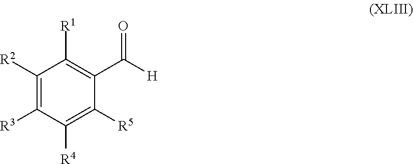

wherein R¹, R², R³, R⁴, and R⁵ are as defined in Formula XLII; with a methyltriphenyl phosphonium halide (Wittig reagent) of Formula XLIV:

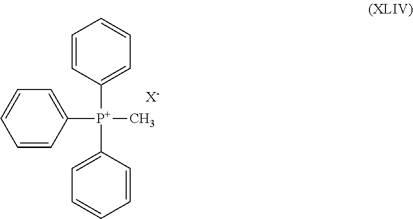

wherein X is a halogen, selected from the group consisting of Cl, Br and I;
in the presence of a first base in a first solvent to produce a substituted styrene compound of Formula XLV:

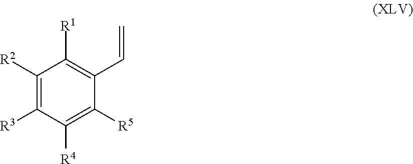

wherein R¹, R², R³, R⁴, and R⁵ are as defined above;
b) reacting the compound of Formula XLV with a diazoester compound of Formula XLVI:

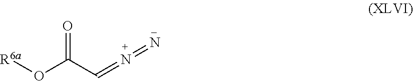

wherein R^{6a} is an alkyl, cycloalkyl, aryl or aralkyl group; in the presence of a heme protein catalyst to produce a substituted cyclopropanecarboxylate compound of Formula XLVIIa:

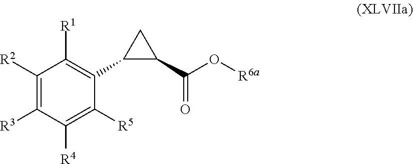

or a stereochemically isomeric form or a mixture of stereochemically isomeric forms thereof, wherein R¹, R², R³, R⁴, and R⁵ are as defined above;
c) hydrolyzing the ester compound of Formula XLVIIa with an acid or a second base in a third solvent to produce a substituted cyclopropanecarboxylic acid compound of Formula XLVIIIa:

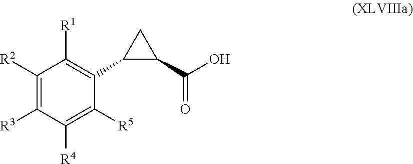

or a stereochemically isomeric form or a mixture of stereochemically isomeric forms thereof;
d) optionally, purifying the cyclopropanecarboxylic acid compound of Formula XLVIIIa by treating with a chiral amine in a fourth solvent to produce a pure chiral amine salt of the compound of Formula XLVIIIa;
e) optionally, acidifying the chiral amine salt of the compound of Formula XLVIIIa with an acid to produce a pure cyclopropanecarboxylic acid compound of Formula XLVIIIa;
f) reacting the cyclopropanecarboxylic acid compound of Formula XLVIIIa or a chiral amine salt thereof obtained in step-(c), (d) or (e) with an azide compound, with the proviso that the azide does not include sodium azide, in the presence a third base in a fifth solvent to produce an isocyanate intermediate, followed by subjecting to acidic hydrolysis with an acid in a sixth solvent and then basifying with a fourth base to produce the substituted phenylcyclopropylamine derivatives of Formula XLII or a stereochemically isomeric form or a mixture of stereochemically isomeric forms thereof, and optionally converting the compound of Formula XLII obtained into an acid addition salt thereof.

In some embodiments, the halogen atom X in the compound of Formula XLIV is Cl or Br, and more specifically, X is Br.

In some embodiments, in the compounds of Formulae XLII, XLIII, XLV, XLVIIa, and XLVIIIa, the R¹, R⁴ and R⁵ are H, and the R² and R³ are F.

The compounds can exist in different isomeric forms such as cis/trans isomers, enantiomers, or diastereomers. The method disclosed herein includes all such isomeric forms and mixtures thereof in all proportions.

In some embodiments, the group R^{6a} in the compounds of Formulae XLVI and XLVIIa is selected from the group consisting of methyl, ethyl, isopropyl, tert-butyl, benzyl, L- or D-menthyl, and the like; and more specifically, R is ethyl.

In some embodiments, a specific substituted phenylcyclopropylamine derivative prepared by the methods described herein is trans-(1R, 2S)-2-(3,4-difluorophenyl)-cyclopropylamine of Formula IIa:

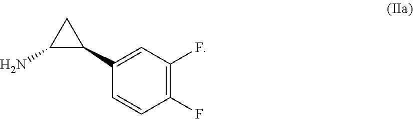 [SMILES: N[C@@H]1C[C@H]1c1ccc(F)c(F)c1]

In some embodiments, a specific substituted phenylcyclopropylamine derivative prepared by the methods described herein is trans-(1S,2R)-2-(3,4-difluorophenyl)-cyclopropylamine of Formula IIb:

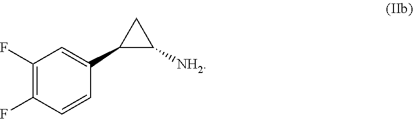 [SMILES: N[C@H]1C[C@@H]1c1ccc(F)c(F)c1]

In some instances, the heme enzyme variant produces a plurality of cyclopropanation products having a plurality of the E cyclopropane products. Moreover, in some instances the heme enzymes described herein produce a plurality of the desired 1R, 2R cyclopropane carboxylate product of Formula VIIa with a % ee of 20% or greater.

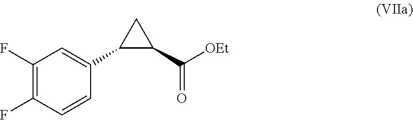 [SMILES: CCOC(=O)[C@@H]1C[C@H]1c1ccc(F)c(F)c1]

One skilled in the art will understand that suitably produced cyclopropane of Formula VII of variable enantioenrichment can be subjected to a hydrolase, lipase, or esterase enzyme that selectively hydrolyzes the ethyl ester of a single diastereomer of Formula VII to give exclusively or predominantly cyclopropane of Formula VIIIa or salt thereof, as shown in Scheme 7.

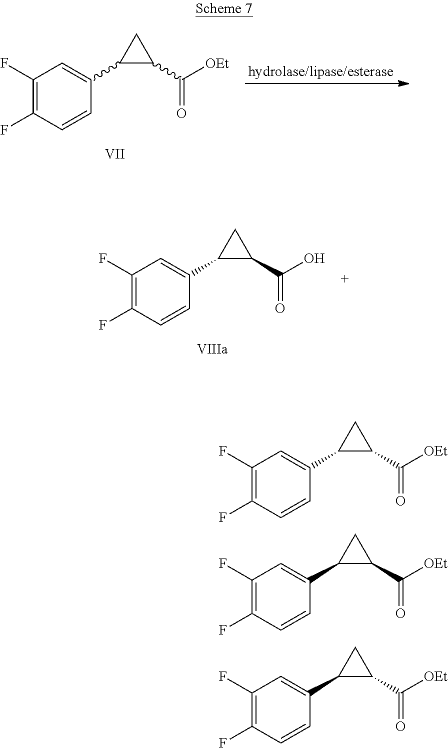

One skilled in the art will understand that suitably produced cyclopropane of Formula VII of variable enantioenrichment can be subjected to a hydrolase, lipase, or esterase enzyme that selectively maintains the ethyl ester of a single diastereomer of VII to give exclusively or predominantly cyclopropane carboxylic acid ethyl ester of Formula VIIa, as shown in Scheme 8.

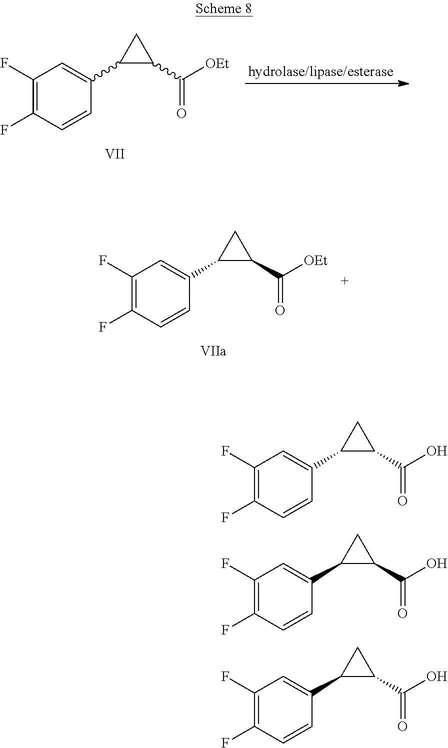

A lipase isolated from Thermomyces lanuginosus (ALMAC lipase kit; AH-45) catalyzes the transformation shown in Scheme 8, and can provide excellent levels of enantioenrichment of the desired enantiomer as the ethyl ester of Formula VIIa.

The cyclopropane carboxylate ethyl ester of Formula VIIa can be selectively removed from the reaction milieu shown in Scheme 8 by selective extraction or distillation or chromatographic separation. Subsequent chemical hydrolysis or enzymatic hydrolysis of the ethyl ester of Formula VIIa will then yield the desired enantiopure or enantioenriched cyclopropane carboxylate of Formula VIIIa, which can then be converted to the desired compound of Formula IIa.

A. Heme Enzymes

In general, methods of the invention include the use of one or more heme enzymes that catalyze the conversion of an olefinic substrate to products containing one or more cyclopropane functional groups. In some embodiments, the present invention provides methods which use heme enzyme variants comprising at least one or more amino acid mutations therein that catalyze the formal transfer of carbene equivalents from a diazo reagent (e.g., a diazoester or a diazoketone) to an olefinic substrate, making cyclopropane products with high stereoselectivity. In certain embodiments, the heme enzyme variants of the present invention have the ability to catalyze cyclopropanation reactions efficiently, display increased total turnover numbers, and/or demonstrate highly regio- and/or enantioselective product formation compared to the corresponding wild-type enzymes.

The terms “heme enzyme” and “heme protein” are used herein to include any member of a group of proteins containing heme as a prosthetic group. Non-limiting examples of heme enzymes include globins, cytochromes, oxidoreductases, any other protein containing a heme as a prosthetic group, and combinations thereof. Heme-containing globins include, but are not limited to, hemoglobin, myoglobin, and combinations thereof. Heme-containing cytochromes include, but are not limited to, cytochrome P450, cytochrome b, cytochrome c1, cytochrome c, and combinations thereof. Heme-containing oxidoreductases include, but are not limited to, a catalase, an oxidase, an oxygenase, a haloperoxidase, a peroxidase, and combinations thereof.

Exemplary catalysts used in the cyclopropanation reactions include hemoproteins of the sort described in U.S. Pat. No. 8,993,262. In certain embodiments, the catalyst is comprised of a natural or engineered hemoprotein containing a histidine at the axial position of the heme coordination site. In particular embodiments, the heme enzyme comprises a histidine mutation at the axial position of the heme coordination site.

In certain instances, the heme enzymes are metal-substituted heme enzymes containing protoporphyrin IX or other porphyrin molecules containing metals other than iron, including, but not limited to, cobalt, rhodium, copper, ruthenium, and manganese, which are active cyclopropanation catalysts.

In certain embodiments, mutations can be introduced into a target gene using standard cloning techniques (e.g., site-directed mutagenesis) or by gene synthesis to produce heme enzyme variants (e.g., cytochrome P450 variants). Heme enzyme variants can be expressed in a host cell (e.g., bacterial cell) using an expression vector under the control of an inducible promoter or by means of chromosomal integration under the control of a constitutive promoter. Cyclopropanation activity can be screened in vivo or in vitro by following product formation by GC or HPLC as described herein.

The expression vector comprising a nucleic acid sequence that encodes a heme enzyme of the invention can be a viral vector, a plasmid, a phage, a phagemid, a cosmid, a fosmid, a bacteriophage (e.g., a bacteriophage PI-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). Expression vectors can include chromosomal, non-chromosomal, and synthetic DNA sequences. Equivalent expression vectors to those described herein are known in the art and will be apparent to the ordinarily skilled artisan.

The expression vector can include a nucleic acid sequence encoding a heme enzyme that is operably linked to a promoter, wherein the promoter comprises a viral, bacterial, archaeal, fungal, insect, or mammalian promoter. In certain embodiments, the promoter is a constitutive promoter. In some embodiments, the promoter is an inducible promoter. In other embodiments, the promoter is a tissue-specific promoter or an environmentally regulated or a developmentally regulated promoter.

It is understood that affinity tags may be added to the N- and/or C-terminus of a heme enzyme expressed using an expression vector to facilitate protein purification. Non-limiting examples of affinity tags include metal binding tags such as His6-tags and other tags such as glutathione S-transferase (GST)

Non-limiting expression vectors for use in bacterial host cells include pCWori, pET vectors such as pET22 (EMD Millipore), pBR322 (ATCC37017), pQE™ vectors (Qiagen), pB!uescript™ vectors (Stratagene), pNH vectors, lambdaZAP vectors (Stratagene); ptrc99a, pKK223-3, pDR540, pRIT2T (Pharmacia), pRSET, pCR-TOPO vectors, pET vectors, pSyn_1 vectors, pChlamy I vectors (Life Technologies, Carlsbad, Calif.), pGEMI (Promega, Madison, Wis.), and pMAL (New England Biolabs, Ipswich, Mass.). Nonlimiting examples of expression vectors for use in eukaryotic host cells include pXT1, pSG5 (Stratagene), pSVK3, pBPV, pMSG, pSVLSV40 (Pharmacia), pcDNA3.3, pcDNA4/TO, pcDNA6/TR, pLenti6/TR, pMT vectors (Life Technologies), pKLACI vectors, pKLAC2 vectors (New England Biolabs), pQE™ vectors (Qiagen), BacPak baculoviral vectors, pAdeno-X™ adenoviral vectors (Clontech), and pBABE retroviral vectors. Any other vector may be used as long as it is replicable and viable in the host cell.

The host cell can be a bacterial cell, an archaeal cell, a fungal cell, a yeast cell, an insect cell, or a mammalian cell.

Suitable bacterial host cells include, but are not limited to, BL21 E. coli, DE3 strain E. coli, E. coli MIS, DH5a, DHIO˜, HBIOI, T7 Express Competent E. coli (NEB), B. subtilis cells, Pseudomonas fluorescens cells, and cyanobacterial cells such as Chlamydomonas reinhardtii cells and Synechococcus elongates cells. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. Fungal host cells include, but are not limited to, yeast cells from the genera Saccharomyces (e.g., S. cerevisiae), Pichia (P. Pastoris), Kluyveromyces (e.g., K. lac tis), Hansenula and Yarrowia, and filamentous fungal cells from the genera Aspergillus, Trichoderma, and Myceliophthora. Suitable insect host cells include, but are not limited to, Sf9 cells from Spodoptera frugiperda, Sj21 cells from Spodoptera frugiperda, Hi-Five cells, BT1-TN-5B 1-4 Trichophusia ni cells, and Schneider 2 (S2) cells and Schneider 3 (S3) cells from Drosophila melanogaster. Non-limiting examples of mammalian host cells include HEK293 cells, HeLa cells, CHO cells, COS cells, Jurkat cells, NSO hybridoma cells, baby hamster kidney (BHK) cells, MDCK cells, NIH-3T3 fibroblast cells, and any other immortalized cell line derived from a mammalian cell.

In certain embodiments, the present invention provides heme enzymes such as the P450 variants described herein that are active cyclopropanation catalysts inside living cells. As a non-limiting example, bacterial cells (e.g., E. coli) can be used as whole cell catalysts for the in vivo cyclopropanation reactions of the present invention.

One skilled in the art will appreciate that the hemoprotein catalysts described herein can be improved through the introduction of additional DNA mutations which alter the resulting amino acid sequence of the hemoprotein catalyst so as to generate a catalyst that is highly selective for the desired cyclopropane (for example, giving a % ee greater than 95%). In particular, there are many examples in the scientific literature that describe processes through which the enantioselectivity and activity of hemoprotein carbene-transfer catalysts can be optimized (Wang, Z. J. et al. Angew. Chem. Int. Ed. 2013, 52, 6928-6931; Heel T. et al. 2014, ChemBioChem, 15, 2556-2562; Coelho P. S. et al. Nat. Chem. Biol. 2013, 9, 485-487; Coelho P. S. et al. Science 2013, 339, 307-310). Specifically, one skilled in the art will know that through a process of random mutagenesis via error-prone PCR, or through a process of site-directed mutagenesis in which one or more codons are randomized sequentially or simultaneously, or through a process of gene synthesis in which random or directed mutations are introduced, many different mutants of the genes encoding the hemoprotein catalysts described herein can be generated.

One skilled in the art will understand that mutant genes encoding hemoprotein catalysts can be produced as hemoproteins in suitable hosts as described herein.

One skilled in the art will understand that suitably produced hemoprotein mutants can then be screened by various methods including but not limited to LC-MS, HPLC, GC, or SFC to determine whether one or several mutations introduced are beneficial for any desired parameter (% ee, % yield, specific activity, expression, solvent tolerance) that improves the hemoprotein-catalyzed synthesis of cyclopropanation products.

One skilled in the art will understand that hemoprotein mutants identified as improved in the synthesis of the cyclopropanation products can themselves be subjected to additional mutagenesis as described herein, resulting in progressive, cumulative improvements in one or more reaction parameters including but not limited to % ee, % yield, specific activity, expression, or solvent tolerance.

In some embodiments, the heme enzyme is a member of one of the enzyme classes set forth in Table 1. In other embodiments, the heme enzyme is a variant or homolog of a member of one of the enzyme classes set forth in Table 1. In yet other embodiments, the heme enzyme comprises or consists of the heme domain of a member of one of the enzyme classes set forth in Table 1 or a fragment thereof (e.g., a truncated heme domain) that is capable of carrying out the cyclopropanation reactions described herein.

TABLE 1
Heme enzymes identified by their enzyme classification
number (EC number) and classification name.

| EC Number | Name |
|---|---|
| 1.1.2.3 | L-lactate dehydrogenase |
| 1.1.2.6 | polyvinyl alcohol dehydrogenase (cytochrome) |
| 1.1.2.7 | methanol dehydrogenase (cytochrome c) |
| 1.1.5.5 | alcohol dehydrogenase (quinone) |
| 1.1.5.6 | formate dehydrogenase-N: |
| 1.1.9.1 | alcohol dehydrogenase (azurin): |
| 1.1.99.3 | gluconate 2-dehydrogenase (acceptor) |
| 1.1.99.11 | fructose 5-dehydrogenase |
| 1.1.99.18 | cellobiose dehydrogenase (acceptor) |
| 1.1.99.20 | alkan-1-ol dehydrogenase (acceptor) |
| 1.2.1.70 | glutamyl-tRNA reductase |
| 1.2.3.7 | indole-3-acetaldehyde oxidase |
| 1.2.99.3 | aldehyde dehydrogenase (pyrroloquinoline-quinone) |
| 1.3.1.6 | fumarate reductase (NADH): |
| 1.3.5.1 | succinate dehydrogenase (ubiquinone) |
| 1.3.5.4 | fumarate reductase (menaquinone) |
| 1.3.99.1 | succinate dehydrogenase |
| 1.4.9.1 | methylamine dehydrogenase (amicyanin) |
| 1.4.9.2. | aralkylamine dehydrogenase (azurin) |
| 1.5.1.20 | methylenetetrahydrofolate reductase [NAD(P)H] |
| 1.5.99.6 | spermidine dehydrogenase |
| 1.6.3.1 | NAD(P)H oxidase |
| 1.7.1.1 | nitrate reductase (NADH) |
| 1.7.1.2 | Nitrate reductase [NAD(P)H] |
| 1.7.1.3 | nitrate reductase (NADPH) |
| 1.7.1.4 | nitrite reductase [NAD(P)H] |
| 1.7.1.14 | nitric oxide reductase [NAD(P), nitrous oxide-forming] |
| 1.7.2.1 | nitrite reductase (NO-forming) |
| 1.7.2.2 | nitrite reductase (cytochrome; ammonia-forming) |
| 1.7.2.3 | trimethylamine-N-oxide reductase (cytochrome c) |
| 1.7.2.5 | nitric oxide reductase (cytochrome c) |
| 1.7.2.6 | hydroxylamine dehydrogenase |
| 1.7.3.6 | hydroxylamine oxidase (cytochrome) |
| 1.7.5.1 | nitrate reductase (quinone) |
| 1.7.5.2 | nitric oxide reductase (menaquinol) |
| 1.7.6.1 | nitrite dismutase |
| 1.7.7.1 | ferredoxin-nitrite reductase |
| 1.7.7.2 | ferredoxin-nitrate reductase |
| 1.7.99.4 | nitrate reductase |
| 1.7.99.8 | hydrazine oxidoreductase |
| 1.8.1.2 | sulfite reductase (NADPH) |
| 1.8.2.1 | sulfite dehydrogenase |
| 1.8.2.2 | thiosulfate dehydrogenase |
| 1.8.2.3 | sulfide-cytochrome-c reductase (flavocytochrome c) |
| 1.8.2.4 | dimethyl sulfide:cytochrome c2 reductase |
| 1.8.3.1 | sulfite oxidase |
| 1.8.7.1 | sulfite reductase (ferredoxin) |
| 1.8.98.1 | CoB-CoM heterodisulfide reductase |
| 1.8.99.1 | sulfite reductase |
| 1.8.99.2 | adenylyl-sulfate reductase |
| 1.8.99.3 | hydrogensulfite reductase |
| 1.9.3.1 | cytochrome-c oxidase |
| 1.9.6.1 | nitrate reductase (cytochrome) |
| 1.10.2.2 | ubiquinol-cytochrome-c reductase |
| 1.10.3.1 | catechol oxidase |
| 1.10.3.B1 | caldariellaquinol oxidase (H+-transporting) |
| 1.10.3.3 | L-ascorbate oxidase |
| 1.10.3.9 | photosystem II |
| 1.10.3.10 | ubiquinol oxidase (H+-transporting) |
| 1.10.3.11 | ubiquinol oxidase |
| 1.10.3.12 | menaquinol oxidase (H+-transporting) |
| 1.10.9.1 | plastoquinol-plastocyanin reductase |
| 1.11.1.5 | cytochrome-c peroxidase |
| 1.11.1.6 | catalase |
| 1.11.1.7 | peroxidase |
| 1.11.1.B2 | chloride peroxidase (vanadium-containing) |
| 1.11.1.B7 | bromide peroxidase (heme-containing) |
| 1.11.1.8 | iodide peroxidase |
| 1.11.1.10 | chloride peroxidase |
| 1.11.1.11 | L-ascorbate peroxidase |
| 1.11.1.13 | manganese peroxidase |
| 1.11.1.14 | lignin peroxidase |
| 1.11.1.16 | versatile peroxidase |
| 1.11.1.19 | dye decolorizing peroxidase |
| 1.11.1.21 | catalase-peroxidase |
| 1.11.2.1 | unspecific peroxygenase |
| 1.11.2.2 | myeloperoxidase |
| 1.11.2.3 | plant seed peroxygenase |
| 1.11.2.4 | fatty-acid peroxygenase |
| 1.12.2.1 | cytochrome-c3 hydrogenase |
| 1.12.5.1 | hydrogen:quinone oxidoreductase |
| 1.12.99.6 | hydrogenase (acceptor) |
| 1.13.11.9 | 2,5-dihydroxypyridine 5,6-dioxygenase |
| 1.13.11.11 | tryptophan 2,3-dioxygenase |
| 1.13.11.49 | chlorite O2-lyase |
| 1.13.11.50 | acetylacetone-cleaving enzyme |
| 1.13.11.52 | indoleamine 2,3-dioxygenase |
| 1.13.11.60 | linoleate 8R-lipoxygenase |
| 1.13.99.3 | tryptophan 2′-dioxygenase |
| 1.14.11.9 | flavanone 3-dioxygenase |
| 1.14.12.17 | nitric oxide dioxygenase |
| 1.14.13.39 | nitric-oxide synthase (NADPH dependent) |
| 1.14.13.17 | cholesterol 7alpha-monooxygenase |
| 1.14.13.41 | tyrosine N-monooxygenase |
| 1.14.13.70 | sterol 14alpha-demethylase |
| 1.14.13.71 | N-methylcoclaurine 3′-monooxygenase |
| 1.14.13.81 | magnesium-protoporphyrin IX monomethyl ester |
| | (oxidative) cyclase |
| 1.14.13.86 | 2-hydroxyisoflavanone synthase |
| 1.14.13.98 | cholesterol 24-hydroxylase |
| 1.14.13.119 | 5-epiaristolochene 1,3-dihydroxylase |
| 1.14.13.126 | vitamin D3 24-hydroxylase |
| 1.14.13.129 | beta-carotene 3-hydroxylase |
| 1.14.13.141 | cholest-4-en-3-one 26-monooxygenase |
| 1.14.13.142 | 3-ketosteroid 9alpha-monooxygenase |
| 1.14.13.151 | linalool 8-monooxygenase |
| 1.14.13.156 | 1,8-cineole 2-endo-monooxygenase |
| 1.14.13.159 | vitamin D 25-hydroxylase |
| 1.14.14.1 | unspecific monooxygenase |
| 1.14.15.1 | camphor 5-monooxygenase |
| 1.14.15.6 | cholesterol monooxygenase (side-chain-cleaving) |
| 1.14.15.8 | steroid 15beta-monooxygenase |
| 1.14.15.9 | spheroidene monooxygenase |
| 1.14.18.1 | tyrosinase |
| 1.14.19.1 | stearoyl-CoA 9-desaturase |
| 1.14.19.3 | linoleoyl-CoA desaturase |
| 1.14.21.7 | biflaviolin synthase |
| 1.14.99.1 | prostaglandin-endoperoxide synthase |
| 1.14.99.3 | heme oxygenase |
| 1.14.99.9 | steroid 17alpha-monooxygenase |
| 1.14.99.10 | steroid 21-monooxygenase |
| 1.14.99.15 | 4-methoxybenzoate monooxygenase (O-demethylating) |
| 1.14.99.45 | carotene epsilon-monooxygenase |
| 1.16.5.1 | ascorbate ferrireductase (transmembrane) |
| 1.16.9.1 | iron:rusticyanin reductase |
| 1.17.1.4 | xanthine dehydrogenase |
| 1.17.2.2 | lupanine 17-hydroxylase (cytochrome c) |
| 1.17.99.1 | 4-methylphenol dehydrogenase (hydroxylating) |
| 1.17.99.2 | ethylbenzene hydroxylase |
| 1.97.1.1 | chlorate reductase |
| 1.97.1.9 | selenate reductase |
| 2.7.7.65 | diguanylate cyclase |
| 2.7.13.3 | histidine kinase |
| 3.1.4.52 | cyclic-guanylate-specific phosphodiesterase |
| 4.2.1.B9 | colneleic acid/etheroleic acid synthase |
| 4.2.1.22 | Cystathionine beta-synthase |
| 4.2.1.92 | hydroperoxide dehydratase |
| 4.2.1.212 | colneleate synthase |
| 4.3.1.26 | chromopyrrolate synthase |
| 4.6.1.2 | guanylate cyclase |
| 4.99.1.3 | sirohydrochlorin cobaltochelatase |
| 4.99.1.5 | aliphatic aldoxime dehydratase |
| 4.99.1.7 | phenylacetaldoxime dehydratase |
| 5.3.99.3 | prostaglandin-E synthase |
| 5.3.99.4 | prostaglandin-I synthase |
| 5.3.99.5 | Thromboxane-A synthase |
| 5.4.4.5 | 9,12-octadecadienoate 8-hydroperoxide 8R-isomerase |
| 5.4.4.6 | 9,12-octadecadienoate 8-hydroperoxide 8S-isomerase |
| 6.6.1.2 | cobaltochelatase |

In particular embodiments, the heme enzyme is a variant or a fragment thereof (e.g., a truncated variant containing the heme domain) comprising at least one mutation such as, e.g., a mutation at the axial position of the heme coordination site. In some instances, the mutation is a substitution of the native residue with Ala, Asp, Arg, Asn, Cys, Glu, Gln, Gly, His, Ile, Lys, Leu, Met, Phe, Pro, Ser, Thr, Trp, Tyr, or Val at the axial position. In certain instances, the mutation is a substitution of Cys with any other amino acid such as Ser at the axial position.

In certain embodiments, the in vitro methods for producing a cyclopropanation product comprise providing a heme enzyme, variant, or homolog thereof with a reducing agent such as NADPH or a dithionite salt (e.g., Na₂S₂O₄). In certain other embodiments, the in vivo methods for producing a cyclopropanation product comprise providing whole cells such as E. coli cells expressing a heme enzyme, variant, or homolog thereof.

In some embodiments, the heme enzyme, variant, or homolog thereof is recombinantly expressed and optionally isolated and/or purified for carrying out the in vitro cyclopropanation reactions of the present invention. In other embodiments, the heme enzyme, variant, or homolog thereof is expressed in whole cells such as E. coli cells, and these cells are used for carrying out the in vivo cyclopropanation reactions of the present invention.

In certain embodiments, the heme enzyme, variant, or homolog thereof comprises or consists of the same number of amino acid residues as the wild-type enzyme (e.g., a full-length polypeptide). In some instances, the heme enzyme, variant, or homolog thereof comprises or consists of an amino acid sequence without the start methionine (e.g., P450 BM3 amino acid sequence set forth in SEQ ID NO:1). In other embodiments, the heme enzyme comprises or consists of a heme domain fused to a reductase domain. In yet other embodiments, the heme enzyme does not contain a reductase domain, e.g., the heme enzyme contains a heme domain only or a fragment thereof such as a truncated heme domain.

In some embodiments, the heme enzyme, variant, or homolog thereof has an enhanced cyclopropanation activity of at least about 1.5, 2, 3, 4, 5, 6, 7, 8, 9, 10, 15, 20, 25, 30, 35, 40, 45, 50, 55, 60, 65, 70, 75, 80, 85, 90, 95, or 100 fold compared to the corresponding wild-type heme enzyme.

In some embodiments, the heme enzyme, variant, or homolog thereof has a resting state reduction potential higher than that of NADH or NADPH.

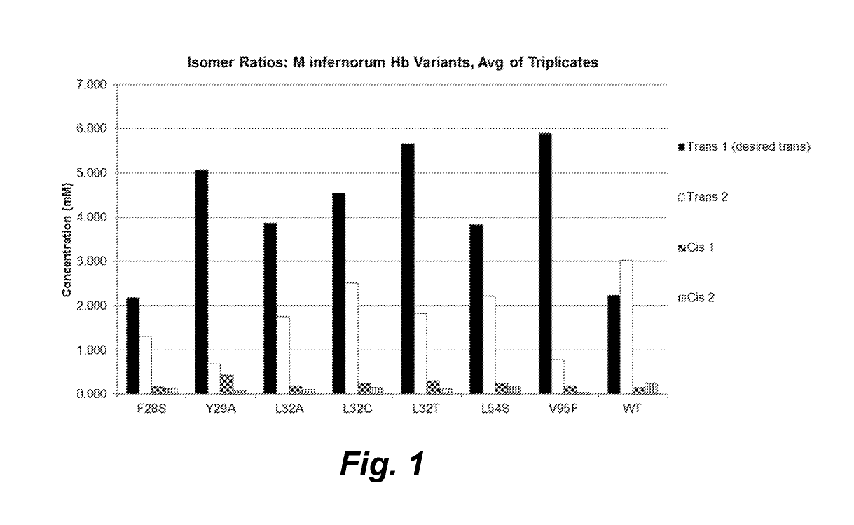

In particular embodiments, the heme enzyme comprises a cytochrome P450 enzyme. Cytochrome P450 enzymes constitute a large superfamily of heme-thiolate proteins involved in the metabolism of a wide variety of both exogenous and endogenous compounds. Usually, they act as the terminal oxidase in multicomponent electron transfer chains, such as P450-containing monooxygenase systems. Members of the cytochrome P450 enzyme family catalyze myriad oxidative transformations, including, e.g., hydroxylation, epoxidation, oxidative ring coupling, heteroatom release, and heteroatom oxygenation (E. M. Isin et al., Biochim. Biophys. Acta 1770, 314 (2007)). The active site of these enzymes contains an Fe^{III}-protoporphyrin IX cofactor (heme) ligated proximally by a conserved cysteine thiolate (M. T. Green, Current Opinion in Chemical Biology 13, 84 (2009)). The remaining axial iron coordination site is occupied by a water molecule in the resting enzyme, but during native catalysis, this site is capable of binding molecular oxygen. In the presence of an electron source, typically provided by NADH or NADPH from an adjacent fused reductase domain or an accessory cytochrome P450 reductase enzyme, the heme center of cytochrome P450 activates molecular oxygen, generating a high valent iron(IV)-oxo porphyrin cation radical species intermediate (Compound I, FIG. 1) and a molecule of water.

In certain embodiments the heme enzyme is a cytochrome P450 enzyme or a variant thereof. In a particular embodiment the cytochrome P450 enzyme is a P450 BM3 (also known as CYP102A1) enzyme or a variant thereof. In a further embodiment, the P450 BM3 enzyme comprises an axial ligand mutation C400H with or without an additional mutation at T268 to any other amino acid. In a further embodiment the P450 BM3 enzyme comprises a double mutant having an axial ligand mutation C400H and a further mutation of T268A. In certain embodiments, the CYP102A1 variants comprise the mutations T268A, C400H, L437W, V78M and L181V. These are termed BM3 Hstar and variants thereof, which are described in U.S. Pat. Appl. Pub. No. 2016/0032330 which is incorporated herein by reference in its entirety (see also, Angew. Chem. Int. Ed. 2014, 53, 6810-6813).

In certain embodiments the heme enzyme is a cytochrome P450 enzyme or a variant thereof. In a particular embodiment the cytochrome P450 enzyme is a variant of the P450 enzyme CYP119 (Swiss-Prot: Q55080.1) from Sulfolobus acidocaldarius DSM 639 that contains a mutation at the axial heme ligand (residue C317) to any other amino acid residue that is among the naturally occurring twenty amino acids. In a further embodiment, the CYP119 enzyme consists of a single mutation of the position T213A to any other amino acid. In a further embodiment, the CYP119 enzyme consists of a axial ligand mutation C317 to any other amino acid along with a mutation of the position T213 to any other amino acid.

In certain embodiments the heme enzyme is a variant of the P450 enzyme CYP119 that contains mutations at either or both position C317 and H315. Each of these positions may be mutated to any other amino acid.

In some embodiments, the heme enzyme is a cytochrome P450 holoenzyme or a variant thereof. In certain embodiments, the heme enzyme is a cytochrome P450 heme domain or a variant thereof.

One skilled in the art will appreciate that the cytochrome P450 enzyme superfamily has been compiled in various databases, including, but not limited to, the P450 homepage (available at http://dmelson.uthsc.edu/CytochromeP450.html; see also, D. R. Nelson, Hum. Genomics 4, 59 (2009)), the cytochrome P450 enzyme engineering database (available at http://www.cyped.uni-stuttgart.de/cgi-bin/CYPED5/index.pl; see also, D. Sirim et al., BMC Biochem 10, 27 (2009)), and the SuperCyp database (available at http://bioinformatics.charite.de/supercyp/; see also, S. Preissner et al., Nucleic Acids Res. 38, D237 (2010)), the disclosures of which are incorporated herein by reference in their entirety for all purposes.

In certain embodiments, the cytochrome P450 enzymes used in the methods of present the invention are members of one of the classes shown in Table 2 (see, http://www.icgeb.org/˜p450srv/P450enzymes.html, the disclosure of which is incorporated herein by reference in its entirety for all purposes).

TABLE 2
Cytochrome P450 enzymes classified by their EC
number, recommended name, and family/gene name.

| EC | Recommended name | Family/gene |
|---|---|---|
| 1.3.3.9 | secologanin synthase | CYP72A1 |
| 1.14.13.11 | trans-cinnamate 4-monooxygenase | CYP73 |
| 1.14.13.12 | benzoate 4-monooxygenase | CYP53 |
| 1.14.13.13 | calcidiol 1-monooxygenase | CYP27 |
| 1.14.13.15 | cholestanetriol 26-monooxygenase | CYP27 |
| 1.14.13.17 | cholesterol 7α-monooxygenase | CYP7 |
| 1.14.13.21 | flavonoid 3′-monooxygenase | CYP75 |
| 1.14.13.28 | 3,9-dihydroxypterocarpan 6a-monooxygenase | CYP93A1 |
| 1.14.13.30 | leukotriene-B₄ 20-monooxygenase | CYP4F |
| 1.14.13.37 | methyltetrahydroprotoberberine | CYP93A1 |
| | 14-monooxygenase | |
| 1.14.13.41 | tyrosine N-monooxygenase | CYP79 |
| 1.14.13.42 | hydroxyphenylacetonitrile 2-monooxygenase | — |
| 1.14.13.47 | (−)-limonene 3-monooxygenase | — |
| 1.14.13.48 | (−)-limonene 6-monooxygenase | — |
| 1.14.13.49 | (−)-limonene 7-monooxygenase | — |
| 1.14.13.52 | isoflavone 3′-hydroxylase | — |
| 1.14.13.53 | isoflavone 2′-hydroxylase | — |
| 1.14.13.55 | protopine 6-monooxygenase | — |
| 1.14.13.56 | dihydrosanguinarine 10-monooxygenase | — |
| 1.14.13.57 | dihydrochelirubine 12-monooxygenase | — |
| 1.14.13.60 | 27-hydroxycholesterol 7α-monooxygenase | — |
| 1.14.13.70 | sterol 14-demethylase | CYP51 |
| 1.14.13.71 | N-methylcoclaurine 3′-monooxygenase | CYP80B1 |
| 1.14.13.73 | tabersonine 16-hydroxylase | CYP71D12 |
| 1.14.13.74 | 7-deoxyloganin 7-hydroxylase | — |
| 1.14.13.75 | vinorine hydroxylase | — |
| 1.14.13.76 | taxane 10β-hydroxylase | CYP725A1 |
| 1.14.13.77 | taxane 13α-hydroxylase | CYP725A2 |
| 1.14.13.78 | ent-kaurene oxidase | CYP701 |
| 1.14.13.79 | ent-kaurenoic acid oxidase | CYP88A |
| 1.14.14.1 | unspecific monooxygenase | multiple |
| 1.14.15.1 | camphor 5-monooxygenase | CYP101 |
| 1.14.15.3 | alkane 1-monooxygenase | CYP4A |
| 1.14.15.4 | steroid 11β-monooxygenase | CYP11B |
| 1.14.15.5 | corticosterone 18-monooxygenase | CYP11B |
| 1.14.15.6 | cholesterol monooxygenase | CYP11A |
| | (side-chain-cleaving) | |
| 1.14.21.1 | (S)-stylopine synthase | — |
| 1.14.21.2 | (S)-cheilanthifoline synthase | — |
| 1.14.21.3 | berbamunine synthase | CYP80 |
| 1.14.21.4 | salutaridine synthase | — |
| 1.14.21.5 | (S)-canadine synthase | — |
| 1.14.99.9 | steroid 17α-monooxygenase | CYP17 |
| 1.14.99.10 | steroid 21-monooxygenase | CYP21 |
| 1.14.99.22 | ecdysone 20-monooxygenase | — |
| 1.14.99.28 | linalool 8-monooxygenase | CYP111 |
| 4.2.1.92 | hydroperoxide dehydratase | CYP74 |
| 5.3.99.4 | prostaglandin-I synthase | CYP8 |
| 5.3.99.5 | thromboxane-A synthase | CYP5 |

Table 3 below lists additional cyctochrome P450 enzymes that are suitable for use in the cyclopropanation reactions of the present invention. The accession numbers in Table 3 are incorporated herein by reference in their entirety for all purposes. The cytochrome P450 gene and/or protein sequences disclosed in the following patent documents are hereby incorporated by reference in their entirety for all purposes: WO 2013/076258; CN 103160521; CN 103223219; KR 2013081394; JP 5222410; WO 2013/073775; WO 2013/054890; WO 2013/048898; WO 2013/031975; WO 2013/064411; U.S. Pat. No. 8,361,769; WO 2012/150326, CN 102747053; CN 102747052; JP 2012170409; WO 2013/115484; CN 103223219; KR 2013081394; CN 103194461; JP 5222410; WO 2013/086499; WO 2013/076258; WO 2013/073775; WO 2013/064411; WO 2013/054890; WO 2013/031975; U.S. Pat. No. 8,361,769; WO 2012/156976; WO 2012/150326; CN 102747053; CN 102747052; US 20120258938; JP 2012170409; CN 102399796; JP 2012055274; WO 2012/029914; WO 2012/028709; WO 2011/154523; JP 2011234631; WO 2011/121456; EP 2366782; WO 2011/105241; CN 102154234; WO 2011/093185; WO 2011/093187; WO 2011/093186; DE 102010000168; CN 102115757; CN 102093984; CN 102080069; JP 2011103864; WO 2011/042143; WO 2011/038313; JP 2011055721; WO 2011/025203; JP 2011024534; WO 2011/008231; WO 2011/008232; WO 2011/005786; IN 2009DE01216; DE 102009025996; WO 2010/134096; JP 2010233523; JP 2010220609; WO 2010/095721; WO 2010/064764; US 20100136595; JP 2010051174; WO 2010/024437; WO 2010/011882; WO 2009/108388; US 20090209010; US 20090124515; WO 2009/041470; KR 2009028942; WO 2009/039487; WO 2009/020231; JP 2009005687; CN 101333520; CN 101333521; US 20080248545; JP 2008237110; CN 101275141; WO 2008/118545; WO 2008/115844; CN 101255408; CN 101250506; CN 101250505; WO 2008/098198; WO 2008/096695; WO 2008/071673; WO 2008/073498; WO 2008/065370; WO 2008/067070; JP 2008127301; JP 2008054644; KR 794395; EP 1881066; WO 2007/147827; CN 101078014; JP 2007300852; WO 2007/048235; WO 2007/044688; WO 2007/032540; CN 1900286; CN 1900285; JP 2006340611; WO 2006/126723; KR 2006029792; KR 2006029795; WO 2006/105082; WO 2006/076094; US 2006/0156430; WO 2006/065126; JP 2006129836; CN 1746293; WO 2006/029398; JP 2006034215; JP 2006034214; WO 2006/009334; WO 2005/111216; WO 2005/080572; US 2005/0150002; WO 2005/061699; WO 2005/052152; WO 2005/038033; WO 2005/038018; WO 2005/030944; JP 2005065618; WO 2005/017106; WO 2005/017105; US 20050037411; WO 2005/010166; JP 2005021106; JP 2005021104; JP 2005021105; WO 2004/113527; CN 1472323; JP 2004261121; WO 2004/013339; WO 2004/011648; DE 10234126; WO 2004/003190; WO 2003/087381; WO 2003/078577; US 20030170627; US 20030166176; US 20030150025; WO 2003/057830; WO 2003/052050; CN 1358756; US 20030092658; US 20030078404; US 20030066103; WO 2003/014341; US 20030022334; WO 2003/008563; EP 1270722; US 20020187538; WO 2002/092801; WO 2002/088341; US 20020160950; WO 2002/083868; US 20020142379; WO 2002/072758; WO 2002/064765; US 20020076777; US 20020076774; US 20020076774; WO 2002/046386; WO 2002/044213; US 20020061566; CN 1315335; WO 2002/034922; WO 2002/033057; WO 2002/029018; WO 2002/018558; JP 2002058490; US 20020022254; WO 2002/008269; WO 2001/098461; WO 2001/081585; WO 2001/051622; WO 2001/034780; CN 1271005; WO 2001/011071; WO 2001/007630; WO 2001/007574; WO 2000/078973; U.S. Pat. No. 6,130,077; JP 2000152788; WO 2000/031273; WO 2000/020566; WO 2000/000585; DE 19826821; JP 11235174; U.S. Pat. No. 5,939,318; WO 99/19493; WO 99/18224; U.S. Pat. No. 5,886,157; WO 99/08812; U.S. Pat. No. 5,869,283; JP 10262665; WO 98/40470; EP 776974; DE 19507546; GB 2294692; U.S. Pat. No. 5,516,674; JP 07147975; WO 94/29434; JP 06205685; JP 05292959; JP 04144680; DD 298820; EP 477961; SU 1693043; JP 01047375; EP 281245; JP 62104583; JP 63044888; JP 62236485; JP 62104582; and JP 62019084.

TABLE 3
Additional cytochrome P450 enzymes for use in the present invention.

| | | | SEQ |
|---|---|---|---|
| Species | Cyp No. | Accession No. | ID NO |
| Bacillus megaterium | 102A1 | AAA87602 | 1 |
| Bacillus megaterium | 102A1 | ADA57069 | 2 |
| Bacillus megaterium | 102A1 | ADA57068 | 3 |
| Bacillus megaterium | 102A1 | ADA57062 | 4 |
| Bacillus megaterium | 102A1 | ADA57061 | 5 |
| Bacillus megaterium | 102A1 | ADA57059 | 6 |
| Bacillus megaterium | 102A1 | ADA57058 | 7 |
| Bacillus megaterium | 102A1 | ADA57055 | 8 |
| Bacillus megaterium | 102A1 | ACZ37122 | 9 |
| Bacillus megaterium | 102A1 | ADA57057 | 10 |
| Bacillus megaterium | 102A1 | ADA57056 | 11 |
| Mycobacterium sp. HXN-1500 | 153A6 | CAH04396 | 12 |
| Tetrahymena thermophile | 5013C2 | ABY59989 | 13 |
| Nonomuraea dietziae | | AGE14547.1 | 14 |
| Homo sapiens | 2R1 | NP_078790 | 15 |
| Macca mulatta | 2R1 | NP_001180887.1 | 16 |
| Canis familiaris | 2R1 | XP_854533 | 17 |
| Mus musculus | 2R1 | AAI08963 | 18 |
| Bacillus halodurans C-125 | 152A6 | NP_242623 | 19 |
| Streptomyces parvus | aryC | AFM80022 | 20 |
| Pseudomonas putida | 101A1 | P00183 | 21 |
| Homo sapiens | 2D7 | AAO49806 | 22 |
| Rattus norvegicus | C27 | AAB02287 | 23 |
| Oryctolagus cuniculus | 2B4 | AAA65840 | 24 |
| Bacillus subtilis | 102A2 | O08394 | 25 |
| Bacillus subtilis | 102A3 | O08336 | 26 |
| B. megaterium DSM 32 | 102A1 | P14779 | 27 |
| B. cereus ATCC14579 | 102A5 | AAP10153 | 28 |
| B. licheniformis ATTC1458 | 102A7 | YP 079990 | 29 |
| B. thuringiensis serovar | X | YP 037304 | 30 |
| konkukian | | | |
| str.97-27 | | | |
| R. metallidurans CH34 | 102E1 | YP 585608 | 31 |
| A. fumigatus Af293 | 505X | EAL92660 | 32 |
| A. nidulans FGSC A4 | 505A8 | EAA58234 | 33 |
| A. oryzae ATCC42149 | 505A3 | Q2U4F1 | 34 |
| A. oryzae ATCC42149 | X | Q2UNA2 | 35 |
| F. oxysporum | 505A1 | Q9Y8G7 | 36 |
| G. moniliformis | X | AAG27132 | 37 |
| G. zeae PH1 | 505A7 | EAA67736 | 38 |
| G. zeae PH1 | 505C2 | EAA77183 | 39 |
| M. grisea 70-15 syn | 505A5 | XP 365223 | 40 |
| N. crassa OR74 A | 505A2 | XP 961848 | 41 |
| Oryza sativa* | 97A | | |
| Oryza sativa* | 97B | | |
| Oryza sativa | 97C | ABB47954 | 42 |

The start methionine (“M”) may be present or absent from these sequences.
*See, M. Z. Lv et al., Plant Cell Physiol., 53(6): 987-1002 (2012).

In certain embodiments, the present invention provides amino acid substitutions that efficiently remove monooxygenation chemistry from cytochrome P450 enzymes. This system permits selective enzyme-driven cyclopropanation chemistry without competing side reactions mediated by native P450 catalysis. The invention also provides P450-mediated catalysis that is competent for cyclopropanation chemistry but not able to carry out traditional P450-mediated monooxygenation reactions as ‘orthogonal’ P450 catalysis and respective enzyme variants as ‘orthogonal’ P450s. In some instances, orthogonal P450 variants comprise a single amino acid mutation at the axial position of the heme coordination site (e.g., a C400S mutation in the P450 BM3 enzyme) that alters the proximal heme coordination environment. Accordingly, the present invention also provides P450 variants that contain an axial heme mutation in combination with one or more additional mutations described herein to provide orthogonal P450 variants that show enriched diastereoselective and/or enantioselective product distributions. The present invention further provides a compatible reducing agent for orthogonal P450 cyclopropanation catalysis that includes, but is not limited to, NAD(P)H or sodium dithionite.

In particular embodiments, the cytochrome P450 enzyme is one of the P450 enzymes or enzyme classes set forth in Table 2 or 3. In some embodiments, the cytochrome P450 enzyme is a variant or homolog of one of the P450 enzymes or enzyme classes set forth in Table 2 or 3. In preferred embodiments, the P450 enzyme variant comprises a mutation at the conserved cysteine (Cys or C) residue of the corresponding wild-type sequence that serves as the heme axial ligand to which the iron in protoporphyrin IX is attached. As non-limiting examples, axial mutants of any of the P450 enzymes set forth in Table 2 or 3 can comprise a mutation at the axial position (“AxX”) of the heme coordination site, wherein “X” is selected from Ala, Asp, Arg, Asn, Glu, Gln, Gly, His, Ile, Lys, Leu, Met, Phe, Pro, Ser, Thr, Trp, Tyr, and Val.

In certain embodiments, the conserved cysteine residue in a cytochrome P450 enzyme of interest that serves as the heme axial ligand and is attached to the iron in protoporphyrin IX can be identified by locating the segment of the DNA sequence in the corresponding cytochrome P450 gene which encodes the conserved cysteine residue. In some instances, this DNA segment is identified through detailed mutagenesis studies in a conserved region of the protein (see, e.g., Shimizu et al., Biochemistry 27, 4138-4141, 1988). In other instances, the conserved cysteine is identified through crystallographic study (see, e.g., Poulos et al., J. Mol. Biol 195:687-700, 1987).

In situations where detailed mutagenesis studies and crystallographic data are not available for a cytochrome P450 enzyme of interest, the axial ligand may be identified through phylogenetic study. Due to the similarities in amino acid sequence between P450 enzymes, standard protein alignment algorithms may show a phylogenetic similarity between a P450 enzyme for which crystallographic or mutagenesis data exist and a new P450 enzyme for which such data do not exist. Thus, the polypeptide sequences of the present invention for which the heme axial ligand is known can be used as a “query sequence” to perform a search against a specific new cytochrome P450 enzyme of interest or a database comprising cytochrome P450 sequences to identify the heme axial ligand. Such analyses can be performed using the BLAST programs (see, e.g., Altschul et al., J Mol Biol. 215(3):403-10(1990)). Software for performing BLAST analyses publicly available through the National Center for Biotechnology Information (http://ncbi.nlm.nih.gov). BLASTP is used for amino acid sequences.

Exemplary parameters for performing amino acid sequence alignments to identify the heme axial ligand in a P450 enzyme of interest using the BLASTP algorithm include E value=10, word size=3, Matrix=Blosum62, Gap opening=11, gap extension=1, and conditional compositional score matrix adjustment. Those skilled in the art will know what modifications can be made to the above parameters, e.g., to either increase or decrease the stringency of the comparison and/or to determine the relatedness of two or more sequences.

In preferred embodiments, the cytochrome P450 enzyme is a cytochrome P450 BM3 enzyme or a variant, homolog, or fragment thereof. The bacterial cytochrome P450 BM3 from Bacillus megaterium is a water soluble, long-chain fatty acid monooxygenase. The native P450 BM3 protein is comprised of a single polypeptide chain of 1048 amino acids and can be divided into 2 functional subdomains (see, L. O. Narhi et al., J. Biol. Chem. 261, 7160 (1986)). An N-terminal domain, amino acid residues 1-472, contains the heme-bound active site and is the location for monooxygenation catalysis. The remaining C-terminal amino acids encompass a reductase domain that provides the necessary electron equivalents from NADPH to reduce the heme cofactor and drive catalysis. The presence of a fused reductase domain in P450 BM3 creates a self-sufficient monooxygenase, obviating the need for exogenous accessory proteins for oxygen activation (see, id.). It has been shown that the N-terminal heme domain can be isolated as an individual, well-folded, soluble protein that retains activity in the presence of hydrogen peroxide as a terminal oxidant under appropriate conditions (P. C. Cirino et al., Angew. Chem., Int. Ed. 42, 3299 (2003)).

In certain instances, the cytochrome P450 BM3 enzyme comprises or consists of the amino acid sequence set forth in SEQ ID NO: 1. In certain other instances, the cytochrome P450 BM3 enzyme is a natural variant thereof as described, e.g., in J. Y. Kang et al., AMB Express 1:1 (2011), wherein the natural variants are divergent in amino acid sequence from the wild-type cytochrome P450 BM3 enzyme sequence (SEQ ID NO:1) by up to about 5% (e.g., SEQ ID NOS:2-11).

In particular embodiments, the P450 BM3 enzyme variant comprises or consists of the heme domain of the wild-type P450 BM3 enzyme sequence (e.g., amino acids 1-463 of SEQ ID NO:1) and optionally at least one mutation as described herein. In other embodiments, the P450 BM3 enzyme variant comprises or consists of a fragment of the heme domain of the wild-type P450 BM3 enzyme sequence (SEQ ID NO: 1), wherein the fragment is capable of carrying out the cyclopropanation reactions of the present invention. In some instances, the fragment includes the heme axial ligand and at least one, two, three, four, or five of the active site residues.

In certain embodiments, the P450 BM3 enzyme variant comprises a mutation at the axial position (“AxX”) of the heme coordination site, wherein “X” is selected from Ala, Asp, Arg, Asn, Glu, Gln, Gly, His, Ile, Lys, Leu, Met, Phe, Pro, Ser, Thr, Trp, Tyr, and Val. The conserved cysteine (Cys or C) residue in the wild-type P450 BM3 enzyme is located at position 400 in SEQ ID NO:1. As used herein, the terms “AxX” and “C400X” refer to the presence of an amino acid substitution “X” located at the axial position (i.e., residue 400) of the wild-type P450 BM3 enzyme (i.e., SEQ ID NO:1). In some instances, X is Ser (S). In other instances, X is Ala (A), Asp (D), His (H), Lys (K), Asn (N), Met (M), Thr (T), or Tyr (Y). In some embodiments, the P450 BM3 enzyme variant comprises or consists of the heme domain of the wild-type P450 BM3 enzyme sequence (e.g., amino acids 1-463 of SEQ ID NO: 1) or a fragment thereof and an AxX mutation (i.e., “WT-AxX heme”).

In other embodiments, the P450 BM3 enzyme variant comprises at least one or more (e.g., at least two, three, four, five, six, seven, eight, nine, ten, eleven, twelve, or all thirteen) of the following amino acid substitutions in SEQ ID NO:1: V78A, F87V, P142S, T175I, A184V, S226R, H236Q, E252G, T268A, A290V, L353V, I366V, and E442K. In certain instances, the P450 BM3 enzyme variant comprises a T268A mutation alone or in combination with one or more additional mutations such as a C400X mutation (e.g., C400S) in SEQ ID NO: 1. In other instances, the P450 BM3 enzyme variant comprises all thirteen of these amino acid substitutions (i.e., V78A, F87V, P142S, T175I, A184V, S226R, H236Q, E252G, T268A, A290V, L353V, I366V, and E442K; “BM3-CIS”) in combination with a C400X mutation (e.g., C400S) in SEQ ID NO:1. In some instances, the P450 BM3 enzyme variant comprises or consists of the heme domain of the BM3-CIS enzyme sequence (e.g., amino acids 1-463 of SEQ ID NO: 1 comprising all thirteen of these amino acid substitutions) or a fragment thereof and an “AxX” mutation (i.e., “BM3-CIS-AxX heme”).

In some embodiments, the P450 BM3 enzyme variant further comprises at least one or more (e.g., at least two, or all three) of the following amino acid substitutions in SEQ ID NO:1: I263A, A328G, and a T438 mutation. In certain instances, the T438 mutation is T438A, T438S, or T438P. In some instances, the P450 BM3 enzyme variant comprises a T438 mutation such as T438A, T438S, or T438P alone or in combination with one or more additional mutations such as a C400X mutation (e.g., C400S) in SEQ ID NO:1 or a heme domain or fragment thereof. In other instances, the P450 BM3 enzyme variant comprises a T438 mutation such as T438A, T438S, or T438P in a BM3-CIS backbone alone or in combination with a C400X mutation (e.g., C400S) in SEQ ID NO:1 (i.e., “BM3-CIS-T438S-AxX”). In yet other instances, the P450 BM3 enzyme variant comprises or consists of the heme domain of the BM3-CIS enzyme sequence or a fragment thereof in combination with a T438 mutation and an “AxX” mutation (e.g., “BM3-CIS-T438S-AxX heme”).

In other embodiments, the P450 BM3 enzyme variant further comprises from one to five (e.g., one, two, three, four, or five) active site alanine substitutions in the active site of SEQ ID NO: 1. In certain instances, the active site alanine substitutions are selected from the group consisting of L75A, M177A, L181A, I263A, L437A, and a combination thereof.

In further embodiments, the P450 BM3 enzyme variant comprises at least one or more (e.g., at least about 2, 3, 4, 5, 6, 7, 8, 9, 10, 11, 12, 13, 14, 15, 16, 17, 18, 19, 20, 21, or 22) of the following amino acid substitutions in SEQ ID NO:1: R47C, L52I, I58V, L75R, F81 (e.g., F81L, F81W), A82 (e.g., A82S, A82F, A82G, A82T, etc.), F87A, K94I, I94K, H100R, S106R, F107L, A135S, F162I, A197V, F205C, N239H, R255S, S274T, L324I, A328V, V340M, and K434E. In particular embodiments, the P450 BM3 enzyme variant comprises any one or a plurality of these mutations alone or in combination with one or more additional mutations such as those described above, e.g., an “AxX” mutation and/or at least one or more mutations including V78A, F87V, P142S, T175I, A184V, S226R, H236Q, E252G, T268A, A290V, L353V, I366V, and E442K.

Table 4 below provides non-limiting examples of cytochrome P450 BM3 variants of the present invention. Each P450 BM3 variant comprises one or more of the listed mutations (Variant Nos. 1-31), wherein a “+” indicates the presence of that particular mutation(s) in the variant. Any of the variants listed in Table 4 can further comprise an I263A and/or an A328G mutation and/or at least one, two, three, four, or five of the following alanine substitutions, in any combination, in the P450 BM3 enzyme active site: L75A, M177A, L181A, I263A, and L437A. In particular embodiments, the P450 BM3 variant comprises or consists of the heme domain of any one of Variant Nos. 1-31 listed in Table 4 or a fragment thereof, wherein the fragment is capable of carrying out the cyclopropanation reactions of the present invention.

TABLE 4
Exemplary cytochrome P450 BM3 enzyme variants of the present invention.

| | P450_{BM3} variant | | | | | | | | | | | | | | | |
|---|---|---|---|---|---|---|---|---|---|---|---|---|---|---|---|---|
| Mutation | 1 | 2 | 3 | 4 | 5 | 6 | 7 | 8 | 9 | 10 | 11 | 12 | 13 | 14 | 15 | 16 |
| C400X | + | | | | | + | + | + | + | | | | | | | + |
| T268A | | + | | | | + | | | | + | + | + | | | | + |
| F87V | | | + | | | | + | | | + | | | + | + | | + |
| 9-10A-TS | | | | + | | | | + | | | + | | + | | + | |
| T438Z | | | | | + | | | | + | | | + | | + | + | |

| | P450_{BM3} variant | | | | | | | | | | | | | | |
|---|---|---|---|---|---|---|---|---|---|---|---|---|---|---|---|
| Mutation | 17 | 18 | 19 | 20 | 21 | 22 | 23 | 24 | 25 | 26 | 27 | 28 | 29 | 30 | 31 |
| C400X | + | + | + | + | + | | | | | + | + | + | + | | + |
| T268A | + | + | | | | + | + | + | | + | + | + | | + | + |
| F87V | | | + | + | | + | + | | + | + | + | | + | + | + |
| 9-10A-TS | + | | + | | + | + | | + | + | + | | + | + | + | + |
| T438Z | | + | | + | + | | + | + | + | | + | + | + | + | + |

Mutations relative to the wild-type P450_{BM3} amino acid sequence (SEQ ID NO: 1); “X” is selected from Ala, Asp, Arg, Asn, Glu, Gln, Gly, His, Ile, Lys, Leu, Met, Phe, Pro, Ser, Thr, Trp, Tyr, and Val; “Z” is selected from Ala, Ser, and Pro; “9-10A-TS” includes the following amino acid substitutions in SEQ ID NO: 1: V78A, P142S, T175I, A184V, S226R, H236Q, E252G, A290V, L353V, I366V, and E442K.

One skilled in the art will understand that any of the mutations listed in Table 4 can be introduced into any cytochrome P450 enzyme of interest by locating the segment of the DNA sequence in the corresponding cytochrome P450 gene which encodes the conserved amino acid residue as described above for identifying the conserved cysteine residue in a cytochrome P450 enzyme of interest that serves as the heme axial ligand. In certain instances, this DNA segment is identified through detailed mutagenesis studies in a conserved region of the protein (see, e.g., Shimizu et al., Biochemistry 27, 4138-4141, 1988). In other instances, the conserved amino acid residue is identified through crystallographic study (see, e.g., Poulos et al., J. Mol. Biol 195:687-700, 1987). In yet other instances, protein sequence alignment algorithms can be used to identify the conserved amino acid residue. As non-limiting examples, BLAST alignment can be used with the P450 BM3 amino acid sequence as the query sequence to identify the heme axial ligand site and/or the equivalent T268 residue in other cytochrome P450 enzymes.

Table 5A below provides non-limiting examples of preferred cytochrome P450 BM3 variants of the present invention. Table 5B below provides non-limiting examples of preferred chimeric cytochrome P450 enzymes of the present invention.

TABLE 5A
Exemplary cytochrome P450 BM3 enzyme variants for use in the invention.

| P450_{BM3} variants | Mutations compared to wild-type P450_{BM3} (SEQ ID NO: 1) |
|---|---|
| P450_{BM3}-T268A | T268A |
| P450_{BM3}-T268A-C400H | T268A + C400H |
| P411_{BM3} (ABC) | C400S |
| P411_{BM3}-T268A | T268A + C400S |
| P450_{BM3}-T268A-F87V | T268A + F87V |
| 9-10A TS | V78A, P142S, T175I, A184V, S226R, H236Q, E252G, A290V, L353V, |
| | I366V, E442K |
| 9-10A-TS-F87V | 9-10A TS + F87V |
| H2A10 | 9-10A TS + F87V, L75A, L181A, T268A |
| H2A10-C400S | 9-10A TS + F87V, L75A, L181A, T268A, C400S |
| H2A10-C400H | 9-10A TS + F87V, L75A, L181A, T268A, C400H |
| H2A10-C400M | 9-10A TS + F87V, L75A, L181A, T268A, C400M |
| H2-5-F10 | 9-10A TS + F87V, L75A, I263A, T268A, L437A |
| H2-5-F10-C400S | 9-10A TS + F87V, L75A, I263A, T268A, L437A, C400S |
| H2-5-F10-C400H | 9-10A TS + F87V, L75A, I263A, T268A, L437A, C400H |
| H2-5-F10-C400M | 9-10A TS + F87V, L75A, I263A, T268A, L437A, C400M |
| H2-4-D4 | 9-10A TS + F87V, L75A, M177A, L181A, T268A, L437A |
| H2-4-D4-C400S | 9-10A TS + F87V, L75A, M177A, L181A, T268A, L437A, C400S |
| H2-4-D4-C400H | 9-10A TS + F87V, L75A, M177A, L181A, T268A, L437A, C400H |
| H2-2-A1 | 9-10A TS + F87A, L75A, L181A, L437A |
| H2-2-A1-C400S | 9-10A TS + F87A, L75A, L181A, L437A, C400S |
| H2-2-A1-C400H | 9-10A TS + F87A, L75A, L181A, L437A, C400H |
| H2-8-C7 | 9-10A TS + L75A, F87V, L181A |
| H2-5-F10-A75L | 9-10A TS + F87V-I263A-T268A-L437A |
| CH F8 | R47C, V78A, K94I, P142S, T175I, A184V, F205C, S226R, H236Q, |
| | E252G, R255S, A290V, L353V, F81W, A82S, F87A, A197V |
| BM3-CIS (P450_{BM3}-CIS; C3C) | 9-10A TS + F87V, T268A |
| BM3-CIS-I263A | BM3-CIS + I263A |
| BM3-CIS-A328G | BM3-CIS + A328G |
| BM3-CIS-T438S | BM3-CIS + T438S |
| BM3-CIS-C400S (P411_{BM3}-CIS; | BM3-CIS + C400S |
| ABC-CIS) | |
| BM3-CIS-C400D (BM3-CIS-AxD) | BM3-CIS + C400D |
| BM3-CIS-C400Y (BM3-CIS-AxY) | BM3-CIS + C400Y |
| BM3-CIS-C400K (BM3-CIS-AxK) | BM3-CIS + C400K |
| BM3-CIS-C400H (BM3-CIS-AxH) | BM3-CIS + C400H |
| BM3-CIS-C400M (BM3-CIS-AxM) | BM3-CIS + C400M |
| WT-AxA (heme) | WT heme domain (amino acids 1-463 of SEQ ID NO: 1) + C400A |
| WT-AxD (heme) | WT heme domain (amino acids 1-463 of SEQ ID NO: 1) + C400D |
| WT-AxH (heme) | WT heme domain (amino acids 1-463 of SEQ ID NO: 1) + C400H |
| WT-AxK (heme) | WT heme domain (amino acids 1-463 of SEQ ID NO: 1) + C400K |
| WT-AxM (heme) | WT heme domain (amino acids 1-463 of SEQ ID NO: 1) + C400M |
| WT-AxN (heme) | WT heme domain (amino acids 1-463 of SEQ ID NO: 1) + C400N |
| WT-AxY (heme) | WT heme domain (amino acids 1-463 of SEQ ID NO: 1) + C400Y |
| BM3-CIS-T438S-AxA | BM3-CIS-T438S + C400A |
| BM3-CIS-T438S-AxD | BM3-CIS-T438S + C400D |
| BM3-CIS-T438S-AxM | BM3-CIS-T438S + C400M |
| BM3-CIS-T438S-AxY | BM3-CIS-T438S + C400Y |
| BM3-CIS-T438S-AxT | BM3-CIS-T438S + C400T |
| 7-11D | R47C, V78A, K94I, P142S, T175I, A184V, F205C, S226R, H236Q, |
| | E252G, R255S, A290V, L353V, A82F, A328V |
| 7-11D-C400S | R47C, V78A, K94I, P142S, T175I, A184V, F205C, S226R, H236Q, |
| | E252G, R255S, A290V, L353V, A82F, A328V, C400S |
| 12-10C | R47C, V78A, A82G, F87V, K94I, P142S, T175I, A184V, F205C, |
| | S226R, H236Q, E252G, R255S, A290V, A328V, L353V |
| 22A3 | L52I, I58V, L75R, F87A, H100R, S106R, F107L, A135S, F162I, |
| | A184V, N239H, S274T, L324I, V340M, I366V, K434E |
| Man1 | V78A, K94I, P142S, T175I, A184V, F205C, S226R, H236Q, E252G, |
| | R255S, A290V, L353V, F81L, A82T, F87A, I94K |

TABLE 5B
Exemplary chimeric cytochrome P450 enzymes for use the invention.

| Chimeric P450s | Heme domain block sequence | SEQ ID NO |
|---|---|---|
| C2G9 | 22223132 | 43 |
| X7 | 22312333 | 44 |
| X7-12 | 12112333 | 45 |
| C2E6 | 11113311 | 46 |
| X7-9 | 32312333 | 47 |
| C2B12 | 32313233 | 48 |
| TSP234 | 22313333 | 49 |

In particular embodiments, cytochrome P450 BM3 variants with at least one or more amino acid mutations such as, e.g., C400X (AxX), BM3-CIS, T438, and/or T268A amino acid substitutions catalyze cyclopropanation reactions efficiently, displaying increased total turnover numbers and demonstrating highly regio- and/or enantioselective product formation compared to the wild-type enzyme.

As a non-limiting example, certain cytochrome P450 BM3 variants of the present invention are cis-selective catalysts that demonstrate diastereomeric ratios at least comparable to wild-type P450 BM3, e.g., at least 37:63 cis:trans, at least 50:50 cis:trans, at least 60:40 cis:trans, or at least 95:5 cis:trans. Particular mutations for improving cis-selective catalysis include at least one mutation comprising T268A, C400X, and T438S, but preferably one, two, or all three of these mutations in combination with additional mutations comprising V78A, P142S, T175I, A184V, S226R, H236Q, E252G, A290V, L353V, I366V, E442K, and F87V derived from P450 BM3 variant 9-10A-TS. These mutations are isolated to the heme domain of P450 BM3 and are located in various regions of the heme domain structure including the active site and periphery.

As another non-limiting example, certain cytochrome P450 BM3 variants of the present invention are trans-selective catalysts that demonstrate diastereomeric ratios at least comparable to wild-type P450 BM3, e.g., at least 37:63 cis:trans, at least 20:80 cis:trans, or at least 1:99 cis:trans. Particular mutations for improving trans-selective catalysis include at least one mutation comprising including T268A and C400X, but preferably one or both of these mutations in the background of wild-type P450 BM3. In certain embodiments, trans-preferential mutations in combination with additional mutations such as V78A, P142S, T175I, A184V, S226R, H236Q, E252G, A290V, L353V, I366V, E442K, and F87V (from 9-10-A-TS) are also tolerated when in the presence of additional mutations including, but not limited to, I263A, L437A, L181A and/or L75A. These mutations are isolated to the heme domain of P450 BM3 and are located in various regions of the heme domain structure including the active site and periphery.

In certain embodiments, the present invention also provides P450 variants that catalyze enantioselective cyclopropanation with enantiomeric excess values of at least 30% (comparable with wild-type P450 BM3), but more preferably at least 80%, and even more preferably at least >95% for preferred product diastereomers.

In other aspects, the present invention provides chimeric heme enzymes such as, e.g., chimeric P450 proteins comprised of recombined sequences from P450 BM3 and at least one, two, or more distantly related P450 enzymes from Bacillus subtilis or any other organism that are competent cyclopropanation catalysts using similar conditions to wild-type P450 BM3 and highly active P450 BM3 variants. As a non-limiting example, site-directed recombination of three bacterial cytochrome P450s can be performed with sequence crossover sites selected to minimize the number of disrupted contacts within the protein structure. In some embodiments, seven crossover sites can be chosen, resulting in eight sequence blocks. One skilled in the art will understand that the number of crossover sites can be chosen to produce the desired number of sequence blocks, e.g., 1, 2, 3, 4, 5, 6, 7, 8, or 9 crossover sites for 2, 3, 4, 5, 6, 7, 8, 9, or 10 sequence blocks, respectively. In other embodiments, the numbering used for the chimeric P450 refers to the identity of the parent sequence at each block. For example, “12312312” refers to a sequence containing block 1 from P450 #1, block 2 from P450 #2, block 3 from P450 #3, block 4 from P450 #1, block 5 from P450 #2, and so on. A chimeric library useful for generating the chimeric heme enzymes of the invention can be constructed as described in, e.g., Otey et al., PLoS Biology, 4(5):e112 (2006), following the SISDC method (see, Hiraga et al., J. Mol. Biol., 330:287-96 (2003)) using the type IIb restriction endonuclease BsaXI, ligating the full-length library into the pCWori vector and transforming into the catalase-deficient E. coli strain SN0037 (see, Nakagawa et al., Biosci. Biotechnol. Biochem., 60:415-420 (1996)); the disclosures of these references are hereby incorporated by reference in their entirety for all purposes.

As a non-limiting example, chimeric P450 proteins comprising recombined sequences or blocks of amino acids from CYP102A1 (Accession No. J04832), CYP102A2 (Accession No. CAB12544), and CYP102A3 (Accession No. U93874) can be constructed. In certain instances, the CYP102A1 parent sequence is assigned “1”, the CYP102A2 parent sequence is assigned “2”, and the CYP102A3 is parent sequence assigned “3”. In some instances, each parent sequence is divided into eight sequence blocks containing the following amino acids (aa): block 1: aa 1-64; block 2: aa 65-122; block 3: aa 123-166; block 4: aa 167-216; block 5: aa 217-268; block 6: aa 269-328; block 7: aa 329-404; and block 8: aa 405-end. Thus, in this example, there are eight blocks of amino acids and three fragments are possible at each block. For instance, “12312312” refers to a chimeric P450 protein of the invention containing block 1 (aa 1-64) from CYP102A1, block 2 (aa 65-122) from CYP102A2, block 3 (aa 123-166) from CYP102A3, block 4 (aa 167-216) from CYP102A1, block 5 (aa 217-268) from CYP102A2, and so on. See, e.g., Otey et al., PLoS Biology, 4(5):e112 (2006). Non-limiting examples of chimeric P450 proteins include those set forth in Table 5B (C₂G9, X7, X7-12, C2E6, X7-9, C2B12, TSP234). In some embodiments, the chimeric heme enzymes of the invention can comprise at least one or more of the mutations described herein.

In some embodiments, the present invention provides the incorporation of homologous or analogous mutations to C400X (AxX) and/or T268A in other cytochrome P450 enzymes and heme enzymes in order to impart or enhance cyclopropanation activity.

As non-limiting examples, the cytochrome P450 can be a variant of CYP101A1 (SEQ ID NO:25) comprising a C357X (e.g., C357S) mutation, a T252A mutation, or a combination of C357X (e.g., C357S) and T252A mutations, wherein “X” is any amino acid other than Cys, or the cytochrome P450 can be a variant of CYP2B4 (SEQ ID NO:28) comprising a C436X (e.g., C436S) mutation, a T302A mutation, or a combination of C436X (e.g., C436S) and T302A mutations, wherein “X” is any amino acid other than Cys, or the cytochrome P450 can be a variant of CYP2D7 (SEQ ID NO:26) comprising a C461X (e.g., C461 S) mutation, wherein “X” is any amino acid other than Cys, or the cytochrome P450 can be a variant of P450C27 (SEQ ID NO:27) comprising a C478X (e.g., C478S) mutation, wherein “X” is any amino acid other than Cys.

In other embodiments, the heme protein is a cytochrome c or a variant thereof. In a particular embodiment, the heme protein is a mature cytochrome c protein (residues 29-152 of the unprocessed peptide) B3FQS5_RHOMR (Swiss-Prot: B3FQS5) from Rhodothermus marinus (Rhodothermus obamensis) or a variant thereof (Biochemistry 2008, 47, 11953-11963). In a further embodiment, the B3FQS5_RHOMR protein (Rma cyt c) contains a mutation at the axial heme ligand residue M100 (mature peptide numbering convention) to any other amino acid residue that is among the naturally occurring twenty amino acids. In a further embodiment, the Rma cyt c protein consists of a single mutation of the position V75 to any other amino acid. In a further embodiment, the Rma cyt c protein consists of any combination of mutations residues M100 and V75 to any other amino acid.

In some embodiments, the heme protein is a cytochrome c protein or a variant thereof. In a particular embodiment, the heme protein is a cytochrome c protein CYC2_RHOGL (Swiss-Prot: P00080) from Rhodopila globiformis (Rhodopsuedomonas globiformis) or a variant thereof (Arch. Biochem. Biophys. 1996, 333, 338-348). In a particular embodiment, the heme protein is a mature cytochrome c protein (residues 19-98 of the unprocessed peptide) CY552_HYDTT (Swiss-Prot: P15452) from Hydrogenobacter thermophilus (strain DSM 6534/IAM 12695/TK-6) or a variant thereof (J. Biol. Chem. 2005, 280, 25729-25734). In a further embodiment, the CY552_HYDTT protein (Hth cyt c) contains a mutation at the axial heme ligand residue M59 (mature peptide numbering convention) to any other amino acid residue that is among the naturally occurring twenty amino acids. In a further embodiment, the Hth cyt c protein consists of a single mutation of the position Q62 to any other amino acid. In a further embodiment, the Hth cyt c protein consists of any combination of mutations residues M59 and Q62 to any other amino acid.

In certain embodiments, the heme protein is a globin or a variant thereof. In a particular embodiment, the heme protein is a Hell's Gate globin B3DUZ7_METI4 (Swiss-Prot: B3DUZ7) from Methylacidiphilum infernorum (Methylokorus infernorum) or a variant thereof. In a further embodiment, the Hell's Gate globin (HGG) contains a mutation at residue Y29 to any other amino acid residue that is among the naturally occurring twenty amino acids. In a further embodiment, the HGG protein consists of a single mutation of the position Q50 to any other amino acid. In a further embodiment, the HGG protein consists of any combination of mutations residues Y29 and Q50 to any other amino acid.

In some embodiments, the globin is myoglobin or a variant thereof. In some embodiments, the globin is M. infernorum hemoglobin according to SEQ ID NO:61 or a variant thereof. In some embodiments, the M. infernorum hemoglobin variant comprises one or more mutations of amino acid residues selected from the group consisting of F28, Y29, L32, L54, and V95. In some embodiments, the M. infernorum hemoglobin variant comprises one or more mutations selected from the group consisting of F28S, Y29A, L32A, L32C, L32T, L54S, and V95F. In some such embodiments, the M. infernorum variant comprises a V95F mutation.

In some embodiments, the globin is B. subtilis truncated hemoglobin according to SEQ ID NO:62 or a variant thereof. In some embodiments, the B. subtilis hemoglobin variant comprises one or more mutations of amino acid residues selected from the group consisting of T45 and Q49. In some embodiments, the B. subtilis hemoglobin variant comprises a T45 mutation and a Q49 mutation. In some embodiments, the B. subtilis hemoglobin variant comprises one or more mutations selected from the group consisting of T45L, T45F, T45A, Q49L, Q49F, and Q49A. In some such embodiments, the B. subtilis hemoglobin variant comprises a first mutation selected from the group consisting of T45L, T45F, and T45A, and a second mutation selected from the group consisting of Q49L, Q49F, and Q49A.

In some embodiments, the heme protein is a myoglobin or a variant thereof. In a particular embodiment, the heme protein is sperm whale myoglobin or a variant thereof. In a further embodiment, the myoglobin protein (Mb) contains a mutation at residue H64 to any other amino acid residue that is among the naturally occurring twenty amino acids. In a further embodiment, the Mb protein contains a single mutation of the position V68 to any other amino acid. In a further embodiment, the Mb protein contains any combination of mutations of residues M64 and V68 to any other amino acid.

In some embodiments, the heme protein is a peroxidase or a variant thereof. In some embodiments, the heme protein is a catalase or a variant thereof.

An enzyme's total turnover number (or TTN) refers to the maximum number of molecules of a substrate that the enzyme can convert before becoming inactivated. In general, the TTN for the heme enzymes of the invention range from about 1 to about 100,000 or higher. For example, the TTN can be from about 1 to about 1,000, or from about 1,000 to about 10,000, or from about 10,000 to about 100,000, or from about 50,000 to about 100,000, or at least about 100,000. In particular embodiments, the TTN can be from about 100 to about 10,000, or from about 10,000 to about 50,000, or from about 5,000 to about 10,000, or from about 1,000 to about 5,000, or from about 100 to about 1,000, or from about 250 to about 1,000, or from about 100 to about 500, or at least about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, or more. In certain embodiments, the variant or chimeric heme enzymes of the present invention have higher TTNs compared to the wild-type sequences. In some instances, the variant or chimeric heme enzymes have TTNs greater than about 100 (e.g., at least about 100, 150, 200, 250, 300, 325, 350, 400, 450, 500, or more) in carrying out in vitro cyclopropanation reactions. In other instances, the variant or chimeric heme enzymes have TTNs greater than about 1000 (e.g., at least about 1000, 2500, 5000, 10,000, 25,000, 50,000, 75,000, 100,000, or more) in carrying out in vivo whole cell cyclopropanation reactions.

In certain embodiments, the present invention provides heme enzymes such as the P450 variants described herein that are active cyclopropanation catalysts inside living cells. As a non-limiting example, bacterial cells (e.g., E. coli) can be used as whole cell catalysts for the in vivo cyclopropanation reactions of the present invention. In some embodiments, whole cell catalysts containing P450 enzymes with the equivalent C400X mutation are found to significantly enhance the total turnover number (TTN) compared to in vitro reactions using isolated P450 enzymes.

When whole cells expressing a heme enzyme are used to carry out a cyclopropanation reaction, the turnover can be expressed as the amount of substrate that is converted to product by a given amount of cellular material. In general, in vivo cyclopropanation reactions exhibit turnovers from at least about 0.01 to at least about 10 mmol·g_{cdw}⁻¹, wherein g_{cdw} is the mass of cell dry weight in grams. For example, the turnover can be from about 0.1 to about 10 mmol·g_{cdw}⁻¹, or from about 1 to about 10 mmol·g_{cdw}⁻¹, or from about 5 to about 10 mmol·g_{cdw}⁻¹, or from about 0.01 to about 1 mmol·g_{cdw}⁻¹, or from about 0.01 to about 0.1 mmol·g_{cdw}⁻¹, or from about 0.1 to about 1 mmol·g_{cdw}⁻¹, or greater than 1 mmol·g_{cdw}⁻¹. The turnover can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 mmol·g_{cdw}⁻¹.

When whole cells expressing a heme enzyme are used to carry out a cyclopropanation reaction, the activity can further be expressed as a specific productivity, e.g., concentration of product formed by a given concentration of cellular material per unit time, e.g., in g/L of product per g/L of cellular material per hour (g g_{cdw}⁻¹ h⁻¹). In general, in vivo cyclopropanation reactions exhibit specific productivities from at least about 0.01 to at least about 0.5 g·g_{cdw}⁻¹ h⁻¹, wherein g_{cdw} is the mass of cell dry weight in grams. For example, the specific productivity can be from about 0.01 to about 0.1 g g_{cdw}⁻¹ h⁻¹, or from about 0.1 to about 0.5 g g_{cdw}⁻¹ h⁻¹, or greater than 0.5 g g_{cdw}⁻¹ h⁻¹. The specific productivity can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, or about 0.5 g g_{cdw}⁻¹ h⁻¹.

In certain embodiments, mutations can be introduced into the target gene using standard cloning techniques (e.g., site-directed mutagenesis) or by gene synthesis to produce the heme enzymes (e.g., cytochrome P450 variants) of the present invention. The mutated gene can be expressed in a host cell (e.g., bacterial cell) using an expression vector under the control of an inducible promoter or by means of chromosomal integration under the control of a constitutive promoter. Cyclopropanation activity can be screened in vivo or in vitro by following product formation by GC or HPLC as described herein.

The expression vector comprising a nucleic acid sequence that encodes a heme enzyme of the invention can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). Expression vectors can include chromosomal, non-chromosomal, and synthetic DNA sequences. Equivalent expression vectors to those described herein are known in the art and will be apparent to the ordinarily skilled artisan.

The expression vector can include a nucleic acid sequence encoding a heme enzyme that is operably linked to a promoter, wherein the promoter comprises a viral, bacterial, archaeal, fungal, insect, or mammalian promoter. In certain embodiments, the promoter is a constitutive promoter. In some embodiments, the promoter is an inducible promoter. In other embodiments, the promoter is a tissue-specific promoter or an environmentally regulated or a developmentally regulated promoter.

It is understood that affinity tags may be added to the N- and/or C-terminus of a heme enzyme expressed using an expression vector to facilitate protein purification. Non-limiting examples of affinity tags include metal binding tags such as His6-tags and other tags such as glutathione S-transferase (GST).

Non-limiting expression vectors for use in bacterial host cells include pCWori, pET vectors such as pET22 (EMD Millipore), pBR322 (ATCC37017), pQE™ vectors (Qiagen), pBluescript™ vectors (Stratagene), pNH vectors, lambda-ZAP vectors (Stratagene); ptrc99a, pKK223-3, pDR540, pRIT2T (Pharmacia), pRSET, pCR-TOPO vectors, pET vectors, pSyn_1 vectors, pChlamy_1 vectors (Life Technologies, Carlsbad, Calif.), pGEM1 (Promega, Madison, Wis.), and pMAL (New England Biolabs, Ipswich, Mass.). Non-limiting examples of expression vectors for use in eukaryotic host cells include pXT1, pSG5 (Stratagene), pSVK3, pBPV, pMSG, pSVLSV40 (Pharmacia), pcDNA3.3, pcDNA4/TO, pcDNA6/TR, pLenti6/TR, pMT vectors (Life Technologies), pKLAC1 vectors, pKLAC2 vectors (New England Biolabs), pQE™ vectors (Qiagen), BacPak baculoviral vectors, pAdeno-X™ adenoviral vectors (Clontech), and pBABE retroviral vectors. Any other vector may be used as long as it is replicable and viable in the host cell.

The host cell can be a bacterial cell, an archaeal cell, a fungal cell, a yeast cell, an insect cell, or a mammalian cell.

Suitable bacterial host cells include, but are not limited to, BL21 E. coli, DE3 strain E. coli, E. coli M15, DH5α, DH10β, HB101, T7 Express Competent E. coli (NEB), B. subtilis cells, Pseudomonas fluorescens cells, and cyanobacterial cells such as Chlamydomonas reinhardtii cells and Synechococcus elongates cells. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. Fungal host cells include, but are not limited to, yeast cells from the genera Saccharomyces (e.g., S. cerevisiae), Pichia (P. Pastoris), Kluyveromyces (e.g., K. lactis), Hansenula and Yarrowia, and filamentous fungal cells from the genera Aspergillus, Trichoderma, and Myceliophthora. Suitable insect host cells include, but are not limited to, Sf9 cells from Spodoptera frugiperda, Sf21 cells from Spodoptera frugiperda, Hi-Five cells, BTI-TN-5B1-4 Trichophusia ni cells, and Schneider 2 (S2) cells and Schneider 3 (S3) cells from Drosophila melanogaster. Non-limiting examples of mammalian host cells include HEK293 cells, HeLa cells, CHO cells, COS cells, Jurkat cells, NSO hybridoma cells, baby hamster kidney (BHK) cells, MDCK cells, NIH-3T3 fibroblast cells, and any other immortalized cell line derived from a mammalian cell.

In another aspect, the invention provides methods for preparing cyclopropanation products using M. infernorum hemoglobin or B. subtilis hemoglobin, and variants thereof, as catalysts. In some embodiments, the method includes: (al) providing an olefinic substrate, a diazo reagent, and M. infernorum hemoglobin, or a variant thereof; and (b1) admixing the components of step (al) in a reaction for a time sufficient to produce a cyclopropanation product. In some embodiments, the method includes: (a2) providing an olefinic substrate, a diazo reagent, and B. subtilis hemoglobin, or a variant thereof; and (b2) admixing the components of step (a2) in a reaction for a time sufficient to produce a cyclopropanation product.

In some embodiments, the cyclopropanation product is a compound according to Formula L:

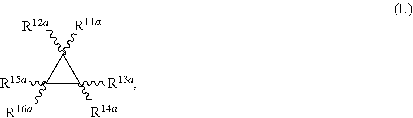

wherein:
R^{11a} is independently selected from the group consisting of H, optionally substituted C₁₋₁₈ alkyl, optionally substituted C₆₋₁₀ aryl, optionally substituted 6- to 10-membered heteroaryl, halo, cyano, C(O)OR^{11b}, C(O)N(R^{17a})₂, C(O)R^{18a}, C(O)C(O)OR^{18a}, and Si(R^{18a})₃;
R^{12a} is independently selected from the group consisting of H, optionally substituted C₁₋₁₈ alkyl, optionally substituted C₆₋₁₀ aryl, optionally substituted 6- to 10-membered heteroaryl, halo, cyano, C(O)OR^{12b}, C(O)N(R¹⁷)₂, C(O)R^{18a}, C(O)C(O)OR^{18a}, and Si(R^{18a})₃;
wherein:
   R^{11b} and R^{12b} are independently selected from the group consisting of H, optionally substituted C₁₋₁₈ alkyl and -L-R^{C}, wherein
   each L is selected from the group consisting of a bond, —C(R^{L})₂—, and —NR^{L}—C(R^{L})₂—,
   each R^{L} is independently selected from the group consisting of H, C₁₋₆ alkyl, halo, —CN, and —SO₂, and
   each R^{C} is selected from the group consisting of optionally substituted C₆₋₁₀ aryl, optionally substituted 6- to 10-membered heteroraryl, and optionally substituted 6- to 10-membered heterocyclyl; and
R^{13a}, R^{14a}, R^{15a}, and R^{16a} are independently selected from the group consisting of H, C₁₋₁₈ alkyl, C₂₋₁₈ alkenyl, C₂₋₁₈ alkynyl, optionally substituted C₆₋₁₀ aryl, optionally substituted C₁-C₆ alkoxy, halo, hydroxy, cyano, C(O)N(R^{17a})₂, NR^{17a}C(O)R^{18a}, C(O)R^{18a}, C(O)OR^{18a}, and N(R^{19a})₂,
wherein:
   each R^{17a} and R^{18a} is independently selected from the group consisting of H, optionally substituted C₁₋₁₂ alkyl, optionally substituted C₂₋₁₂ alkenyl, and optionally substituted C₆₋₁₀ aryl; and
   each R^{19a} is independently selected from the group consisting of H, optionally substituted C₆₋₁₀ aryl, and optionally substituted 6- to 10-membered heteroaryl, or two R^{19a} moieties, together with the nitrogen atom to which they are attached, can form 6- to 18-membered heterocyclyl;
or R^{13a} forms an optionally substituted 3- to 18-membered ring with R⁴;
or R^{15a} forms an optionally substituted 3- to 18-membered ring with R⁶;
or R^{13a} or R^{14a} forms a double bond with R^{15a} or R16a;
or R^{13a} or R^{14a} forms an optionally substituted 5- to 6-membered ring with R^{15a} or R^{16a}.

M. infernorum hemoglobin and B. subtilis hemoglobin, or variants thereof, can be used for preparing a number of cyclopropanation products including, but not limited to, commodity and fine chemicals, flavors and scents, insecticides, and active ingredients in pharmaceutical compositions. The cyclopropanation products can also serve as starting materials or intermediates for the synthesis of compounds belonging to these and other classes. For example, M. infernorum hemoglobin, B. subtilis hemoglobin, and variants thereof can be used in the methods of the invention for preparation of pyrethroids, milnacipran, bicifidine, cilastain, boceprevir, sitafloxacin, sitafloxacin, anthoplalone, noranthoplone, odanacatib, montekulast, montekulast. These and other cyclopropanation products are described, for example, in U.S. Pat. No. 8,993,262, which is incorporated herein by reference in its entirety.

B. Cyclopropanation Substrates

In some embodiments, the invention provides a method for producing a cyclopropanation product of Formula A:

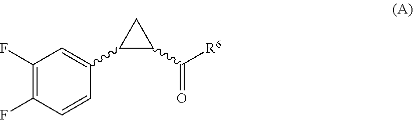

wherein R⁶ is C₁₋₁₈ alkoxy. The method includes combining an olefinic substrate, a diazoester carbene precursor, and a heme enzyme under conditions sufficient to form the product of Formula A.

In some embodiments, the cyclopropanation product is a compound of Formula XVII:

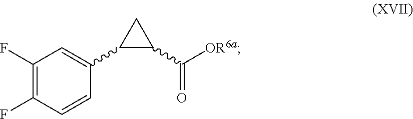

the olefinic substrate is a compound of Formula V

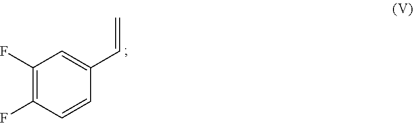

and
the carbene precursor is a compound of Formula XVI,

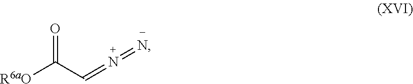

wherein R^{6a} is C₁₋₁₈ alkyl.

In some embodiments, the cyclopropanation product is a compound according to Formula XVIIa:

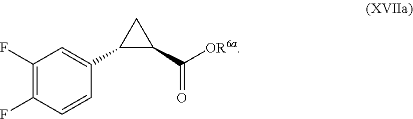

In some embodiments, R^{6a} is selected from the group consisting of C₁₋₈ alkyl, C₁₋₁₂ alkyl, C₁₋₆ alkyl, and C₁₋₄ alkyl. In some embodiments, R^{6a} is selected from the group consisting of methyl, ethyl, propyl, isopropyl, butyl, isobutyl, sec-butyl, tert-butyl, pentyl, isopentyl, and hexyl. In some embodiments, R^{6a} is ethyl. In some embodiments, the cyclopropanation product is a compound according to Formula VIIa:

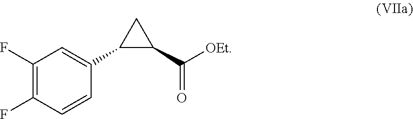 [SMILES: CCOC(=O)[C@@H]1C[C@H]1c1ccc(F)c(F)c1]

In particular embodiments, the present invention provides a method for the synthesis of trans-(1R,2S)-2-(3,4-difluorophenyl)-cyclopropylamine catalyzed by a heme enzyme such as a cytochrome P450 enzyme (e.g., P450 BM3 enzyme) using a diazoketone as the carbene precursor and the Beckmann rearrangement as shown in Scheme 5 above, wherein R is an optionally substituted C₁₋₁₈ alkyl, alkenyl, or alkynyl, or an optionally substituted C₆₋₁₀ aryl or heteroaryl.

In certain aspects, the methods of the present invention for the enzymatic synthesis of trans-(1R,2S)-2-(3,4-difluorophenyl)-cyclopropylamine comprises incubating an olefinic substrate such as a styrene and a carbene precursor such as a diazoketone reagent with a cyclopropanation catalyst such as a heme enzyme to form a cyclopropane product.

In some embodiments, the styrene has a structure according to Formula XXX:

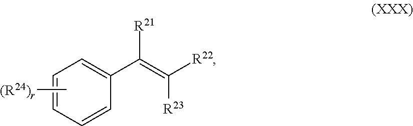

wherein R²¹ is selected from H, optionally substituted C₁-C₆ alkyl, optionally substituted C₁-C₆ alkoxy, C(O)N(R²⁷)₂, C(O)OR²⁸, N(R²⁹)₂, halo, hydroxy, and cyano. R²² and R²³ are independently selected from H, optionally substituted C₁₋₆ alkyl, and halo. R²⁴ is selected from optionally substituted C₁-C₆ alkyl, optionally substituted C₁-C₆ alkoxy, halo, and haloalkyl, and the subscript r is an integer from 0 to 2.

In particular embodiments, R²¹, R²² and R²³ are all H, R²⁴ is a halogen such as fluorine, and r is 2. In preferred embodiments, the styrene is 1,2-difluoro-4-vinylbenzene (i.e., 3,4-difluorostyrene).

In some embodiments, the diazoketone a structure according to Formula XXXI:

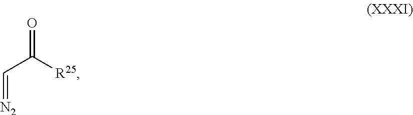

wherein R²⁵ is selected from an optionally substituted C₁₋₁₈ alkyl, alkenyl, or alkynyl, or an optionally substituted C₆₋₁₀ aryl or heteroaryl.

Accordingly, some embodiments of the invention provide a method for producing a cyclopropanation product of Formula A:

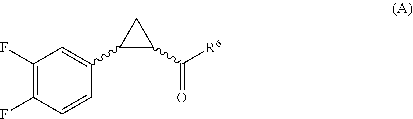

wherein R⁶ is selected from the group consisting of C₁₋₁₈ alkyl, C₁₋₁₈ alkenyl, and C₁₋₁₈ alkynyl. The method includes combining an olefinic substrate, a diazoketone carbene precursor, and a heme enzyme under conditions sufficient to form the product of Formula A.

In some embodiments, the cyclopropanation product is a compound of Formula XXVII:

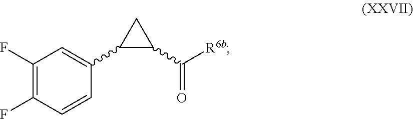

the olefinic substrate is a compound of Formula V:

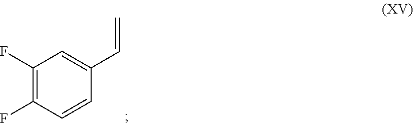

and
the carbene precursor is a compound of Formula XXVI:

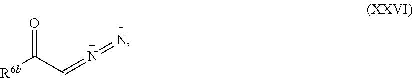

wherein R^{6b} is C₁₋₁₈ alkyl.

In some embodiments, the cyclopropanation product is a compound according to Formula XXVIIa:

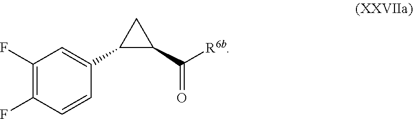

In some embodiments, R^{6b} is selected from the group consisting of C₁₋₈ alkyl, C₁₋₁₂ alkyl, C₁₋₆ alkyl, and C₁₋₄ alkyl. In some embodiments, R^{6b} is selected from the group consisting of methyl, ethyl, propyl, isopropyl, butyl, isobutyl, sec-butyl, tert-butyl, pentyl, isopentyl, and hexyl. In some embodiments, R^{6b} is methyl.

One of skill in the art will appreciate that the stereochemical configuration of the cyclopropanation product will be determined in part by the orientation of the carbene precursor reagent (i.e., the diazoester or the diazoketone) with respect to the position of an olefinic substrate such as styrene during the cyclopropanation step. For example, any substituent originating from the olefinic substrate can be positioned on the same side of the cyclopropyl ring as a substituent originating from the carbene precursor reagent. Cyclopropanation products having this arrangement are called “cis” compounds or “Z” compounds. Any substituent originating from the olefinic substrate and any substituent originating from the carbene precursor reagent can also be on opposite sides of the cyclopropyl ring. Cyclopropanation products having this arrangement are called “trans” compounds or “E” compounds.

Two cis isomers and two trans isomers can arise from the reaction of an olefinic substrate with a carbene precursor reagent. The two cis isomers are enantiomers with respect to one another, in that the structures are non-superimposable mirror images of each other. Similarly, the two trans isomers are enantiomers. One of skill in the art will appreciate that the absolute stereochemistry of a cyclopropanation product—that is, whether a given chiral center exhibits the right-handed “R” configuration or the left-handed “S” configuration—will depend on factors including the structures of the particular olefinic substrate and carbene precursor reagent used in the reaction, as well as the identity of the enzyme. This is also true for the relative stereochemistry—that is, whether a cyclopropanation product exhibits a cis or trans configuration—as well as for the distribution of cyclopropanation product mixtures will also depend on such factors.

In general, cyclopropanation product mixtures have cis:trans ratios ranging from about 1:99 to about 99:1. The cis:trans ratio can be, for example, from about 1:99 to about 1:75, or from about 1:75 to about 1:50, or from about 1:50 to about 1:25, or from about 99:1 to about 75:1, or from about 75:1 to about 50:1, or from about 50:1 to about 25:1. The cis:trans ratio can be from about 1:80 to about 1:20, or from about 1:60 to about 1:40, or from about 80:1 to about 20:1 or from about 60:1 to about 40:1. The cis:trans ratio can be about 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, or about 1:95. The cis:trans ratio can be about 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, or about 95:1.

The distribution of a cyclopropanation product mixture can be assessed in terms of the enantiomeric excess, or “% ee,” of the mixture. The enantiomeric excess refers to the difference in the mole fractions of two enantiomers in a mixture. The enantiomeric excess of the “E” or trans (R,R) and (S,S) enantiomers, for example, can be calculated using the formula: % ee_{E}=[(χ_{R,R}−χ_{S,S})/(χ_{R,R}+χ_{S,S})]×100%, wherein χ is the mole fraction for a given enantiomer. The enantiomeric excess of the “Z” or cis enantiomers (% ee_{Z}) can be calculated in the same manner.

In general, cyclopropanation product mixtures exhibit % ee values ranging from about 1% to about 99%, or from about −1% to about −99%. The closer a given % ee value is to 99% (or −99%), the purer the reaction mixture is. The % ee can be, for example, from about −90% to about 90%, or from about −80% to about 80%, or from about −70% to about 70%, or from about −60% to about 60%, or from about −40% to about 40%, or from about −20% to about 20%. The % ee can be from about 1% to about 99%, or from about 20% to about 80%, or from about 40% to about 60%, or from about 1% to about 25%, or from about 25% to about 50%, or from about 50% to about 75%. The % ee can be from about −1% to about −99%, or from about −20% to about −80%, or from about −40% to about −60%, or from about −1% to about −25%, or from about −25% to about −50%, or from about −50% to about −75%. The % ee can be about −99%, −95%, −90%, −85%, −80%, −75%, −70%, −65%, −60%, −55%, −50%, −45%, −40%, −35%, −30%, −25%, −20%, −15%, −10%, 10%, 15%, 20%, 25%, 30%, 35%, 40%, 45%, 50%, 55%, 60%, 65%, 70%, 75%, 80%, 85%, 90%, or about 95%. Any of these values can be % ee_{E} values or % ee_{Z} values.

Accordingly, some embodiments of the invention provide methods for producing a plurality of cyclopropanation products having a % ee_{Z} of from about −90% to about 90%. In some embodiments, the % ee_{Z} is at least 90%. In some embodiments, the % ee_{Z} is at least −99%. In some embodiments, the % ee_{E} is from about −90% to about 90%. In some embodiments, the % ee_{E} is at least 90%. In some embodiments, the % ee_{E} is at least −99%.

C. Reaction Conditions

The methods of the invention include forming reaction mixtures that contain the heme enzymes described herein. The heme enzymes can be, for example, purified prior to addition to a reaction mixture or secreted by a cell present in the reaction mixture. The reaction mixture can contain a cell lysate including the enzyme, as well as other proteins and other cellular materials. Alternatively, a heme enzyme can catalyze the reaction within a cell expressing the heme enzyme. Any suitable amount of heme enzyme can be used in the methods of the invention. In general, cyclopropanation reaction mixtures contain from about 0.01 mol % to about 10 mol % heme enzyme with respect to the diazo reagent (e.g., diazoketone) and/or olefinic substrate. The reaction mixtures can contain, for example, from about 0.01 mol % to about 0.1 mol % heme enzyme, or from about 0.1 mol % to about 1 mol % heme enzyme, or from about 1 mol % to about 10 mol % heme enzyme. The reaction mixtures can contain from about 0.05 mol % to about 5 mol % heme enzyme, or from about 0.05 mol % to about 0.5 mol % heme enzyme. The reaction mixtures can contain about 0.1, 0.2, 0.3, 0.4, 0.5, 0.6, 0.7, 0.8, 0.9, or about 1 mol % heme enzyme.

The concentration of olefinic substrate and carbene precursor reagent are typically in the range of from about 100 μM to about 1 M. The concentration can be, for example, from about 100 μM to about 1 mM, or about from 1 mM to about 100 mM, or from about 100 mM to about 500 mM, or from about 500 mM to 1 M. The concentration can be from about 500 μM to about 500 mM, 500 μM to about 50 mM, or from about 1 mM to about 50 mM, or from about 15 mM to about 45 mM, or from about 15 mM to about 30 mM. The concentration of olefinic substrate or carbene precursor reagent can be, for example, about 100, 200, 300, 400, 500, 600, 700, 800, or 900 μM. The concentration of olefinic substrate or carbene precursor reagent can be about 1, 5, 10, 15, 20, 25, 30, 35, 40, 45, 50, 55, 60, 65, 70, 75, 80, 85, 90, 95, 100, 150, 200, 250, 300, 350, 400, 450, or 500 mM.

Cyclopropanation reaction mixtures can contain additional reagents. As non-limiting examples, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, isopropanol, glycerol, tetrahydrofuran, acetone, acetonitrile, and acetic acid), salts (e.g., NaCl, KCl, CaCl₂, and salts of Mn²⁺ and Mg²⁺), denaturants (e.g., urea and guanidinium hydrochloride), detergents (e.g., sodium dodecylsulfate and Triton-X 100), chelators (e.g., ethylene glycol-bis(2-aminoethylether)-N,N,N′,N′-tetraacetic acid (EGTA), 2-({2-[Bis(carboxymethyl)amino]ethyl}(carboxymethyl)amino)acetic acid (EDTA), and 1,2-bis(o-aminophenoxy)ethane-N,N,N′,N′-tetraacetic acid (BAPTA)), sugars (e.g., glucose, sucrose, and the like), and reducing agents (e.g., sodium dithionite, NADPH, dithiothreitol (DTT), β-mercaptoethanol (BME), and tris(2-carboxyethyl)phosphine (TCEP)). Buffers, cosolvents, salts, denaturants, detergents, chelators, sugars, and reducing agents can be used at any suitable concentration, which can be readily determined by one of skill in the art. In general, buffers, cosolvents, salts, denaturants, detergents, chelators, sugars, and reducing agents, if present, are included in reaction mixtures at concentrations ranging from about 1 μM to about 1 M. For example, a buffer, a cosolvent, a salt, a denaturant, a detergent, a chelator, a sugar, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, a reducing agent is used in a sub-stoichiometric amount with respect to the olefin substrate and the carbene precursor reagent. Cosolvents, in particular, can be included in the reaction mixtures in amounts ranging from about 1% v/v to about 75% v/v, or higher. A cosolvent can be included in the reaction mixture, for example, in an amount of about 5, 10, 20, 30, 40, or 50% (v/v).

Reactions are conducted under conditions sufficient to catalyze the formation of a cyclopropanation product. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 6 to about 10. The reactions can be conducted, for example, at a pH of from about 6.5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Reactions can be conducted under aerobic conditions or anaerobic conditions. Reactions can be conducted under an inert atmosphere, such as a nitrogen atmosphere or argon atmosphere. In some embodiments, a solvent is added to the reaction mixture. In some embodiments, the solvent forms a second phase, and the cyclopropanation occurs in the aqueous phase. In some embodiments, the heme enzyme is located in the aqueous layer whereas the substrates and/or products occur in an organic layer. Other reaction conditions may be employed in the methods of the invention, depending on the identity of a particular heme enzyme, olefinic substrate, or carbene precursor reagent.

Reactions can be conducted in vivo with intact cells expressing a heme enzyme of the invention. The in vivo reactions can be conducted with any of the host cells used for expression of the heme enzymes, as described herein. A suspension of cells can be formed in a suitable medium supplemented with nutrients (such as mineral micronutrients, glucose and other fuel sources, and the like). Cyclopropanation yields from reactions in vivo can be controlled, in part, by controlling the cell density in the reaction mixtures. Cellular suspensions exhibiting optical densities ranging from about 0.1 to about 50 at 600 nm can be used for cyclopropanation reactions. Other densities can be useful, depending on the cell type, specific heme enzymes, or other factors.

The methods of the invention can be assessed in terms of the diastereoselectivity and/or enantioselectivity of the cyclopropanation reaction—that is, the extent to which the reaction produces a particular isomer, whether a diastereomer or enantiomer. A perfectly selective reaction produces a single isomer, such that the isomer constitutes 100% of the product. As another non-limiting example, a reaction producing a particular enantiomer constituting 90% of the total product can be said to be 90% enantioselective. A reaction producing a particular diastereomer constituting 30% of the total product, meanwhile, can be said to be 30% diastereoselective.

In general, the methods of the invention include reactions that are from about 1% to about 99% diastereoselective. The reactions are from about 1% to about 99% enantioselective. The reaction can be, for example, from about 10% to about 90% diastereoselective, or from about 20% to about 80% diastereoselective, or from about 40% to about 60% diastereoselective, or from about 1% to about 25% diastereoselective, or from about 25% to about 50% diastereoselective, or from about 50% to about 75% diastereoselective. The reaction can be about 10%, 15%, 20%, 25%, 30%, 35%, 40%, 45%, 50%, 55%, 60%, 65%, 70%, 75%, 80%, 85%, 90%, or about 95% diastereoselective. The reaction can be from about 10% to about 90% enantioselective, from about 20% to about 80% enantioselective, or from about 40% to about 60% enantioselective, or from about 1% to about 25% enantioselective, or from about 25% to about 50% enantioselective, or from about 50% to about 75% enantioselective. The reaction can be about 10%, 15%, 20%, 25%, 30%, 35%, 40%, 45%, 50%, 55%, 60%, 65%, 70%, 75%, 80%, 85%, 90%, or about 95% enantioselective. Accordingly some embodiments of the invention provide methods wherein the reaction is at least 30% to at least 90% diastereoselective. In some embodiments, the reaction is at least 30% to at least 90% enantioselective.

As described above, some embodiments of the invention provide methods that include: a) reacting a benzaldehyde of Formula XLIII with a Wittig reagent of Formula XLIV in the presence of a first base in a first solvent to produce a substituted styrene of Formula XLV; b) reacting the styrene of Formula XLV with a diazoester compound of Formula XLVI in the presence of a heme protein catalyst to produce a cyclopropanecarboxylate of Formula XLVIIa; c) hydrolyzing the cyclopropanecarboxylate of Formula XLVIIa with an acid or a second base in a third solvent to produce a cyclopropanecarboxylic acid of Formula XLVIIIa; and f) reacting the cyclopropanecarboxylic acid compound of Formula XLVIIIa with an azide compound in the presence a third base in a fifth solvent to produce an isocyanate intermediate.

Exemplary first solvents used in step-(a) include, but are not limited to, an ester, a nitrile, a hydrocarbon, a cyclic ether, an aliphatic ether, a polar aprotic solvent, and mixtures thereof. The term solvent also includes mixtures of solvents.

Specifically, the first solvent is selected from the group consisting of ethyl acetate, isopropyl acetate, isobutyl acetate, tert-butyl acetate, acetonitrile, propionitrile, tetrahydrofuran, 2-methyl-tetrahydrofuran, 1,4-dioxane, methyl tert-butyl ether, diethyl ether, diisopropyl ether, monoglyme, diglyme, n-hexane, n-heptane, cyclohexane, toluene, xylene, N,N-dimethylformamide, N,N-dimethylacetamide, dimethylsulfoxide, N-methylpyrrolidone, and mixtures thereof; and a most specific solvent is toluene.

In one embodiment, the first base used in step-(a) is an organic or inorganic base. Exemplary organic bases include, but are not limited to, alkyl metals such as methyl lithium, butyl lithium, hexyllithium; alkali metal complexes with amines such as lithium diisopropyl amide; and organic amine bases of formula NR¹⁰¹R¹⁰²R¹⁰³, wherein R¹⁰¹, R¹⁰², and R¹⁰³ are independently hydrogen, C₁₋₆ straight or branched chain alkyl, aryl alkyl, or C₃₋₁₀ single or fused ring optionally substituted, alkylcycloalkyl; or independently R¹⁰¹, R¹⁰², and R¹⁰³ combine with each other to form a C₃₋₇ membered cycloalkyl ring or heterocyclic system containing one or more hetero atoms. Specific organic bases are trimethylamine, dimethyl amine, diethylamine, tert-butyl amine, tributylamine, triethylamine, diisopropylethylamine, pyridine, N-methylmorpholine, 4-(N,N-dimethylamino)pyridine, methyl lithium, butyl lithium, hexyllithium, lithium diisopropyl amide, 1,8-diazabicyclo[5.4.0]undec-7-ene; and most specifically butyl lithium and 1,8-diazabicyclo[5.4.0]undec-7-ene.

Exemplary inorganic bases include, but are not limited to, hydroxides, alkoxides, bicarbonates and carbonates of alkali or alkaline earth metals, and ammonia. Specific inorganic bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide, and more specifically sodium tert-butoxide, sodium isopropoxide and potassium tert-butoxide.

Specific Wittig reagents used in step-(a) are methyl triphenylphosphonium chloride, methyl triphenylphosphonium bromide, methyl triphenylphosphonium iodide, and more specifically methyl triphenylphosphonium bromide.

In one embodiment, the reaction in step-(a) is carried out at a temperature of about −50° C. to about 150° C. for at least 30 minutes, specifically at a temperature of 0° C. to about 100° C. for about 2 hours to about 10 hours, and more specifically at about 35° C. to about 80° C. for about 3 hours to about 6 hours.

The reaction mass containing the substituted styrene compound of Formula XLV obtained in step-(a) may be subjected to usual work up such as a washing, an extraction, a pH adjustment, an evaporation or a combination thereof. The reaction mass may be used directly in the next step or the styrene compound of Formula XLV may be isolated and then used in the next step.

In one embodiment, the styrene compound of Formula XLV is isolated from a suitable solvent by conventional methods such as cooling, seeding, partial removal of the solvent from the solution, by adding an anti-solvent to the solution, evaporation, vacuum distillation, or a combination thereof.

The reaction mass containing the substituted cyclopropanecarboxylate compound of Formula XLVII obtained in step-(b) may be subjected to usual work up such as a washing, an extraction, a pH adjustment, an evaporation or a combination thereof. The reaction mass may be used directly in the next step to produce the cyclopropanecarboxylic acid compound of Formula XLVIII, or the cyclopropanecarboxylate compound of Formula XLVII may be isolated and then used in the next step.

In one embodiment, the cyclopropanecarboxylate compound of Formula XLVII is isolated from a suitable solvent by the methods as described above.

In another embodiment, the solvent used to isolate the cyclopropanecarboxylate compound of Formula XLVII is selected from the group consisting of water, an aliphatic ether, a hydrocarbon solvent, a chlorinated hydrocarbon, and mixtures thereof. Specifically, the solvent is selected from the group consisting of water, toluene, xylene, dichloromethane, diethyl ether, diisopropyl ether, n-heptane, n-pentane, n-hexane, cyclohexane, and mixtures thereof.

### EXAMPLES

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1. In Vitro Enzymatic Synthesis of Cyclopropanes Using CYP102A1 Variants

This example illustrates the purification of CYP102A1 (BM3) enzymes and their use in the production of the compounds of Formulae VII or VIIa.

P450 expression and purification. For the enzymatic transformations, P450 variants were used in purified form. One liter Hyperbrothₐₘₚ was inoculated with an overnight culture (25 mL, TBₐₘₚ) of recombinant E. coli BL21 cells harboring a pCWori or pET22 plasmid encoding the P450 variant under the control of the tac promoter. The cultures were shaken at 200 rpm at 37° C. for roughly 3.5 h or until an optical of density of 1.2-1.8 was reached. The temperature was reduced to 22° C. and the shake rate was reduced to 130-150 rpm for 20 min, then the cultures were induced by adding IPTG and aminolevulinic acid to a final concentration of 0.25 mM and 0.5 mM respectively. The cultures were allowed to continue for another 20 hours at this temperature and shake rate. Cell were harvested by centrifugation (4° C., 15 min, 3,000×g), and the cell pellet was stored at −20° C. or below for at least 2 h. For the purification of 6×His tagged P450s, the thawed cell pellet was resuspended in Ni-NTA buffer A (25 mM Tris.HCl, 200 mM NaCl, 25 mM imidazole, pH 8.0, 4 mL/gcw) and lysed by sonication (2×1 min, output control 5, 50% duty cycle). The lysate was centrifuged at 27,000×g for 20 min at 4° C. to remove cell debris. The collected supernatant was first subjected to a Ni-NTA chromatography step using a Ni Sepharose column (HisTrap-HP, GE healthcare, Piscataway, N.J.). The P450 was eluted from the Ni Sepharose column using 25 mM Tris.HCl, 200 mM NaCl, 300 mM imidazole, pH 8.0. Ni-purified protein was buffer exchanged into 0.1 M phosphate buffer (pH=8.0) using a 30 kDa molecular weight cut-off centrifugal filter. Protein concentrations were determined by CO-assay. For storage, proteins were portioned into 300 μL aliquots and stored at −80° C.

Small-Scale In Vitro Protein Reactions (Anaerobic).

Small-scale (400 μL) reactions were carried out in 2 mL glass crimp vials (Agilent Technologies, San Diego, Calif.). P450 solution (60 μL, 67 μM) was added to an unsealed crimp vial before crimp sealing with a silicone septum. A 12.5 mM solution of sodium dithionite in phosphate buffer (0.1 M, pH=8.0) was degassed by bubbling with argon in a 6 mL crimp-sealed vial. The headspace of the 2 mL vials containing P450 solution were flushed with argon (no bubbling). If multiple reactions were being carried out in parallel, a maximum of 8 vials were connected via cannulae and degassed in series. The buffer/dithionite solution (320 μL) was then added to each reaction vial via syringe, and the gas lines were disconnected from the vials. 10 μL of a stock solution of olefin (400 mM for styrene of Formula V) was added via a glass syringe, followed by 10 μL of a 400 mM stock of ethyldiazoacetate (EDA, example compound of Formula VI) (both stocks in EtOH). The reaction vials were then placed in a tray on a plate shaker and left to shake at 350 rpm for 12 h at room temperature. The final concentrations of the reagents were typically: 10 olefin, 8.7 mM EDA, 10 mM Na₂S₂O₄, and 10 μM P450. The reaction was quenched by the addition of 3 M HCl (25 μL). The vials were uncapped and 1 mL of cyclohexane was added, followed by 20 μL of a 20 mM solution of 2-phenylethanol solution in cyclohexane (internal standard). The mixture was transferred to a 1.5 mL Eppendorf tube and vortexed and centrifuged (10,000×rcf, 30 s). The organic layer was then analyzed by supercritical fluid chromatography (SFC).

The results of the small scale reactions are presented below and demonstrate that a number of CYP102A1 variants are capable of catalyzing formation of the desired cyclopropane carboxylate ethyl ester of Formula VIIa. Specifically, the best variant found in this initial screen of CYP102A1 variants encoded mutations T268A and C400H and gave a modest level of asymmetric induction (18% ee). This can be improved by further engineering, if desired.

TABLE 6

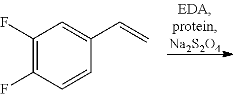

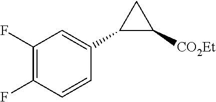 [SMILES: CCOC(=O)[C@@H]1C[C@H]1c1ccc(F)c(F)c1]

| Enzyme | Product/Standard | trans:cis | % ee |
|---|---|---|---|
| HStar | 4.18 | 92:8 | 5.9 |
| HStar I366V | 4.91 | 92:8 | 4.8 |
| T268A C400L heme | 4.63 | 89:11 | −3.1 |
| T268A C400V heme | 4.86 | 89:11 | −2.7 |
| T268A C400H holo | 4.18 | 92:8 | 18 |
| T268A C400D holo | 4.94 | 91:9 | −1.6 |
| T268A C400S holo | 2.50 | 91:8 | N.A. |
| P-I263F | 5.46 | 23:77 | −0.3 |
| BM3-CIS C400S | 3.50 | 20:80 | −8.9 |
| BM3-CIS C400K | 2.60 | 86:14 | N.A. |

### Example 2. In Vitro Enzymatic Synthesis of Cyclopropanes Using CYP119 Variants

CYP119 variants was expressed in BL21(DE3). Seed cultures of 2XYT-amp (50 mL, 100 μg/mL ampicillin) were inoculated from glycerol stocks and grown overnight (200 RPM, 30° C.) in Erlenmeyer flasks (125 mL capacity). The resulting cultures were used to inoculate 1 L of Hyper Broth™ supplemented with ampicillin (100 μg/mL) in Fembach flasks (2.8 L capacity). After inoculation, cultures were grown at 37° C. and 180 RPM for 3.5 hours, then cooled on ice for 10-15 minutes and then induced via addition of IPTG (0.25 mM final concentration) and aminolevulinic acid (0.5 mM final concentration). Induced cultures were then grown overnight at reduced temperature and agitation rate (140 RPM, 25° C.). Following expression, cells were pelleted and frozen at −20° C. until purification.

For purification, frozen cell pellets were resuspended (4 mL/g wet cell weight) in lysis buffer (25 mM Tris, 100 mM NaCl, 30 mM imidazole, lysozyme (0.5 mg/mL), DNaseI (0.02 mg/mL), hemin (1 mg/g wet cell weight), pH 7.5). Cells were disrupted by sonication (3 min, output control 1.5, duty cycle: 5 sec on/10 sec off, Sonicator 3000, Misonix, Inc.). The cell suspension was subsequently incubated for 30 min at 65° C. to precipitate E. coli proteins. To pellet insoluble cell debris, lysates were centrifuged (20,000×g for 30 min at 4° C.). Cleared lysates were then purified loaded onto Ni-NTA columns (5 mL size, HP resin, GE Healthcare) using an AKTAxpress purifier FPLC system (GE healthcare). Target proteins were eluted using a linear gradient from 100% buffer A (25 mM TRIS-HCL, 100 mM NaCl, 10 mM, pH 7.5), 0% buffer B (25 mM Tris, 100 mM NaCl, 300 mM imidazole pH 7.5) to 100% buffer B over 10 column volumes.

Proteins were then pooled, concentrated to 1 mL, and subjected to three 10-fold dilution and concentration steps using centrifugal spin filters (Vivaspin 20, 30 kDA molecular weight cut-off, GE healthcare), each time diluting into fresh buffer (0.1 M KPi pH 8.0).

Small-scale (200 μL) reactions were carried out in 1.7 mL Eppendorf tubes (Agilent Technologies, San Diego, Calif.). P450 solution (5 μL) was added to an unsealed tube before placing in an anaerobic chamber after several cycles of evacuation and refilling with nitrogen gas. A 10.4 mM solution of sodium dithionite in phosphate buffer (0.1 M, pH=8.0) was made by dissolving solid sodium dithionite that had been previously placed in the anaerobic chamber with degassed 0.1 M KPi buffer. The buffer/dithionite solution (320 μL) was then added to each reaction vial in the anaerobic chamber by pipetting. 5 μL of a stock solution of olefin (400 mM for styrene of Formula V) was added via a glass syringe, followed by 5 μL of a 400 mM stock of ethyldiazoacetate (EDA, example compound of Formula VI) (both stocks in methanol). The reaction tubes were then mixed, and allowed to incubate at room temperature in the anaerobic chamber for 12 h at room temperature. The reaction was quenched by removal from the anaerobic chamber, and immediate addition of 3 M HCl (12.5 μL). The vials were uncapped and 0.5 mL of cyclohexane was added, followed by 10 μL of a 20 mM solution of 2-phenylethanol solution in cyclohexane (internal standard). The mixture was vortexed and centrifuged (10,000×rcf, 30 s). The organic layer was then analyzed by supercritical fluid chromatography (SFC).

Results of small-scale reactions are presented below and demonstrate that a number of CYP119 variants are capable of catalyzing formation of the desired cyclopropane carboxylate ethyl ester of Formula VIIa. Specifically, the best variant found in this initial screen encoded a single mutation H315S and gave a modest level of asymmetric induction (31% ee). The opposite enantiomer was obtained for cyclopropanation of the styrene starting material with WT-T268A; WT-T268A gave 40% for the undesired enantiomer.

TABLE 7

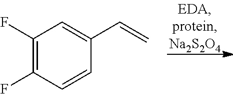

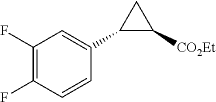 [SMILES: CCOC(=O)[C@@H]1C[C@H]1c1ccc(F)c(F)c1]

| Protein | Product/Standard | trans:cis | % ee |
|---|---|---|---|
| CYP119 C317M | 0.64 | 77:23 | 17 |
| CYP119 C317F | 2.54 | 59:41 | 20 |
| CYP119 C317R | 1.17 | 67:33 | 21 |
| CYP119 C317E | 1.15 | 63:37 | 23 |
| CYP119 C317I | 1.35 | 55:45 | 22 |
| CYP119 C317A | 0.59 | 47:53 | 20 |
| CYP119 C317G | 0.45 | 39:61 | 15 |
| CYP119 C317Y | 1.54 | 62:38 | 25 |
| CYP119 C317Q | 1.77 | 64:36 | 28 |
| CYP119 C317L | 1.60 | 69:31 | 21 |
| CYP119 C317T | 0.85 | 59:41 | 16 |
| CYP119 H315S | 2.69 | 81:19 | 31 |

### Example 3. In Vivo Enzymatic Synthesis of Cyclopropanes Using Cytochrome c, Myoglobin, Globin and P450 Hstar Variants

Expression of Cytochrome c and Variants Thereof.

One liter Hyperbroth (100 μg/mL ampicillin, 20 μg/mL chloramphenicol) was inoculated with an overnight culture of 20 mL LB (100 μg/mL ampicillin, 20 μg/mL chloramphenicol). The overnight culture contained recombinant E. coli BL21-DE3 cells harboring a pET22 plasmid and pEC86 plasmid, encoding the cytochrome c variant under the control of the T7 promoter, and the cytochrome c maturation (ccm) operon under the control of a tet promoter, respectively. The cultures were shaken at 200 rpm at 37° C. for approximately 2 h or until an optical of density of 0.6−0.9 was reached. The flask containing the cells was placed on ice for 30 min. The incubator temperature was reduced to 20° C., maintaining the 200 rpm shake rate. Cultures were induced by adding IPTG and aminolevulinic acid to a final concentration of 20 μM and 200 μM respectively. The cultures were allowed to continue for another 20-24 hours at this temperature and shake rate. Cells were harvested by centrifugation (4° C., 15 min, 3,000×g) to produce a cell pellet.

The procedure for expression of myoglobin (Mb), globin (HGG), BM3 Hstar, and variants thereof was similar to that described for expression of CYP102A1 as described above.

Preparation of Whole Cell Catalysts.

To prepare whole cells for catalysis, the cell pellet prepared in the previous paragraphs was resuspended in M9-N minimal media (M9 media without ammonium chloride) to an optical density (OD₆₀₀) of 60.

Small-Scale In Vivo Reactions (Anaerobic).

Small-scale (400 μL) reactions were carried out in 2 mL glass crimp vials (Agilent Technologies, San Diego, Calif.). Whole cell catalysts (340 μL, OD₆₀₀=60 in M9-N minimal media) were added to an unsealed crimp vial before crimp sealing with a silicone septum. The headspace of the vial was flushed with argon for 10 min (no bubbling). A solution of glucose (40 μL, 250 mM) was added, followed by a solution of olefin of Formula V (10 μL, 800 mM in EtOH; for example, 3,4-difluorostyrene) and a solution of diazo reagent of Formula VI (10 μL, 400 mM in EtOH; for example ethyldiazoacetate, EDA). The reaction vial was left to shake on a plate shaker at 400 rpm for 1 h at room temperature. To quench the reaction, the vial was uncapped and cyclohexane (1 mL) was added, followed by 2-phenylethanol (20 μL, 20 mM in cyclohexane) as an internal standard. The mixture was transferred to a 1.5 mL Eppendorf tube and vortexed and centrifuged (14000×rcf, 5 min). The organic layer was analyzed by gas chromatography (GC) and supercritical fluid chromatography (SFC).

Results of small-scale reactions are presented below and demonstrate that a number of hemoproteins are capable of catalyzing formation of the desired cyclopropane carboxylate ethyl ester of Formula VIIa (the numbers in parentheses correspond to reactions carried out at 4° C. overnight instead of room temperature; the reaction catalyzed by HGG Y29V V68A was carried out using whole cell catalyst with an optical density of 30). Specifically, the best variants found are Hth cyt c encoding the mutations M59A and Q62A, and BM3 Hstar heme domain encoding the mutations H92N and H100N.

TABLE 8

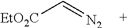

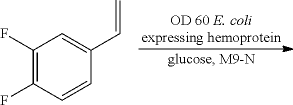

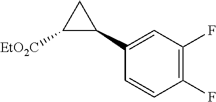

| Whole Cell Catalyst | Product/Standard | trans:cis | % ee |
|---|---|---|---|
| E. coli | 4.7 | 92:8 | 0 |
| Rma CytC M100E | 14.2 | 93:7 | 0 |
| Rma CytC M100S | 13.8 | 95:5 | −16 |
| Rma CytC M100D V75R | 12.7 | 94:6 | 2 |
| Rma CytC M100D V75T | 14.3 | 95:5 | −32 |
| Hth CytC WT | 6.6 | 90:10 | 16 |
| Hth CytC M59A Q62A | 4.4 (6.5) | 89:11 (93:7) | 41 (50) |
| Rgl CytC WT | 6.8 | 90:10 | 0 |
| Mb H64V V68A | 23.5 | 99:1 | −98 |
| HGG Y29V Q50A | 14.0 | 99:1 | −89 |
| Hstar heme | 21.4 (30.0) | 93:7 (94:6) | 12 (10) |
| Hstar H92N H100N heme | 5.8 (8.9) | 93:7 (94:6) | 34 (60) |
| Hstar | 6.4 (8.4) | 91:9 (94:6) | 18 (22) |
| Hstar H92N H100N | 8.8 (15.0) | 92:8 (94:6) | 38 (38) |
| Hstar H100Q | 7.8 (10.9) | 92:8 (94:6) | 26 (52) |
| Hstar H921 H100Q P382Q | 6.3 (18.3) | 91:9 (94:6) | 20 (41) |

### Example 4. Lipase-Catalyzed Resolution of Cyclopropanes Compounds

Lipase enzymes purchased from commercial suppliers or expressed by suitable microbial hosts from suitable plasmids described herein are resuspended, dissolved, or otherwise added to reaction mixtures containing cyclopropane carboxylate compounds of Formulae VII or VIIa in a buffer or solvent mixture similar or identical to those described herein for the purposes of carrying out hemoprotein reactions.

A lipase isolated from Thermomyces lanuginosus (ALMAC lipase kit; AH-45) is added (5% w/v) to a buffered reaction mixture (0.1 M KPi pH 8.0) containing cyclopropane carboxylate esters of Formulae VII or VIIa.

The resulting suspension/solution is then agitated via magnetic stirring at 500 RPM for 12-16 hours at room temperature.

After 12-16 hours, a TLC or HPLC sample is taken to verify that the reaction has produced the desired outcome, namely the hydrolysis of the undesired (S, S) esters of formulae VIIb-d, leading to cyclopropane carboxylic acids VIIIb-d (or salts therefrom).

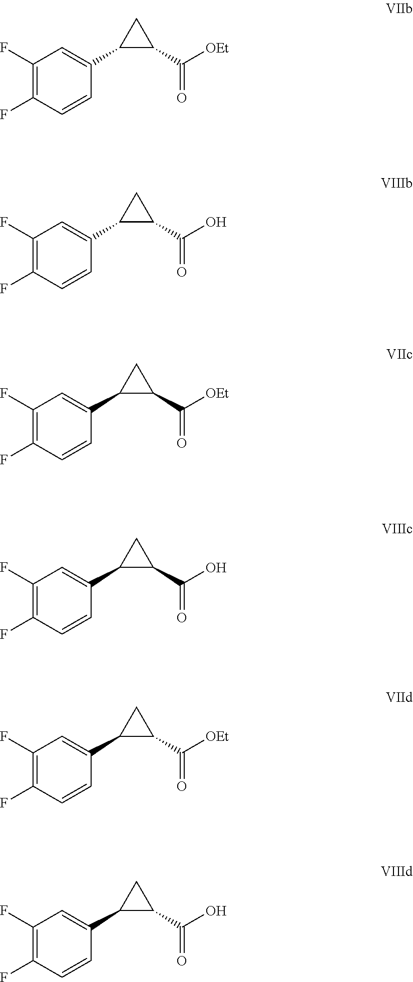 [SMILES: CCOC(=O)[C@H]1C[C@H]1c1ccc(F)c(F)c1 ; O=C(O)[C@H]1C[C@H]1c1ccc(F)c(F)c1 ; CCOC(=O)[C@@H]1C[C@@H]1c1ccc(F)c(F)c1 ; O=C(O)[C@@H]1C[C@@H]1c1ccc(F)c(F)c1 ; CCOC(=O)[C@H]1C[C@@H]1c1ccc(F)c(F)c1 ; O=C(O)[C@H]1C[C@@H]1c1ccc(F)c(F)c1]

### Example 5. Improvement of CYP102A1 Variant-Catalyzed Synthesis of Cyclopropane Compounds

Initial experiments focused on variants of P450-BM3 bearing His, Met, Tyr, or Ala at the proximal position. This set represents a range of possible coordinating heteroatom ligands, and His, Met, and Tyr are found at the axial position of naturally occurring heme proteins such as horseradish peroxidase (HRP), cytochrome c, and catalase. Ala was chosen as well because it is an archetypal small amino acid and may allow a water molecule or hydroxide ion to coordinate to the Fe center. To examine how the different axial ligands affect cyclopropanation activity, each of the four axial mutations were introduced into a P450-BM3 holoenzyme containing the additional mutation T268A. This mutation was previously found to be highly beneficial for cyclopropanation. Four variants, T268A-axX (where “X” denotes the single-letter amino acid code of each axial variant), were expressed as the His-tagged heme domains and purified.

When the reactions of whole E. coli cells expressing the four P450-BM3 variants with olefin of Formula V and ethyl diazoacetate of Formula VI were monitored, it was determined that a CYP102A1 (BM3) variant encoding mutations T268A and C400H (variant T268A-AxH) gave the most of the desired cyclopropane of Formula VIIa.

To create a catalyst more enantioselective than T268A-axH, site-saturation mutagenesis is performed at four active-site positions that had been shown previously to affect selectivity in cyclopropanation or monooxygenation; F87, I263, L437 and T438. The libraries are screened in 96-well plates with whole cells and an oxygen quenching system containing glucose oxidase and catalase in sealed plates. The enantioselectivity of each reaction is determined by chiral supercritical fluid chromatography.

After isolating the most active and enantioselective catalysts from this first round of screening, the catalysts are then subjected to a second round of site-saturation mutagenesis at the positions V78 and L181. The libraries are screened in 96-well plates with whole cells and an oxygen quenching system containing glucose oxidase and catalase in sealed plates. The enantioselectivity of each reaction is determined by chiral supercritical fluid chromatography. The catalysts with the highest activity and enantioselectivity are then chosen for production of the cyclopropane carboxylate ethyl ester of Formula VIIa.

### Example 6. Synthesis of Cyclopropane Compounds Using Myoglobin Variants

Small-scale (400 μL) reactions were carried out in 2 mL glass crimp vials (Agilent Technologies, San Diego, Calif.). Myoglobin or hemin was added to an unsealed crimp vial before crimp sealing with a silicone septum. A 12.5 mM solution of sodium dithionite in phosphate buffer (0.1 M, pH=8.0) was degassed by bubbling with argon in a 6 mL crimp-sealed vial. The headspace of the 2 mL vials containing myoglobin or hemin solution were flushed with argon (no bubbling). The buffer/dithionite solution (300 μL) was then added to each reaction vial via syringe, and the gas lines were disconnected from the vials. 10 μL of a stock solution of olefin (400 mM of 3,4,-difluorostyrene) was added via a glass syringe, followed by 10 μL of a 400 mM stock of ethyldiazoacetate (EDA) (Both stocks in EtOH). The reaction vials were then placed in a tray on a plate shaker and left to shake at 350 rpm for 12 h at room temperature. The final concentrations of the reagents were typically: 10 mM olefin, 8.7 mM EDA, 10 mM Na₂SO₄, and 10 μM myoglobin or 100 μM hemin. The reaction was quenched by the addition of 3 M HCl (25 μL). The vials were uncapped and 1 mL of cyclohexane was added, followed by 20 μL of a 20 mM solution of 2-phenylethanol solution in cyclohexane (internal standard). The mixture was transferred to a 1.5 mL Eppendorf tube and vortexed and centrifuged (10,000×g, 30 s). the organic layer was then analyzed by supercritical fluid chromatography (SFC).

The results of small-scale reactions containing myoglobin or hemin are presented in Table 9. The results demonstrate that either of these is an efficient catalysts of compounds of Formulae VIIa-d. The opposite enantiomer resulting from styrene cyclopropanation was observed when WT-T-268A was used; WT-T268A gave 40% ee for the undesired enantiomer. These results demonstrate that myoglobin and variants thereof are effective asymmetric cyclopropanation catalysts for compounds of Formula VII, though in this specific examples myoglobin preferentially synthesized enantiomer VIIb rather the preferred enantiomer VIIa. Nevertheless it is clearly shown that what is necessary for an enantioselective cyclopropanation catalyst is a chiral protein scaffold and a heme cofactor.

TABLE 9

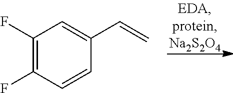

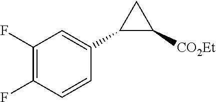 [SMILES: CCOC(=O)[C@@H]1C[C@H]1c1ccc(F)c(F)c1]

| Catalyst | % Yield* | trans:cis | % ee |
|---|---|---|---|
| Sperm whale myoglobzin H64VN68A | 43 | 99:1 | −94 |
| Hemin | 18 | 85:15 | rac |

*based on SFC integration

### Example 7. Creation of Variant Library for the Hemoglobin I from Methylacidophilum infernorum

Based on analysis of the crystal structure of the hemoglobin from Methylacidophilum infernorum (GenBank Accession No. ACD83144), 10 amino acid residues were identified in the distal binding pocket for site-saturation mutagenesis. The 10 amino acids were F28, Y29, L32, F43, Q44, N45, Q50, K53, L54 and V95. Site-saturation mutagenesis was carried out on each of these 10 sites according to the following procedure.

Forward and reverse mutagenic primer pairs were designed to generate 20 different amino acids for each of the 10 amino acid positions. The primers were synthesized and normalized as 5 nmoles by Integrated DNA Technologies, Inc. The primers were diluted with deionized sterile H₂O to approximately 7 μM concentration. A PCR reaction was set up in a total volume of 20 μl, with the final concentrations as follows: 1 U of Pfu turbo DNA polymerase, 1× Pfu turbo buffer, 0.1 mM of dNTP, 20 ng of template DNA and 0.35 μM of forward and reverse primers. The PCR mixture was heated at 95° C. for 5 minutes, then run on 18 cycles of three steps; i) 3 minutes at 95° C., ii) 1 minute at 65° C. and iii) 15 minutes at 68° C., followed by 10 minutes incubation at 72° C. As a template DNA, pET22b vector containing HGbI gene was used. After the PCR reaction was completed, 1 μl of FastDigest DpnI from ThermoFisher Scientific was added to digest the template DNA; incubation was for 3 hours at 37° C. Transformation of the variant DNA library into E. coli was accomplished using NEB® 5-alpha (E coli DH5Alpha) chemically competent cells. For each transformation 3 μl of DNA mixture were used. The three random colonies were picked from each transformation plate and inoculated and grown overnight for submission for Rolling Circle Amplification (RCA) sequencing by Laragen, Inc. to confirm DNA sequences with target mutation(s). Plasmids with the sequences of interest were prepared and used for transformation of E. cloni® EXPRESS BL21(DE3) competent cells purchased from Lucigen Corp. to generate bacterial colonies expressing the HGbI variants.

### Example 8. Preparation of Biocatalysts for Screening of Site-Saturation Variants of Methylacidophilum infernorum

A single colony expressing a HGbI variant was inoculated in 1.5 ml of AthenaES™ hyper broth media containing carbenicillin (100 μg/ml) per well of Axygen Scientific 96-well Deep Well plate. The cells were grown in an INFORS HT plate shaker at 37° C. with 1,000 rpm until optical density (OD₆₀₀) of 1.0 was reached. The expression of the HGbI variants was induced by adding 3 mM of aminolevulinic acid (ALA) and 3 mM Isopropyl β-D-1-thiogalactopyranoside (IPTG), followed by incubation in the INFORS HT plate shaker at 37° C., shaking at 1,000 rpm for 22 hours. To measure the amount of in vivo protein expression, a CO-binding assay was carried out on whole cells. First, microtiter plates containing 200 μl of induced cells per well were centrifuged at 3,000 rpm for 15 minutes, and the cell pellet was resuspended in 200 μl of buffer (100 mM Kpi buffer, pH 7). Subsequently, each sample was transferred to microtiter plate and absorbance spectra were measured at a wavelength between 400 nm and 500 nm using a Tecan Infinite M200Pro reader. Then the microtiter plate was incubated in a CO-chamber at 2 PSI for 1 hour in a fume hood. The CO-chamber was placed under vacuum and flushed with CO gas twice before incubating. The absorbance spectra were re-measured at a wavelength between 400 nm and 500 nm and recorded again. The remaining cells were centrifuged to form a pellet using a Beckman Coulter AVANTI JXN-26 centrifuge at 5,000 rpm for 15 minutes. After discarding the supernatant, the 96-well plate was transferred to an anaerobic chamber from Coy Laboratory Products Inc. Then 190 μl of M9 media that had been pre-purged with nitrogen was added to each well and the cell pellets were resuspended by mixing using an Eppendorf MixMate Vortex Mixer at 1,500 rpm for 3 minutes. The cell suspensions were screened at this stage for cyclopropanation activity.

### Example 9. Screening of Variants of the Hemoglobin I from Methylacidophilum infernorum for Cyclopropanation Activity for the Reaction of 3,4-Difluorostyrene and Ethyl Diazoacetate

Biocatalysts based on hemoglobin I from Methylacidophilum infernorum were prepared as described in Example 8 and screened for ability to catalyze the reaction shown below. See, Teh et al. (FEBS Letters 585(20):3250-8; 2011) for a description of the native protein.

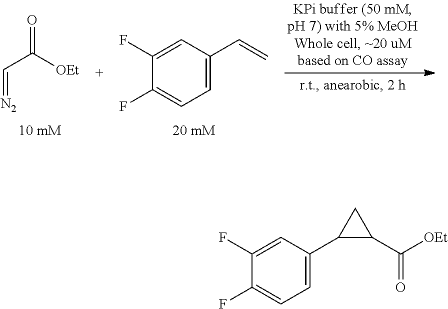

To carry out the cyclopropanation reaction, cell suspensions following growth and induction as in Example 8 were centrifuged using a Beckman Coulter AVANTI JXN-26 centrifuge at 4,000 rpm for 15 minutes. After discarding the supernatant, the 96-well plate was transferred to an anaerobic chamber from Coy Laboratory Products Inc. Cell pellets were resuspended in 190 μl of degassed M9 minimal media using an Eppendorf MixMate Vortex Mixer at 2,000 rpm for 3 minutes. 5 μL of 3,4-difluorostyrene (800 mM, in MeOH solution) was added and incubated for 5 minutes under 1200 rpm. The biocatalysis reaction was initiated by adding 5 μL of ethyl diazoacetate (400 mM, in MeOH). The final concentrations of the reactants were as follows: 20 mM 3,4-difluorostyrene, 10 mM ethyl diazoacetate. The reaction was carried out using an Eppendorf MixMate Vortex Mixer at 1,500 rpm for 30 minutes.

To quench the reaction, the biocatalysis plate was taken out of anaerobic chamber, and the reaction was quenched by the addition of 350 μl of hexane containing 1 mg/mL 1,3,5-trimethoxybenzene (Sigma-Aldrich) as an internal standard, and mixed using Eppendorf MixMate Vortex Mixer for 5 minutes. The 96-well plate was centrifuged in a Beckman Coulter AVANTI JXN-26 centrifuge at 4,000 rpm for 5 minutes. The organic layer was transferred into a 96-well plate (1.1 mL Axygen Scientific 96-well deep well plate, round bottom). Analysis of the product formed was carried out using an Agilent 1100 DAD/RID HPLC system, with a CHIRALCEL OJ-H column (5 um, 4.6*150 mm), isocratic elution with 5% Isopropanol in hexane containing 0.1% acetic acid.

### Example 10. Identification of Variant Hemoglobins from Methylacidophilum infernorum Having Improved Stereoselectivity for the Desired Stereoisomer Trans (1R,2R)-2-(3,4-difluorophenyl)-1-cyclopropanecarboxylic Acid Ethyl Ester

Reactions were carried out in triplicate using the screening procedure described in Example 9. The results of selected variants are shown in FIG. 1.

Where the wild type Hemoglobin I from Methylacidophilum infernorum showed a preference for producing the undesired trans isomer of 2-(3,4-difluorophenyl)-1-cyclopropanecarboxylic acid ethyl ester, several of the variants showed selectivity for producing the desired stereoisomer as the major product As an example of the improvement found, the wild type Hemoglobin I from M. infernorum produced the desired trans (1R,2R)-2-(3,4-difluorophenyl)-1-cyclopropanecarboxylic acid ethyl ester with approximately −30% ee (the negative sign indicates a 30% ee for the undesired stereoisomer) The best single variant, V95F, produced the desired trans stereoisomer with +76% ee, indicating a 75% ee for the desired stereoisomer.

### Example 11. Identification of Double Variant Truncated Hemoglobins from Bacillus subtilis Having Improved Stereoselectivity for the Desired Stereoisomer Trans (1R,2R)-2-(3,4-difluorophenyl)-1-cyclopropanecarboxylic Acid Ethyl Ester

Based on analysis of the crystal structure of the truncated hemoglobin from Bacillus subtilis (GenBank Accession No. AEP90260) 2 amino acid residues were identified in the distal binding pocket for targeted mutagenesis: T45 and Q49. The following 9 targeted double mutants were generated using primer-directed mutagenesis: T45L, Q49L; T45L, Q49F; T45L, Q49A; T45F, Q49L; T45F, Q49F; T45F, Q49A; T45A, Q49L; T45A, Q49F; T45A, Q49A.

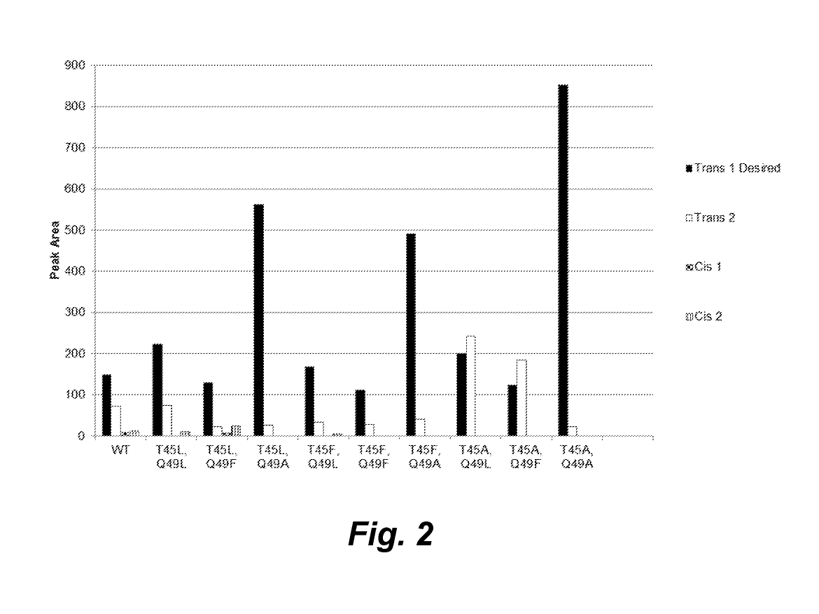

Reactions were carried out in triplicate using the screening procedure described in Example 9. The results of selected variants are shown in FIG. 2. Where the wild type truncated hemoglobin from Bacillus subtilis showed a preference for producing the desired trans isomer of 2-(3,4-difluorophenyl)-1-cyclopropanecarboxylic acid ethyl ester with an ee of approximately +30%, several of the variants showed selectivity for producing the desired stereoisomer as the major product with much higher selectivity. For example, the double variants T45A,Q49A, T45L,Q49A, and T45A,Q45AF produced the desired trans stereoisomer with >+90% ee.

### Example 12. Preparation and Use of Exceptional B. Subtilis Truncated Hemoglobin Variants for Synthesis of Ticagrelor Intermediates

Library Generation and Reaction Screening in 96-Well Format.

For position Y25 of B. subtilis trHb, library was generated by employing the ‘22c-trick’ method (Kille et al. ACS Synth. Biol. 2013, 2, 83). Primers containing NDT, VHG and TGG at the desired positions were mixed in 12:9:1 ratio and then used for PCR using standard QuikChange protocol. The PCR products were gel purified, digested with DpnI, repaired using Gibson Mix™, and then used to transform electrocompetent E. coli BL21(DE3) strain. E. coli μlibraries were cultured in a 96-well plate using LBₐₘₚ (300 μL/well) medium at 37° C., 220 rpm overnight. Hyperbrothₐₘₚ medium (1000 μL/well) was inoculated with the preculture (30 μL/well), and incubated at 37° C., 220 rpm for 3 h. Concurrently, 10 out of the 96 wells were sequenced to ensure its genetic diversity at the desired position (Y25). The remaining of the preculture was used to prepare glycerol stocks of the library, which was stored at −80° C. in 96-well plate. After the indicated time, the 96-well plate expression culture was cooled on ice for 30 min, and then induced with IPTG and 5-aminolevulinic acid to final concentrations of 0.5 mM and 1.0 mM respectively. Protein expression was conducted at 20° C., 220 rpm for 24 h. The cells were pelleted (4000×g, 5 min, 4° C.) and resuspended in nitrogen-free M9-N medium (1 L: 31 g Na₂HPO₄, 15 g KH₂PO₄, 2.5 g NaCl, 0.24 g MgSO₄, 0.01 g CaCl₂; 350 μL/well). The resuspended cells were degassed in the anaerobic chamber, and 3,4-difluorostyrene, EDA, and EtOH were added to each well to final concentrations of 10 mM, 20 mM, and 5% v/v respectively. The plate reaction was shaken in the anaerobic chamber for 1 h, and then taken out to ambient atmosphere. To each well was added 1000 μL of cyclohexane, and the plate was vortexed and centrifuged, and 400 μL aliquots of the organic extracts were transferred to a shallow 96-well plate for analysis.

Analysis of In Vivo Reaction in 96-Well Plate.

Analytical SFC was performed with a Mettler SFC supercritical CO₂ analytical chromatography system using Chiralpak AD column (4.6 mm×25 cm) obtained from Daicel Chemical Industries, Ltd., eluting with 2% isopropanol in liquid CO₂ as the mobile phase (2.5 mL/min flow rate). Observed retention times: 3,4-difluorostyrene-1.41 min, cis cyclopropane product-2.7-2.8 min, trans cyclopropane product (undesired enantiomer)-3.28 min, trans cyclopropane product (desired enantiomer)-4.17 min. Variants that were observed to perform better than the parent Bs trHb T45A Q49A were sequenced and re-screened in small-scale in vivo reaction as described above.

TABLE 10
Enantioselectivity data for Y25X site-saturation library.

| | 1 | 2 | 3 | 4 | 5 | 6 | 7 | 8 | 9 | 10 | 11 | 12 |
|---|---|---|---|---|---|---|---|---|---|---|---|---|
| A | 92.4 | 95.8 | 95.7 | 29.1 | 79.4 | NA | 86.3 | 11.3 | 84.5 | 98.4 | 100.0 | 100.0 |
| B | 100.0 | 93.2 | 95.3 | 86.8 | 82.6 | NA | 89.4 | 2.1 | 92.9 | 94.4 | 96.1 | 100.0 |
| C | 100.0 | 95.7 | 58.5 | 43.6 | 100.0 | NA | 100.0 | 95.9 | 96.6 | 89.6 | 81.6 | 92.1 |
| D | 88.4 | 82.4 | 90.7 | 50.3 | 90.2 | 84.3 | 97.7 | 16.0 | 95.7 | 97.9 | 100.0 | 55.3 |
| E | 87.5 | 71.1 | 89.1 | 32.2 | 82.2 | 81.9 | 93.3 | −1.4 | 88.2 | 95.2 | 93.2 | −6.4 |
| F | 98.6 | 94.6 | 93.9 | 87.3 | 94.7 | 87.1 | 89.9 | 31.7 | 93.4 | 3.5 | 91.8 | 4.9 |
| G | 77.3 | 91.7 | 81.0 | 92.1 | 89.5 | 92.8 | 86.5 | 95.7 | 98.8 | 85.9 | 90.3 | NA |
| H | NA | 100.0 | 100.0 | 97.0 | 92.1 | 44.1 | 10.1 | 94.6 | 76.2 | 94.1 | 11.5 | 92.2 |

Identity of variants: B1, H2, A10 = Y25I; F1, A12, H3 = Y25L

Hit Validation of Bs10 Y25I and Bs10 Y25L.

Small-scale in vivo reactions were performed using the general procedure described in 2016 Brilinta provisional from Caltech, point 97. Deviations from the standard procedure are listed on separate columns on the table below. To investigate the viability of different E. coli strains for the biotransformation, the pET-22b vector harboring the desired gene was used to transform electrocompetent E. coli C41(DE3) and E. coli C43(DE3) (purchased from Lucigen).

TABLE 11

| | | E. coli | | Substrate | | % | % |
|---|---|---|---|---|---|---|---|
| | Protein | strain | OD₆₀₀ | concentration¹ | TTN | trans | ee |
| 1 | Bs10 | BL21 | 30 | A | 5280 | 99 | 96 |
| | | (DE3) | | | | | |
| 2 | Bs10 | BL21 | 30 | B | 2288 | 98 | 81 |
| | | (DE3) | | | | | |
| 3 | Bs10 | C41 (DE3) | 30 | A | 6714 | 99 | 98 |
| 4 | Bs10 | C41 (DE3) | 30 | B | 5208 | 99 | 88 |
| 5 | Bs10 | C43 (DE3) | 30 | A | 6033 | 99 | 97 |
| 6 | Bs10 | C43 (DE3) | 30 | B | 5267 | 99 | 84 |
| 7 | Bs10 Y25I | BL21 | 30 | B | 4133 | 99 | 97 |
| | | (DE3) | | | | | |
| 8 | Bs10 Y25I | C41 (DE3) | 30 | B | 5067 | 99 | 99 |
| 9 | Bs10 Y25I | C43 (DE3) | 30 | B | 6800 | 99 | 99 |
| 10 | Bs10 Y25L | BL21 | 30 | B | 6133 | 99 | 97 |
| | | (DE3) | | | | | |
| 11 | Bs10 Y25L | C41 (DE3) | 30 | B | 8667 | 99 | 98 |
| 12 | Bs10 Y25L | C43 (DE3) | 30 | B | 7467 | 99 | 99 |

¹A = 20 mM styrene, 40 mM ethyl diazoacetate;
B = 50 mM styrene, 100 mM ethyl diazoacetate

Comparing % ee values for Entry 2, Entry 7, and Entry 10, it is apparent that the Bs10 Y25I variant and the Bs10 Y25L variant exhibit superior selectivity as whole-cell catalysts expressed in E. coli BL21(DE3), relative to the parent Bs10. The variants also provide comparable total activity with respect to the parent enzyme (ca. 4000-6000 total turnover number for the transformation). In addition, E. coli strains C₄₁(DE3) and C₄₃(DE3) are superior hosts relative to BL21(DE3) for this reaction. The C₄₁(DE3) and C₄₃(DE3) strains exhibit higher total turnover as shown in Entries 7-12 of Table 11, while also maintaining the enantioselectivity profile of the enzyme.

Performance of Other Globins in In Vivo Cyclopropanation of 3,4-difluorostyrene.

pET-22b vectors harboring neuroglobin (Ngb) F28V F61I H64A and Hell's Gate Globin IV (HgbIV) H71V L93A (UniprotKB accession numbers for WT proteins: Q9NPG2 and B₃DVC₃) were used to transform electrocompetent E. coli BL21(DE3). Performance of these proteins in in vivo cyclopropanation of 3,4-difluorostyrene was assayed following the general procedure for hemoprotein expression and small-scale in vivo reactions (2016 Brilinta provisional from Caltech, point 87, 96, and 97).

TABLE 12

| Protein | OD₆₀₀ | TTN | % trans | % ee |
|---|---|---|---|---|
| Ngb F28V F61I H64A | 30 | 1990 | 90:10 | −47 |
| HgbIV H71V L93A | 30 | 2260 | 93:7 | −41 |

These results show that other natural globins can be engineered to be capable catalysts for the cyclopropanation reaction. Even though initial engineered variants produced predominantly the trans diastereomer of the cyclopropane product, they made the wrong enantiomer in the reaction. However, we contend that these enzymes can be further evolved to produce the desired cyclopropane enantiomer.

### Example 13. Globin-Catalyzed Cyclopropanation Reactions Using Diazoketone Reagents

Protein Purification of Truncated Hemoglobin from Bacillus Subtilis and Hemoglobin I from Methylacidophilum infernorum.

250 mL culture of E coli BL21 DE3 (New England Biolabs) with vector pET22b carrying the gene encoding the truncated hemoglobin from Bacillus subtilis or hemoglobin I from Methylacidophilum infernorum was inoculated. The cells were grown in INNOVA shaker at 37° C. with shaking at 250 rpm until an optical density (OD₆₀₀) of 1.0 was reached. The culture was then induced by adding isopropyl β-D-1-thiogalactopyranoside (IPTG) and aminolevulinic acid (ALA) to a final concentration of 0.5 mM and 1 mM respectively. The culture was incubated at 28° C. with 250 rpm for 22 hours. Cell culture was spun down using Beckman Coulter AVANTI JXN-26 centrifuge at 4,000 rpm for 15 minutes. After discarding the supernatant, the cell pellets were resuspended in 25 mL of lysis buffer (50 mM KPi, 10 mM imidazole, pH 8). Sonication was conducted using Branson digital Ultrasonicator. Crude cell lysates were clarified using Beckman Coulter AVANTI JXN-26 centrifuge at 12,000 rpm for 40 minutes. Further His-tag purification was done following a known procedure (see, Bordeaux, et al. Angew. Chem. Int. Ed. 2015, 54, 1744-1748). Purified enzymes were buffer exchanged into storage buffer (50 mM KPi, pH 8). Enzyme concentration was measured as in previous examples. All variant genes were expressed and purified the same way.

Screening of Globin Variants for Cyclopropanation Activity for the Reaction of 3,4-difluorostyrene and Diazoacetone.

Diazoacetone was prepared according to a published procedure (see, Abid, et al. J. Org. Chem. 2015, 80, 9980-9988). To carry out the cyclopropanation reaction, isolated enzyme samples were transferred into an anaerobic chamber (Coy Laboratory Products Inc.). Enzymes were diluted into degassed buffer (50 mM KPi, 50 mM borate, pH 9) to a final concentration of 10 μM in 1.5 mL Eppendorf tubes. Total volume was 200 μL. Then, 10 μL of 3,4-difluorostyrene (800 mM, in MeOH solution) was added and incubated for 5 minutes under 1200 rpm using MixMate Vortex Mixer. The biocatalysis reaction was initiated by adding 10 μL of diazoacetone solution (400 mM, in MeOH). The final concentrations in the reaction mixture were as follows: 3 μM enzyme, 40 mM 3,4-difluorostyrene, 20 mM diazoacetone.

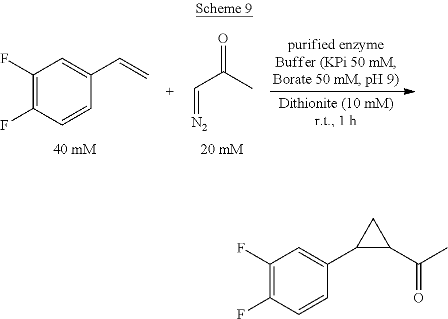

To quench the reaction, the biocatalysis reaction vials were taken out from the anaerobic chamber. The reaction was quenched by addition of 200 μL of hexane containing 0.1% (v/v) hexadecane (Sigma-Aldrich) as internal standard, and quenched mixture was vortexed for 10 seconds using an Eppendorf MixMate Vortex Mixer. The organic layer was transferred into glass HPLC vials with inserts. Analysis of the product mixture was carried out using an Agilent 6890 GC system, Agilent Cyclosil B column (0.25 μm, 0.25 mm*30 m). Chromatography was conducted using a flow rate of 1.4 mL/min and the temperature ramp shown in Table 13.

TABLE 13

| Time (mins) | Ramp (° C./min) | Temperature (° C.) |
|---|---|---|
| 0 | — | 135 |
| 2 | 1 | 165 |
| 32 | 20 | 200 |

Results of the hemoglobin-catalyzed cyclopropanation reactions with diazoacetone are summarized in Table 14. The GC traces included two peaks corresponding to isomeric trans products: an earlier peak at 13.1 min and a later peak at 14.3 min. The results show that the B. subtilis TrHb variants are selective for production of the earlier-eluting trans stereoisomer as the major product. The M. infernorum variant (HG I) is selective for production of the second, later-eluting trans stereoisomer as the major product. In both cases, the amount of trans product was 97% or more of the total product.

TABLE 14

| Variant | TTN | Trans:cis | Trans ee¹ |
|---|---|---|---|
| HG I Q50V/L54A | 2311 | 99:1 | −68.0 |
| BS Wildtype | 0 | — | — |
| BS T45L/Q49A | 511 | 99:1 | 96.1 |
| BS T45F/Q49A | 494 | 97:3 | 96.7 |
| BS T45A/Q49A | 327 | 99:1 | 97.8 |

¹ee calculation: ee = [(earlier trans) − (later trans)]/[(earlier trans) + (later trans)]%

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

INFORMAL SEQUENCE LISTING

```
SEQ ID NO: 1
CYP102A1
Cytochrome P450 (BM3)
Bacillus megaterium
GenBank Accession No. AAA87602
>gi|142798|gb|AAA87602.1| cytochrome P-450:NADPH-P-450 reductase precursor
[Bacillus megaterium]
TIKEMPQPK TFGELKNLPL LNTDKPVQAL MKIADELGEI FKFEAPGRVT RYLSSQRLIK
EACDESRFDK NLSQALKFVR DFAGDGLFTS WTHEKNWKKA HNILLPSFSQ QAMKGYHAMM
VDIAVQLVQK WERLNADEHI EVPEDMTRLT LDTIGLCGFN YRFNSFYRDQ PHPFITSMVR
ALDEAMNKLQ RANPDDPAYD ENKRQFQEDI KVMNDLVDKI IADRKASGEQ SDDLLTHMLN
GKDPETGEPL DDENIRYQII TFLIAGHETT SGLLSFALYF LVKNPHVLQK AAEEAARVLV
DPVPSYKQVK QLKYVGMVLN EALRLWPTAP AFSLYAKEDT VLGGEYPLEK GDELMVLIPQ
LHRDKTIWGD DVEEFRPERF ENPSAIPQHA FKPFGNGQRA CIGQQFALHE ATLVLGMMLK
HFDFEDHTNY ELDIKETLTL KPEGFVVKAK SKKIPLGGIP SPSTEQSAKK VRKKAENAHN
TPLLVLYGSN MGTAEGTARD LADIAMSKGF APQVATLDSH AGNLPREGAV LIVTASYNGH
PPDNAKQFVD WLDQASADEV KGVRYSVFGC GDKNWATTYQ KVPAFIDETL AAKGAENIAD
RGEADASDDF EGTYEEWREH MWSDVAAYFN LDIENSEDNK STLSLQFVDS AADMPLAKMH
GAFSTNVVAS KELQQPGSAR STRHLEIELP KEASYQEGDH LGVIPRNYEG IVNRVTARFG
LDASQQIRLE AEEEKLAHLP LAKTVSVEEL LQYVELQDPV TRTQLRAMAA KTVCPPHKVE
LEALLEKQAY KEQVLAKRLT MLELLEKYPA CEMKFSEFIA LLPSIRPRYY SISSSPRVDE
KQASITVSVV SGEAWSGYGE YKGIASNYLA ELQEGDTITC FISTPQSEFT LPKDPETPLI
MVGPGTGVAP FRGFVQARKQ LKEQGQSLGE AHLYFGCRSP HEDYLYQEEL ENAQSEGIIT
LHTAFSRMPN QPKTYVQHVM EQDGKKLIEL LDQGAHFYIC GDGSQMAPAV EATLMKSYAD
VHQVSEADAR LWLQQLEEKG RYAKDVWAG
SEQ ID NO: 2
CYP102A1
B. megaterium
>gi|281191140|gb|ADA57069.1| NADPH-cytochrome P450 reductase 102A1V9
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HEDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVEGCGDKNWATTYQKVPAFIDETLAAKGAENIAERGEADASDDFEGTYEEWREHMWSDLAAYEN
LDIENSEENASTLSLQFVDSAADMPLAKMHRAFSANVVASKELQKPGSARSTRHLEIELPKEASYQEGDH
LGVIPRNYEGIVNRVATREGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 3
CYP102A1
B. megaterium
>gi|281191138|gb|ADA57068.1| NADPH-cytochrome P450 reductase 102A1V10
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HEDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETFAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 4
CYP102A1
B. megaterium
>gi|281191126|gb|ADA57062.1| NADPH-cytochrome P450 reductase 102A1V4
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQPGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSIRPRYYSISSSPRVDE
KQASITVSVVSGEAWSGYGEYKGIASNYLANLQEGDTITCFVSTPQSGFTLPKDSETPLIMVGPGTGVAP
FRSFVQARKQLKEQGQSLGEAHLYFGCRSPHEDYLYQEELENAQNEGIITLHTAFSRVPNQPKTYVQHVM
EQDGKKLIELLDQGAHFYICGDGSQMAPDVEATLMKSYADVYEVSEADARLWLQQLEEKGRYAKDVWAG
SEQ ID NO: 5
CYP102A1
B. megaterium
>gi|281191124|gb|ADA57061.1| NADPH-cytochrome P450 reductase 102A1V8
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPRVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 6
CYP102A1
B. megaterium
>gi|281191120|gb|ADA57059.1| NADPH-cytochrome P450 reductase 102A1V3
[Bacillus megaterium]
MTIKEMPQPKTFGELKNLPLLNTDKPVQALMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLVQKWERLNADEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
SEQ ID NO: 7
CYP102A1
B. megaterium
>gi|281191118|gb|ADA57058.1| NADPH-cytochrome P450 reductase 102A1V7
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPPEGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNEPKTYVQHVM
EQDGKKLIELLDQGAHFYICGDGSQMAPDVEATLMKSYAEVHQVSEADARLWLQQLEEKGRYAKDVWAG
SEQ ID NO: 8
CYP102A1
B. megaterium
>gi|281191112|gb|ADA57055.1|NADPH-cytochrome P450 reductase 102A1V2
[Bacillus megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
SEQ ID NO: 9
CYP102A1
B. megaterium
>gi|269315992|gb|ACZ37122.1|cytochrome P450:NADPH P450 reductase [Bacillus
megaterium]
MTIKEMPQPKTFGELKNLPLLNTDKPIQTLMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNTDEHI
EVPEDMTRLTLDTIGLCGENYRENSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 10
CYP102A1
B. megaterium
>gi|281191116|gb|ADA57057.1|NADPH-cytochrome P450 reductase 102A1V6
[Bacillus megaterium]
MTIKEMPQPKTFGELKNLPLLNTDKPVQALMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNADEHI
EVPEDMTRLTLDTIGLCGENYRENSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG
SEQ ID NO: 11
CYP102A1
B. megaterium
>gi|281191114|gb|ADA57056.1|NADPH-cytochrome P450 reductase 102A1V5
[Bacillus megaterium]
MTIKEMPQPKTFGELKNLPLLNTDKPVQALMKIADELGEIFKFEAPGRVTRYLSSQRLIKEACDESRFDK
NLSQALKEVRDFAGDGLFTSWTHEKNWKKAHNILLPSFSQQAMKGYHAMMVDIAVQLIQKWERLNADEHI
EVPEDMTRLTLDTIGLCGENYRENSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG
SEQ ID NO: 12
CYP153A6
Mycobacterium sp. EXN-1500
GenBank Accession No.: CAH04396
>gi|519971171embICAH04396.1|cytochrome P450 alkane hydroxylase
[Mycobacterium sp. HXN-1500]
      1 MTEMTVAASD ATNAAYGMAL EDIDVSNPVL FRDNTWHPYF KRLREEDPVH YCKSSMFGPY
    61 WSVTKYRDIM AVETNPKVFS SEAKSGGITI MDDNAAASLP MFIAMDPPKH DVQRKTVSPI
  121 VAPENLATME SVIRQRTADL LDGLPINEEF DWVHRVSIEL TTKMLATLFD FPWDDRAKLT
  181 RWSDVTTALP GGGIIDSEEQ RMAELMECAT YFTELWNQRV NAEPKNDLIS MMAHSESTRH
  241 MAPEEYLGNI VLLIVGGNDT TRNSMTGGVL ALNEFPDEYR KLSANPALIS SMVSEIIRWQ
  301 TPLSHMRRTA LEDIEFGGKH IRQGDKVVMW YVSGNRDPEA IDNPDTFIID RAKPRQHLSF
  361 GFGIHRCVGN RLAELQLNIL WEEILKRWPD PLQIQVLQEP TRVLSPFVKG YESLPVRINA
SEQ ID NO: 13
CYP5013C2
Tetrahymena thermophile
GenBank Accession No.: ABY59989
>gi|164519863|gb|ABY59989.1|cytochrome P450 monooxygenase CYP5013C2
[Tetrahymena thermophila]
      1 MIFELILIAV ALFAYFKIAK PYFSYLKYRK YGKGFYYPIL GEMIEQEQDL KQHADADYSV
    61 HHALDKDPDQ KLFVTNLGTK VKLRLIEPEI IKDFFSKSQY YQKDQTFIQN ITRFLKNGIV
  121 FSEGNTWKES RKLFSPAFHY EYIQKLTPLI NDITDTIFNL AVKNQELKNF DPIAQIQEIT
  181 GRVIIASFFG EVIEGEKFQG LTIIQCLSHI INTLGNQTYS IMYFLFGSKY FELGVTEEHR
  241 KFNKFIAEFN KYLLQKIDQQ IEIMSNELQT KGYIQNPCIL AQLISTHKID EITRNQLFQD
  301 FKTFYIAGMD TTGHLLGMTI YYVSQNKDIY TKLQSEIDSN TDQSAHGLIK NLPYLNAVIK
  361 ETLRYYGPGN ILFDRIAIKD HELAGIPIKK GTIVTPYAMS MQRNSKYYQD PHKYNPSRWL
  421 EKQSSDLHPD ANIPFSAGQR KCIGEQLALL EARIILNKFI KMFDFTCPQD YKLMMNYKFL
  481 SEPVNPLPLQ LTLRKQ
SEQ ID NO: 14
Nonomuraea dietziae
>gi|445067389|gb|AGE14547.1|cytochrome P450 hydroxylase sb8 [Nonomuraea
dietziae]
GenBank Accession No.: AGE14547
VNIDLVDQDHYATEGPPHEQMRWLREHAPVYWHEGEPGFWAVTRHEDVVHVSRHSDLESSARRLALFNEMPEEQR
ELQRMMMLNQDPPEHTRRRSLVNRGETPRTIRALEQHIRDICDDLLDQCSGEGDFVTDLAAPLPLYVICELLGAP
VADRDKIFAWSNRMIGAQDPDYAASPEEGGAAAMEVYAYASELAAQRRAAPRDDIVTKLLQSDENGESLTENEFE
LFVLLLVVAGNETTRNAASGGMLTLFEHPDQWDRLVADPSLAATAADEIVRWVSPVNLFRRTATADLTLGGQQVK
ADDKVVVEYSSANRDASVESDPEVEDIGRSPNPHIGEGGGGAHFCLGNHLAKLELRVLFEQLARREPRMRQTGEA
RRLRSNFINGIKTLPVTLG
SEQ ID NO: 15
CYP2R1
Homo sapiens
GenBank Accession No.: NP 078790
>gi|45267826|ref|NP_078790.21 vitamin D 25-hydroxylase [Homo sapiens]
      1 MWKLWRAEEG AAALGGALFL LLFALGVRQL LKQRRPMGFP PGPPGLPFIG NIYSLAASSE
    61 LPHVYMRKQS QVYGEIFSLD LGGISTVVLN GYDVVKECLV HQSEIFADRP CLPLFMKMTK
  121 MGGLLNSRYG RGWVDHRRLA VNSFRYFGYG QKSFESKILE ETKFFNDAIE TYKGRPFDFK
  181 QLITNAVSNI TNLIIFGERF TYEDTDFQHM IELFSENVEL AASASVFLYN AFPWIGILPF
  241 GKHQQLFRNA AVVYDFLSRL IEKASVNRKP QLPQHFVDAY LDEMDQGKND PSSTFSKENL
  301 IFSVGELIIA GTETTTNVLR WAILFMALYP NIQGQVQKEI DLIMGPNGKP SWDDKCKMPY
  361 TEAVLHEVLR FCNIVPLGIF HATSEDAVVR GYSIPKGTTV ITNLYSVHFD EKYWRDPEVF
  421 HPERFLDSSG YFAKKEALVP FSLGRRHCLG EHLARMEMFL FFTALLQRFH LHFPHELVPD
  481 LKPRLGMTLQ PQPYLICAER R
SEQ ID NO: 16
CYP2R1
Macca mulatta
GenBank Accession No.: NP 001180887
>gi|302565346|ref|NP_001180887.1|vitamin D 25-hydroxylase [Macca mulatta]
      1 MWKLWGGEEG AAALGGALFL LLFALGVRQL LKLRRPMGFP PGPPGLPFIG NIYSLAASAE
    61 LPHVYMRKQS QVYGEIFSLD LGGISTVVLN GYDVVKECLV HQSGIFADRP CLPLFMKMTK
  121 MGGLLNSRYG QGWVEHRRLA VNSFRYFGYG QKSFESKILE ETKFFTDAIE TYKGRPFDFK
  181 QLITSAVSNI TNLIIFGERF TYEDTDFQHM IELFSENVEL AASASVFLYN AFPWIGILPF
  241 GKHQQLFRNA SVVYDFLSRL IEKASVNRKP QLPQHFVDAY FDEMDQGKND PSSTFSKENL
  301 IFSVGELIIA GTETTTNVLR WAILFMALYP NIQGQVQKEI DLIMGPNGKP SWDDKFKMPY
  361 TEAVLHEVLR FCNIVPLGIF HATSEDAVVR GYSIPKGTTV ITNLYSVHFD EKYWRDPEVF
  421 HPERFLDSSG YFAKKEALVP FSLGRRHCLG EQLARMEMFL FFTALLQRFH LHFPHELVPD
  481 LKPRLGMTLQ PQPYLICAER R
SEQ ID NO: 17
CYP2R1
Canis familiaris
GenBank Accession No.: XP_854533
>gi|73988871|ref|XP_854533.1|PREDICTED: vitamin D 25-hydroxylase [Canis
lupus familiaris]
      1 MRGPPGAEAC AAGLGAALLL LLFVLGVRQL LKQRRPAGFP PGPSGLPFIG NIYSLAASGE
    61 LAHVYMRKQS RVYGEIFSLD LGGISAVVLN GYDVVKECLV HQSEIFADRP CLPLFMKMTK
  121 MGGLLNSRYG RGWVDHRKLA VNSFRCFGYG QKSFESKILE ETNFFIDAIE TYKGRPFDLK
  181 QLITNAVSNI TNLIIFGERF TYEDTDFQHM IELFSENVEL AASASVFLYN AFPWIGIIPF
  241 GKHQQLFRNA AVVYDFLSRL IEKASINRKP QSPQHFVDAY LNEMDQGKND PSCTFSKENL
  301 IFSVGELIIA GTETTTNVLR WAILFMALYP NIQGQVQKEI DLIMGPTGKP SWDDKCKMPY
  361 TEAVLHEVLR FCNIVPLGIF HATSEDAVVR GYSIPKGTTV ITNLYSVHFD EKYWRNPEIF
  421 YPERFLDSSG YFAKKEALVP FSLGKRHCLG EQLARMEMFL FFTALLQRFH LHFPHGLVPD
  481 LKPRLGMTLQ PQPYLICAER R
SEQ ID NO: 18
CYP2R1
Mus musculus
GenBank Accession No.: AAI08963
>gi|80477959|gb|AAI08963.1|Cyp2r1 protein [Mus musculus]
      1 MGDEMDQGQN DPLSTFSKEN LIFSVGELII AGTETTTNVL RWAILFMALY PNIQGQVHKE
    61 IDLIVGHNRR PSWEYKCKMP YTEAVLHEVL RFCNIVPLGI FHATSEDAVV RGYSIPKGTT
  121 VITNLYSVHF DEKYWKDPDM FYPERFLDSN GYFTKKEALI PFSLGRRHCL GEQLARMEMF
  181 LFFTSLLQQF HLHFPHELVP NLKPRLGMTL QPQPYLICAE RR
SEQ ID NO: 19
CYP152A6
Bacillus halodurans C-125
GenBank Accession No.: NP_242623
>gi|15614320|ref|NP_242623.1|fatty acid alpha hydroxylase [Bacillus
halodurans C-125]
      1 MKSNDPIPKD SPLDHTMNLM REGYEFLSHR MERFQTDLFE TRVMGQKVLC IRGAEAVKLF
    61 YDPERFKRHR ATPKRIQKSL FGENAIQTMD DKAHLHRKQL FLSMMKPEDE QELARLTHET
  121 WRRVAEGWKK SRPIVLFDEA KRVLCQVACE WAEVPLKSTE IDRRAEDFHA MVDAFGAVGP
  181 RHWRGRKGRR RTERWIQSII HQVRTGSLQA REGSPLYKVS YHRELNGKLL DERMAAIELI
  241 NVLRPIVAIA TFISFAAIAL QEHPEWQERL KNGSNEEFHM FVQEVRRYYP FAPLIGAKVR
  301 KSFTWKGVRF KKGRLVFLDM YGTNHDPKLW DEPDAFRPER FQERKDSLYD FIPQGGGDPT
  361 KGHRCPGEGI TVEVMKTTMD FLVNDIDYDV PDQDISYSLS RMPTRPESGY IMANIERKYE
  421 HA
SEQ ID NO: 20
aryC
Streptomyces parvus
GenBank Accession No.: AFM80022
>gi|392601346|gb|AFM80022.1|cytochrome P450 [Streptomyces parvus]
      1 MYLGGRRGTE AVGESREPGV WEVFRYDEAV QVLGDHRTFS SDMNHFIPEE QRQLARAARG
    61 NFVGIDPPDH TQLRGLVSQA FSPRVTAALE PRIGRLAEQL LDDIVAERGD KASCDLVGEF
  121 AGPLSAIVIA ELFGIPESDH TMIAEWAKAL LGSRPAGELS IADEAAMQNT ADLVRRAGEY
  181 LVHHITERRA RPQDDLTSRL ATTEVDGKRL DDEEIVGVIG MFLIAGYLPA SVLTANTVMA
  241 LDEHPAALAE VRSDPALLPG AIEEVLRWRP PLVRDQRLTT RDADLGGRTV PAGSMVCVWL
  301 ASAHRDPFRF ENPDLFDIHR NAGRHLAFGK GIHYCLGAPL ARLEARIAVE TLLRRFERIE
  361 IPRDESVEFH ESIGVLGPVR LPTTLFARR
SEQ ID NO: 21
CYP101A1
Pseudomonas putida
Uniprot Accession No.: P00183
>sp1P001831CPXA_PSEPU Camphor 5-monooxygenase OS = Pseudomonas putida
GN = camC PE = 1 SV = 2
TTETIQSNANLAPLPPHVPEHLVEDFDMYNPSNLSAGVQEAWAVLQESNVPDLVWTRCNGGHWIATRGQLIREAY
EDYRHFSSECPFIPREAGEAYDFIPTSMDPPEQRQFRALANQVVGMPVVDKLENRIQELACSLIESLRPQGQCNF
TEDYAEPFPIRIFMLLAGLPEEDIPHLKYLTDQMTRPDGSMTFAEAKEALYDYLIPIIEQRRQKPGTDAISIVAN
GQVNGRPITSDEAKRMCGLLLVGGLDTVVNFLSFSMEFLAKSPEHRQELIERPERIPAACEELLRRFSLVADGRI
LTSDYEFHGVQLKKGDQILLPQMLSGLDERENACPMHVDFSRQKVSHTTFGHGSHLCLGQHLARREIIVTLKEWL
TRIPDFSIAPGAQIQHKSGIVSGVQALPLVWDPATTKAV
SEQ ID NO: 22
Homo sapiens
CYP2D7
GenBank Accession No.: AA049806
>gi|37901459|gb|AA049806.1|cytochrome P450 [Homo sapiens]
GLEALVPLA MIVAIFLLLV DLMHRHQRWA ARYPPGPLPL PGLGNLLHVD FQNTPYCFDQ
LRRRFGDVFN LQLAWTPVVV LNGLAAVREA MVTRGEDTAD RPPAPIYQVL GFGPRSQGVI
LSRYGPAWRE QRRFSVSTLR NLGLGKKSLE QWVTEEAACL CAAFADQAGR PFRPNGLLDK
AVSNVIASLT CGRRFEYDDP RFLRLLDLAQ EGLKEESGFL REVLNAVPVL PHIPALAGKV
LRFQKAFLTQ LDELLTEHRM TWDPAQPPRD LTEAFLAKKE KAKGSPESSF NDENLRIVVG
NLFLAGMVTT LTTLAWGLLL MILHLDVQRG RRVSPGCSPI VGTHVCPVRV QQEIDDVIGQ
VRRPEMGDQV HMPYTTAVIH EVQRFGDIVP LGVTHMTSRD IEVQGFRIPK GTTLITNLSS
VLKDEAVWEK PFRFHPEHFL DAQGHFVKPE AFLPFSAGRR ACLGEPLARM ELFLFFTSLL
QHFSFSVAAG QPRPSHSRVV SFLVTPSPYE LCAVPR
SEQ ID NO: 23
Rattus norvegicus
CYPC27
GenBank Accession No.: AAB02287
>gi|1374714|gb|AAB02287.1|cytochrome P450 [Rattus norvegicus]
AVLSRMRLRWALLDTRVMGHGLCPQGARAKAAIPAALRDHESTEGPGTGQDRPRLRSLAELPGPGTLRF
LFQLFLRGYVLHLHELQALNKAKYGPMWTTTEGTRTNVNLASAPLLEQVMRQEGKYPIRDSMEQWKEHRD
HKGLSYGIFITQGQQWYHLRHSLNQRMLKPAEAALYTDALNEVISDFIARLDQVRTESASGDQVPDVAHL
LYHLALEAICYILFEKRVGCLEPSIPEDTATFIRSVGLMFKNSVYVTFLPKWSRPLLPFWKRYMNNWDNI
FSFGEKMIHQKVQEIEAQLQAAGPDGVQVSGYLHFLLTKELLSPQETVGTFPELILAGVDTTSNTLTWAL
YHLSKNPEIQEALHKEVTGVVPFGKVPQNKDFAHMPLLKAVIKETLRLYPVVPTNSRIITEKETEINGFL
FPKNTQFVLCTYVVSRDPSVFPEPESFQPHRWLRKREDDNSGIQHPFGSVPFGYGVRSCLGRRIAELEMQ
LLLSRLIQKYEVVLSPGMGEVKSVSRIVLVPSKKVSLRFLQRQ
SEQ ID NO: 24
CYP2B4
Oryctolagus cuniculus
GenBank Accession No. AAA65840
>gi|164959|gb|AAA65840.1|cytochrome P-450 [Oryctolagus cuniculus]
MEFSLLLLLAFLAGLLLLLFRGHPKAHGRLPPGPSPLPVLGNLLQMDRKGLLRSFLRLRE
KYGDVFTVYLGSRPVVVLCGTDAIREALVDQAEAFSGRGKIAVVDPIFQGYGVIFANGER
WRALRRFSLATMRDFGMGKRSVEERIQEEARCLVEELRKSKGALLDNTLLFHSITSNIIC
SIVEGKREDYKDPVFLRLLDLFFQSFSLISSFSSQVFELFPGFLKHFPGTHRQTYRNLQE
INTFIGQSVEKHRATLDPSNPRDFIDVYLLRMEKDKSDPSSEFHHQNLILTVLSLFFAGT
ETTSTTLRYGELLMLKYPHVTERVQKEIEQVIGSHRPPALDDRAKMPYTDAVIHEIQRLG
DLIPFGVPHTVTKDTQFRGYVIPKNTEVFPVLSSALHDPRYFETPNTFNPGHFLDANGAL
KRNEGFMPFSLGKRICLGEGIARTELFLFFTTILQNFSIASPVPPEDIDLTPRESGVGNV
PPSYQIRFLAR
SEQ ID NO: 25
CYP102A2
Bacillus subtilis
Uniprot Accession No. 008394
>sp10083941CYPD_BACSU Probable bifunctional P-450/NADPH-P450 reductase 1
OS = Bacillus subtilis (strain 168) GN = cypD PE = 1 SV = 2
MKETSPIPQPKTFGPLGNLPLIDKDKPTLSLIKLAEEQGPIFQIHTPAGTTIVVSGHELV
KEVCDEERFDKSIEGALEKVRAFSGDGLFTSWTHEPNWRKAHNILMPTFSQRAMKDYHEK
MVDIAVQLIQKWARLNPNEAVDVPGDMTRLTLDTIGLCGENYRENSYYRETPHPFINSMV
RALDEAMHQMQRLDVQDKLMVRTKRQFRHDIQTMESLVDSIIAERRANGDQDEKDLLARM
LNVEDPETGEKLDDENIRFQIITFLIAGHETTSGLLSFATYFLLKHPDKLKKAYEEVDRV
LTDAAPTYKQVLELTYIRMILNESLRLWPTAPAFSLYPKEDTVIGGKFPITTNDRISVLI
PQLHRDRDAWGKDAEEFRPERFEHQDQVPHHAYKPFGNGQRACIGMQFALHEATLVLGMI
LKYFTLIDHENYELDIKQTLTLKPGDFHIRVQSRNQDAIHADVQAVEKAASDEQKEKTEA
KGTSVIGLNNRPLLVLYGSDTGTAEGVARELADTASLHGVRTETAPLNDRIGKLPKEGAV
VIVTSSYNGKPPSNAGQFVQWLQEIKPGELEGVHYAVFGCGDHNWASTYQYVPRFIDEQL
AEKGATRFSARGEGDVSGDFEGQLDEWKKSMWADAIKAFGLELNENADKERSTLSLQFVR
GLGESPLARSYEASHASIAENRELQSADSDRSTRHIEIALPPDVEYQEGDHLGVLPKNSQ
TNVSRILHRFGLKGTDQVTLSASGRSAGHLPLGRPVSLHDLLSYSVEVQEAATRAQIREL
AAFTVCPPHRRELEELSAEGVYQEQILKKRISMLDLLEKYEACDMPFERFLELLRPLKPR
YYSISSSPRVNPRQASITVGVVRGPAWSGRGEYRGVASNDLAERQAGDDVVMFIRTPESR
FQLPKDPETPIIMVGPGTGVAPFRGFLQARDVLKREGKTLGEAHLYFGCRNDRDFIYRDE
LERFEKDGIVTVHTAFSRKEGMPKTYVQHLMADQADTLISILDRGGRLYVCGDGSKMAPD
VEAALQKAYQAVHGTGEQEAQNWLRHLQDTGMYAKDVWAGI
SEQ ID NO: 26
CYP102A3
Bacillus subtilis
Uniprot Accession No.008336
>sp10083361CYPE_BACSU Probable bifunctional P-450/NADPH-P450 reductase 2
OS = Bacillus subtilis (strain 168) GN = cypE PE = 1 SV = 2
MKQASAIPQPKTYGPLKNLPHLEKEQLSQSLWRIADELGPIFRFDFPGVSSVFVSGHNLV
AEVCDESRFDKNLGKGLQKVREFGGDGLFTSWTHEPNWQKAHRILLPSFSQKAMKGYHSM
MLDIATQLIQKWSRLNPNEEIDVADDMTRLTLDTIGLCGFNYRFNSFYRDSQHPFITSML
RALKEAMNQSKRLGLQDKMMVKTKLQFQKDIEVMNSLVDRMIAERKANPDDNIKDLLSLM
LYAKDPVTGETLDDENIRYQIITFLIAGHETTSGLLSFAIYCLLTHPEKLKKAQEEADRV
LTDDTPEYKQIQQLKYTRMVLNETLRLYPTAPAFSLYAKEDTVLGGEYPISKGQPVTVLI
PKLHRDQNAWGPDAEDFRPERFEDPSSIPHHAYKPFGNGQRACIGMQFALQEATMVLGLV
LKHFELINHTGYELKIKEALTIKPDDFKITVKPRKTAAINVQRKEQADIKAETKPKETKP
KHGTPLLVLYGSNLGTAEGIAGELAAQGRQMGETAETAPLDDYIGKLPEEGAVVIVTASY
NGSPPDNAAGFVEWLKELEEGQLKGVSYAVFGCGNRSWASTYQRIPRLIDDMMKAKGASR
LTEIGEGDAADDFESHRESWENRFWKETMDAFDINEIAQKEDRPSLSIAFLSEATETPVA
KAYGAFEGVVLENRELQTADSTRSTRHIELEIPAGKTYKEGDHIGIMPKNSRELVQRVLS
RFGLQSNHVIKVSGSAHMSHLPMDRPIKVADLLSSYVELQEPASRLQLRELASYTVCPPH
QKELEQLVLDDGIYKEQVLAKRLTMLDFLEDYPACEMPFERFLALLPSLKPRYYSISSSP
KVHANIVSMTVGVVKASAWSGRGEYRGVASNYLAELNTGDAAACFIRTPQSGFQMPDEPE
TPMIMVGPGTGIAPFRGFIQARSVLKKEGSTLGEALLYFGCRRPDHDDLYREELDQAEQE
GLVTIRRCYSRVENESKGYVQHLLKQDSQKLMTLIEKGAHIYVCGDGSQMAPDVEKTLRW
AYETEKGASQEESADWLQKLQDQKRYIKDVWTGN
SEQ ID NO: 27
CYP102A1
B. megaterium DSM 32
Uniprot Accession No. P14779
>sp1P147791CPXB_BACME Bifunctional P-450/NADPH-P450 reductase OS = Bacillus
megaterium GN = cyp102A1 PE = 1 SV = 2
      1 MTIKEMPQPK TFGELKNLPL LNTDKPVQAL MKIADELGEI FKFEAPGRVT RYLSSQRLIK
    61 EACDESRFDK NLSQALKFVR DFAGDGLFTS WTHEKNWKKA HNILLPSFSQ QAMKGYHAMM
  121 VDIAVQLVQK WERLNADEHI EVPEDMTRLT LDTIGLCGFN YRFNSFYRDQ PHPFITSMVR
  181 ALDEAMNKLQ RANPDDPAYD ENKRQFQEDI KVMNDLVDKI IADRKASGEQ SDDLLTHMLN
  241 GKDPETGEPL DDENIRYQII TFLIAGHETT SGLLSFALYF LVKNPHVLQK AAEEAARVLV
  301 DPVPSYKQVK QLKYVGMVLN EALRLWPTAP AFSLYAKEDT VLGGEYPLEK GDELMVLIPQ
  361 LHRDKTIWGD DVEEFRPERF ENPSAIPQHA FKPFGNGQRA CIGQQFALHE ATLVLGMMLK
  421 HFDFEDHTNY ELDIKETLTL KPEGFVVKAK SKKIPLGGIP SPSTEQSAKK VRKKAENAHN
  481 TPLLVLYGSN MGTAEGTARD LADIAMSKGF APQVATLDSH AGNLPREGAV LIVTASYNGH
  541 PPDNAKQFVD WLDQASADEV KGVRYSVFGC GDKNWATTYQ KVPAFIDETL AAKGAENIAD
  601 RGEADASDDF EGTYEEWREH MWSDVAAYFN LDIENSEDNK STLSLQFVDS AADMPLAKMH
  661 GAFSTNVVAS KELQQPGSAR STRHLEIELP KEASYQEGDH LGVIPRNYEG IVNRVTARFG
  721 LDASQQIRLE AEEEKLAHLP LAKTVSVEEL LQYVELQDPV TRTQLRAMAA KTVCPPHKVE
  781 LEALLEKQAY KEQVLAKRLT MLELLEKYPA CEMKFSEFIA LLPSIRPRYY SISSSPRVDE
  841 KQASITVSVV SGEAWSGYGE YKGIASNYLA ELQEGDTITC FISTPQSEFT LPKDPETPLI
  901 MVGPGTGVAP FRGFVQARKQ LKEQGQSLGE AHLYFGCRSP HEDYLYQEEL ENAQSEGIIT
  961 LHTAFSRMPN QPKTYVQHVM EQDGKKLIEL LDQGAHFYIC GDGSQMAPAV EATLMKSYAD
1021 VHQVSEADAR LWLQQLEEKG RYAKDVWAG
SEQ ID NO: 28
CYP102A5
B. cereus ATCC14579
GenBank Accession No. AAP10153
>gi|29896875|gb|AAP10153.1|NADPH-cytochrome P450 reductase [Bacillus
cereus ATCC 145791
      1 MEKKVSAIPQ PKTYGPLGNL PLIDKDKPTL SFIKIAEEYG PIFQIQTLSD TIIVVSGHEL
    61 VAEVCDETRF DKSIEGALAK VRAFAGDGLF TSETHEPNWK KAHNILMPTF SQRAMKDYHA
  121 MMVDIAVQLV QKWARLNPNE NVDVPEDMTR LTLDTIGLCG FNYRFNSFYR ETPHPFITSM
  181 TRALDEAMHQ LQRLDIEDKL MWRTKRQFQH DIQSMFSLVD NIIAERKSSG DQEENDLLSR
  241 MLNVPDPETG EKLDDENIRF QIITFLIAGH ETTSGLLSFA IYFLLKNPDK LKKAYEEVDR
  301 VLTDPTPTYQ QVMKLKYMRM ILNESLRLWP TAPAFSLYAK EDTVIGGKYP IKKGEDRISV
  361 LIPQLHRDKD AWGDNVEEFQ PERFEELDKV PHHAYKPFGN GQRACIGMQF ALHEATLVMG
  421 MLLQHFELID YQNYQLDVKQ TLTLKPGDFK IRILPRKQTI SHPTVLAPTE DKLKNDEIKQ
  481 HVQKTPSIIG ADNLSLLVLY GSDTGVAEGI ARELADTASL EGVQTEVVAL NDRIGSLPKE
  541 GAVLIVTSSY NGKPPSNAGQ FVQWLEELKP DELKGVQYAV FGCGDHNWAS TYQRIPRYID
  601 EQMAQKGATR FSKRGEADAS GDFEEQLEQW KQNMWSDAMK AFGLELNKNM EKERSTLSLQ
  661 FVSRLGGSPL ARTYEAVYAS ILENRELQSS SSDRSTRHIE VSLPEGATYK EGDHLGVLPV
  721 NSEKNINRIL KRFGLNGKDQ VILSASGRSI NHIPLDSPVS LLALLSYSVE VQEAATRAQI
  781 REMVTFTACP PHKKELEALL EEGVYHEQIL KKRISMLDLL EKYEACEIRF ERFLELLPAL
  841 KPRYYSISSS PLVAHNRLSI TVGVVNAPAW SGEGTYEGVA SNYLAQRHNK DEIICFIRTP
  901 QSNFELPKDP ETPIIMVGPG TGIAPFRGFL QARRVQKQKG MNLGQAHLYF GCRHPEKDYL
  961 YRTELENDER DGLISLHTAF SRLEGHPKTY VQHLIKQDRI NLISLLDNGA HLYICGDGSK
1021 MAPDVEDTLC QAYQEIHEVS EQEARNWLDR VQDEGRYGKD VWAGI
SEQ ID NO: 29
CYP102A7
B. licheniformis ATTC1458
GenBank Accession No. YP 079990
>gi|520811991reflYP_079990.1|cytochrome P450 / NADPH-ferrihemoprotein
reductase [Bacillus licheniformis DSM 13 =ATCC 145801
      1 MNKLDGIPIP KTYGPLGNLP LLDKNRVSQS LWKIADEMGP IFQFKFADAI GVFVSSHELV
    61 KEVSEESRFD KNMGKGLLKV REFSGDGLFT SWTEEPNWRK AHNILLPSFS QKAMKGYHPM
  121 MQDIAVQLIQ KWSRLNQDES IDVPDDMTRL TLDTIGLCGF NYRFNSFYRE GQHPFIESMV
  181 RGLSEAMRQT KRFPLQDKLM IQTKRRFNSD VESMFSLVDR IIADRKQAES ESGNDLLSLM
  241 LHAKDPETGE KLDDENIRYQ IITFLIAGHE TTSGLLSFAI YLLLKHPDKL KKAYEEADRV
  301 LTDPVPSYKQ VQQLKYIRMI LNESIRLWPT APAFSLYAKE ETVIGGKYLI PKGQSVTVLI
  361 PKLHRDQSVW GEDAEAFRPE RFEQMDSIPA HAYKPFGNGQ RACIGMQFAL HEATLVLGMI
  421 LQYFDLEDHA NYQLKIKESL TLKPDGFTIR VRPRKKEAMT AMPGAQPEEN GRQEERPSAP
  481 AAENTHGTPL LVLYGSNLGT AEEIAKELAE EAREQGFHSR TAELDQYAGA IPAEGAVIIV
  541 TASYNGNPPD CAKEFVNWLE HDQTDDLRGV KYAVFGCGNR SWASTYQRIP RLIDSVLEKK
  601 GAQRLHKLGE GDAGDDFEGQ FESWKYDLWP LLRTEFSLAE PEPNQTETDR QALSVEFVNA
  661 PAASPLAKAY QVFTAKISAN RELQCEKSGR STRHIEISLP EGAAYQEGDH LGVLPQNSEV
  721 LIGRVFQRFG LNGNEQILIS GRNQASHLPL ERPVHVKDLF QHCVELQEPA TRAQIRELAA
  781 HTVCPPHQRE LEDLLKDDVY KDQVLNKRLT MLDLLEQYPA CELPFARFLA LLPPLKPRYY
  841 SISSSPQLNP RQTSITVSVV SGPALSGRGH YKGVASNYLA GLEPGDAISC FIREPQSGFR
  901 LPEDPETPVI MVGPGTGIAP YRGFLQARRI QRDAGVKLGE AHLYFGCRRP NEDFLYRDEL
  961 EQAEKDGIVH LHTAFSRLEG RPKTYVQDLL REDAALLIHL LNEGGRLYVC GDGSRMAPAV
1021 EQALCEAYRI VQGASREESQ SWLSALLEEG RYAKDVWDGG VSQHNVKADC IART
SEQ ID NO: 30
CYPX
B. thuringiensis serovar konkukian
str.97-27
GenBank Accession No. YP 037304
>gi|494800991reflYP_037304.1|NADPH-cytochrome P450 reductase [Bacillus
thuringiensis serovar konkukian str. 97-271
      1 MDKKVSAIPQ PKTYGPLGNL PLIDKDKPTL SFIKLAEEYG PIFQIQTLSD TIIVVSGHEL
    61 VAEVCDETRF DKSIEGALAK VRAFAGDGLF TSETDEPNWK KAHNILMPTF SQRAMKDYHA
  121 MMVDIAVQLV QKWARLNPNE NVDVPEDMTR LTLDTIGLCG FNYRFNSFYR ETPHPFITSM
  181 TRALDEAMHQ LQRLDIEDKL MWRTKRQFQH DIQSMFSLVD NIIAERKSSE NQEENDLLSR
  241 MLNVQDPETG EKLDDENIRF QIITFLIAGH ETTSGLLSFA IYFLLKNPDK LKKAYEEVDR
  301 VLTDSTPTYQ QVMKLKYIRM ILNESLRLWP TAPAFSLYAK EDTVIGGKYP IKKGEDRISV
  361 LIPQLHRDKD AWGDDVEEFQ PERFEELDKV PHHAYKPFGN GQRACIGMQF ALHEATLVMG
  421 MLLQHFEFID YEDYQLDVKQ TLTLKPGDFK IRIVPRNQTI SHTTVLAPTE EKLKKHEIKK
  481 QVQKTPSIIG ADNLSLLVLY GSDTGVAEGI ARELADTASL EGVQTEVVAL NDRIGSLPKE
  541 GAVLIVTSSY NGKPPSNAGQ FVQWLEELKP DELKGVQYAV FGCGDHNWAS TYQRIPRYID
  601 EQMAQKGATR FSTRGEADAS GDFEEQLEQW KQSMWSDAMK AFGLELNKNM EKERSTLSLQ
  661 FVSRLGGSPL ARTYEAVYAS ILENRELQSS SSERSTRHIE ISLPEGATYK EGDHLGVLPI
  721 NNEKNVNRIL KRFGLNGKDQ VILSASGRSV NHIPLDSPVR LYDLLSYSVE VQEAATRAQI
  781 REMVTFTACP PHKKELESLL EDGVYQEQIL KKRISMLDLL EKYEACEIRF ERFLELLPAL
  841 KPRYYSISSS PLVAQDRLSI TVGVVNAPAW SGEGTYEGVA SNYLAQRHNK DEIICFIRTP
  901 QSNFQLPENP ETPIIMVGPG TGIAPFRGFL QARRVQKQKG MKVGEAHLYF GCRHPEKDYL
  961 YRTELENDER DGLISLHTAF SRLEGHPKTY VQHVIKEDRI HLISLLDNGA HLYICGDGSK
1021 MAPDVEDTLC QAYQEIHEVS EQEARNWLDR LQEEGRYGKD VWAGI
SEQ ID NO: 31
CYP102E1
R. metallidurans CH34
GenBank Accession No. YP 585608
>gi|943123981reflYP_585608.1|putative bifunctional P-450:NADPH-P450
reductase 2 [Cupriavidus metallidurans CH34]
      1 MSTATPAAAL EPIPRDPGWP IFGNLFQITP GEVGQHLLAR SRHHDGIFEL DFAGKRVPFV
    61 SSVALASELC DATRFRKIIG PPLSYLRDMA GDGLFTAHSD EPNWGCAHRI LMPAFSQRAM
  121 KAYFDVMLRV ANRLVDKWDR QGPDADIAVA DDMTRLTLDT IALAGFGYDF ASFASDELDP
  181 FVMAMVGALG EAMQKLTRLP IQDRFMGRAH RQAAEDIAYM RNLVDDVIRQ RRVSPTSGMD
  241 LLNLMLEARD PETDRRLDDA NIRNQVITFL IAGHETTSGL LTFALYELLR NPGVLAQAYA
  301 EVDTVLPGDA LPVYADLARM PVLDRVLKET LRLWPTAPAF AVAPFDDVVL GGRYRLRKDR
  361 RISVVLTALH RDPKVWANPE RFDIDRFLPE NEAKLPAHAY MPFGQGERAC IGRQFALTEA
  421 KLALALMLRN FAFQDPHDYQ FRLKETLTIK PDQFVLRVRR RRPHERFVTR QASQAVADAA
  481 QTDVRGHGQA MTVLCASSLG TARELAEQIH AGAIAAGFDA KLADLDDAVG VLPTSGLVVV
  541 VAATYNGRAP DSARKFEAML DADDASGYRA NGMRLALLGC GNSQWATYQA FPRRVFDFFI
  601 TAGAVPLLPR GEADGNGDFD QAAERWLAQL WQALQADGAG TGGLGVDVQV RSMAAIRAET
  661 LPAGTQAFTV LSNDELVGDP SGLWDFSIEA PRTSTRDIRL QLPPGITYRT GDHIAVWPQN
  721 DAQLVSELCE RLDLDPDAQA TISAPHGMGR GLPIDQALPV RQLLTHFIEL QDVVSRQTLR
  781 ALAQATRCPF TKQSIEQLAS DDAEHGYATK VVARRLGILD VLVEHPAIAL TLQELLACTV
  841 PMRPRLYSIA SSPLVSPDVA TLLVGTVCAP ALSGRGQFRG VASTWLQHLP PGARVSASIR
  901 TPNPPFAPDP DPAAPMLLIG PGTGIAPFRG FLEERALRKM AGNAVTPAQL YFGCRHPQHD
  961 WLYREDIERW AGQGVVEVHP AYSVVPDAPR YVQDLLWQRR EQVWAQVRDG ATIYVCGDGR
1021 RMAPAVRQTL IEIGMAQGGM TDKAASDWFG GLVAQGRYRQ DVFN
SEQ ID NO: 32
CYP505X
A. fumigatus Af293
GenBank Accession No. EAL92660
>gi|66852335|gb|EAL92660.1|P450 family fatty acid hydroxylase, putative
[Aspergillus fumigatus Af293]
      1 MSESKTVPIP GPRGVPLLGN IYDIEQEVPL RSINLMADQY GPIYRLTTFG WSRVFVSTHE
    61 LVDEVCDEER FTKVVTAGLN QIRNGVHDGL FTANFPGEEN WAIAHRVLVP AFGPLSIRGM
  121 FDEMYDIATQ LVMKWARHGP TVPIMVTDDF TRLTLDTIAL CAMGTRFNSF YHEEMHPFVE
  181 AMVGLLQGSG DRARRPALLN NLPTSENSKY WDDIAFLRNL AQELVEARRK NPEDKKDLLN
  241 ALILGRDPKT GKGLTDESII DNMITFLIAG HETTSGLLSF LFYYLLKTPN AYKKAQEEVD
  301 SVVGRRKITV EDMSRLPYLN AVMRETLRLR STAPLIAVHA HPEKNKEDPV TLGGGKYVLN
  361 KDEPIVIILD KLHRDPQVYG PDAEEFKPER MLDENFEKLP KNAWKPFGNG MRACIGRPFA
  421 WQEALLVVAI LLQNFNFQMD DPSYNLHIKQ TLTIKPKDFH MRATLRHGLD ATKLGIALSG
  481 SADRAPPESS GAASRVRKQA TPPAGQLKPM HIFFGSNTGT CETFARRLAD DAVGYGFAAD
  541 VQSLDSAMQN VPKDEPVVFI TASYEGQPPD NAAHFFEWLS ALKENELEGV NYAVFGCGHH
  601 DWQATFHRIP KAVNQLVAEH GGNRLCDLGL ADAANSDMFT DFDSWGESTF WPAITSKFGG
  661 GKSDEPKPSS SLQVEVSTGM RASTLGLQLQ EGLVIDNQLL SAPDVPAKRM IRFKLPSDMS
  721 YRCGDYLAVL PVNPTSVVRR AIRRFDLPWD AMLTIRKPSQ APKGSTSIPL DTPISAFELL
  781 STYVELSQPA SKRDLTALAD AAITDADAQA ELRYLASSPT RFTEEIVKKR MSPLDLLIRY
  841 PSIKLPVGDF LAMLPPMRVR QYSISSSPLA DPSECSITFS VLNAPALAAA SLPPAERAEA
  901 EQYMGVASTY LSELKPGERA HIAVRPSHSG FKPPMDLKAP MIMACAGSGL APFRGFIMDR
  961 AEKIRGRRSS VGADGQLPEV EQPAKAILYV GCRTKGKDDI HATELAEWAQ LGAVDVRWAY
1021 SRPEDGSKGR HVQDLMLEDR EELVSLFDQG ARIYVCGSTG VGNGVRQACK DIYLERRRQL
1081 RQAARERGEE VPAEEDEDAA AEQFLDNLRT KERYATDVFT
SEQ ID NO: 33
CYP505A8
A. nidulans FGSC A4
GenBank Accession No. EAA58234
>gi|40739044|gb|EAA58234.1|hypothetical protein AN6835.2 [Aspergillus
nidulans FGSC A4]
      1 MAEIPEPKGL PLIGNIGTID QEFPLGSMVA LAEEHGEIYR LRFPGRTVVV VSTHALVNET
    61 CDEKRFRKSV NSALAHVREG VHDGLFTAKM GEVNWEIAHR VLMPAFGPLS IRGMFDEMHD
  121 IASQLALKWA RYGPDCPIMV TDDFTRLTLD TLALCSMGYR FNSYYSPVLH PFIEAMGDFL
  181 TEAGEKPRRP PLPAVFFRNR DQKFQDDIAV LRDTAQGVLQ ARKEGKSDRN DLLSAMLRGV
  241 DSQTGQKMTD ESIMDNLITF LIAGHETTSG LLSFVFYQLL KHPETYRTAQ QEVDNVVGQG
  301 VIEVSHLSKL PYINSVLRET LRLNATIPLF TVEAFEDTLL AGKYPVKAGE TIVNLLAKSH
  361 LDPEVYGEDA LEFKPERMSD ELFNARLKQF PSAWKPFGNG MRACIGRPFA WQEALLVMAM
  421 LLQNFDFSLA DPNYDLKFKQ TLTIKPKDMF MKARLRHGLT PTTLERRLAG LAVESATQDK
  481 IVTNPADNSV TGTRLTILYG SNSGTCETLA RRIAADAPSK GFHVMRFDGL DSGRSALPTD
  541 HPVVIVTSSY EGQPPENAKQ FVSWLEELEQ QNESLQLKGV DFAVFGCFKE WAQTFHRIPK
  601 LVDSLLEKLG GSRLTDLGLA DVSTDELFST FETWADDVLW PRLVAQYGAD GKTQAHGSSA
  661 GHEAASNAAV EVTVSNSRTQ ALRQDVGQAM VVETRLLTAE SEKERRKKHL EIRLPDGVSY
  721 TAGDYLAVLP INPPETVRRA MRQFKLSWDA QITIAPSGPT TALPTDGPIA ANDIFSTYVE
  781 LSQPATRKDL RIMADATTDP DVQKILRTYA NETYTAEILT KSISVLDILE QHPAIDLPLG
  841 TFLLMLPSMR MRQYSISSSP LLTPTTATIT ISVLDAPSRS RSNGSRHLGV ATSYLDSLSV
  901 GDHLQVTVRK NPSSGFRLPS EPETTPMICI AAGSGIAPFR AFLQERAVMM EQDKDRKLAP
  961 ALLFFGCRAP GIDDLYREQL EEWQARGVVD ARWAFSRQSD DTKGCRHVDD RILADREDVV
1021 KLWRDGARVY VCGSGALAQS VRSAMVTVLR DEMETTGDGS DNGKAEKWFD EQRNVRYVMD
1081 VFD
SEQ ID NO: 34
CYP505A3
A. oryzae ATCC42149
Uniprot Accession No. Q2U4F1
>gi|121928062|sp|Q2U4F1|Q2U4F1_ASPOR Cytochrome P450
      1 MRQNDNEKQI CPIPGPQGLP FLGNILDIDL DNGTMSTLKI AKTYYPIFKF TFAGETSIVI
    61 NSVALLSELC DETRFHKHVS FGLELLRSGT HDGLFTAYDH EKNWELAHRL LVPAFGPLRI
  121 REMFPQMHDI AQQLCLKWQR YGPRRPLNLV DDFTRTTLDT IALCAMGYRF NSFYSEGDFH
  181 PFIKSMVRFL KEAETQATLP SFISNLRVRA KRRTQLDIDL MRTVCREIVT ERRQTNLDHK
  241 NDLLDTMLTS RDSLSGDALS DESIIDNILT FLVAGHETTS GLLSFAVYYL LTTPDAMAKA
  301 AHEVDDVVGD QELTIEHLSM LKYLNAILRE TLRLMPTAPG FSVTPYKPEI IGGKYEVKPG
  361 DSLDVFLAAV HRDPAVYGSD ADEFRPERMS DEHFQKLPAN SWKPFGNGKR SCIGRAFAWQ
  421 EALMILALIL QSFSLNLVDR GYTLKLKESL TIKPDNLWAY ATPRPGRNVL HTRLALQTNS
  481 THPEGLMSLK HETVESQPAT ILYGSNSGTC EALAHRLAIE MSSKGRFVCK VQPMDAIEHR
  541 RLPRGQPVII ITGSYDGRPP ENARHFVKWL QSLKGNDLEG IQYAVFGCGL PGHHDWSTTF
  601 YKIPTLIDTI MAEHGGARLA PRGSADTAED DPFAELESWS ERSVWPGLEA AFDLVRHNSS
  661 DGTGKSTRIT IRSPYTLRAA HETAVVHQVR VLTSAETTKK VHVELALPDT INYRPGDHLA
  721 ILPLNSRQSV QRVLSLFQIG SDTILYMTSS SATSLPTDTP ISAHDLLSGY VELNQVATPT
  781 SLRSLAAKAT DEKTAEYLEA LATDRYTTEV RGNHLSLLDI LESYSVPSIE IQHYIQMLPL
  841 LRPRQYTISS SPRLNRGQAS LTVSVMERAD VGGPRNCAGV ASNYLASCTP GSILRVSLRQ
  901 ANPDFRLPDE SCSHPIIMVA AGSGIAPFRA FVQERSVRQK EGIILPPAFL FFGCRRADLD
  961 DLYREELDAF EEQGVVTLFR AFSRAQSESH GCKYVQDLLW MERVRVKTLW GQDAKVFVCG
1021 SVRMNEGVKA IISKIVSPTP TEELARRYIA ETFI
SEQ ID NO: 35
CYPX
A. oryzae ATCC42149
Uniprot Accession No. Q2UNA2
>gi|121938553|sp|Q2UNA2|Q2UNA2_ASPOR Cytochrome P450
      1 MSTPKAEPVP IPGPRGVPLM GNILDIESEI PLRSLEMMAD TYGPIYRLTT FGFSRCMISS
    61 HELAAEVFDE ERFTKKIMAG LSELRHGIHD GLFTAHMGEE NWEIAHRVLM PAFGPLNIQN
  121 MFDEMHDIAT QLVMKWARQG PKQKIMVTDD FTRLTLDTIA LCAMGTRFNS FYSEEMHPFV
  181 DAMVGMLKTA GDRSRRPGLV NNLPTTENNK YWEDIDYLRN LCKELVDTRK KNPTDKKDLL
  241 NALINGRDPK TGKGMSYDSI IDNMITFLIA GHETTSGSLS FAFYNMLKNP QAYQKAQEEV
  301 DRVIGRRRIT VEDLQKLPYI TAVMRETLRL TPTAPAIAVG PHPTKNHEDP VTLGNGKYVL
  361 GKDEPCALLL GKIQRDPKVY GPDAEEFKPE RMLDEHFNKL PKHAWKPFGN GMRACIGRPF
  421 AWQEALLVIA MLLQNFNFQM DDPSYNIQLK QTLTIKPNHF YMRAALREGL DAVHLGSALS
  481 ASSSEHADHA AGHGKAGAAK KGADLKPMHV YYGSNTGTCE AFARRLADDA TSYGYSAEVE
  541 SLDSAKDSIP KNGPVVFITA SYEGQPPDNA AHFFEWLSAL KGDKPLDGVN YAVFGCGHHD
  601 WQTTFYRIPK EVNRLVGENG ANRLCEIGLA DTANADIVTD FDTWGETSFW PAVAAKFGSN
  661 TQGSQKSSTF RVEVSSGHRA TTLGLQLQEG LVVENTLLTQ AGVPAKRTIR FKLPTDTQYK
  721 CGDYLAILPV NPSTVVRKVM SRFDLPWDAV LRIEKASPSS SKHISIPMDT QVSAYDLFAT
  781 YVELSQPASK RDLAVLADAA AVDPETQAEL QAIASDPARF AEISQKRISV LDLLLQYPSI
  841 NLAIGDFVAM LPPMRVRQYS ISSSPLVDPT ECSITFSVLK APSLAALTKE DEYLGVASTY
  901 LSELRSGERV QLSVRPSHTG FKPPTELSTP MIMACAGSGL APFRGFVMDR AEKIRGRRSS
  961 GSMPEQPAKA ILYAGCRTQG KDDIHADELA EWEKIGAVEV RRAYSRPSDG SKGTHVQDLM
1021 MEDKKELIDL FESGARIYVC GTPGVGNAVR DSIKSMFLER REEIRRIAKE KGEPVSDDDE
1081 ETAFEKFLDD MKTKERYTTD IFA
SEQ ID NO: 36
CYP505A1
F. oxysporum
Uniprot Accession No. Q9Y8G7
>gi|22653677|sp|Q9Y8G7.1|C505_FUSOX RecName: Full = Bifunctional P-450:NADPH-
P450 reductase; AltName: Full = Cytochrome P450foxy; AltName: Full = Fatty acid
omega-hydroxylase; Includes: RecName: Full = Cytochrome P450 505; Includes:
RecName: Full = NADPH--cytochrome P450 reductase
      1 maesvpipep pgyplignlg eftsnplsdl nrladtygpi frlrlgakap ifvssnslin
    61 evcdekrfkk tlksvlsqvr egvhdglfta fedepnwgka hrilvpafgp lsirgmfpem
  121 hdiatqlcmk farhgprtpi dtsdnftrla ldtlalcamd frfysyykee lhpfieamgd
  181 fltesgnrnr rppfapnfly raanekfygd ialmksvade vvaarkasps drkdllaaml
  241 ngvdpqtgek lsdenitnql itfliaghet tsgtlsfamy qllknpeays kvqkevdevv
  301 grgpvlvehl tklpyisavl retlrinspi tafgleaidd tflggkylvk kgeivtalls
  361 rghvdpvvyg ndadkfiper mlddefarin keypncwkpf gngkracigr pfawqeslla
  421 mvvlfqnfnf tmtdpnyale ikqtltikpd hfyinatlrh gmtptelehv lagngatsss
  481 thnikaaanl dakagsgkpm aifygsnsgt cealanrlas dapshgf sat tvgpldqakq
  541 nlpedrpvvi vtasyegqpp snaahfikwm edldgndmek vsyavfacgh hdwvetfhri
  601 pklvdstlek rggtrlvpmg sadaatsdmf sdfeawediv lwpglkekyk isdeesggqk
  661 gllvevstpr ktslrqdvee alvvaektlt ksgpakkhie iqlpsamtyk agdylailpl
  721 npkstvarvf rrfslawdsf lkiqsegptt 1ptnvaisaf dvfsayvels qpatkrnila
  781 laeatedkdt iqelerlagd ayqaeispkr vsvldllekf pavalpissy lamlppmrvr
  841 qysissspfa dpskltltys lldapslsgq grhvgvatnf lshltagdkl hvsvrassea
  901 fhlpsdaekt piicvaagtg laplrgfiqe raamlaagrt lapallffgc rnpeiddlya
  961 eeferwekmg avdvrraysr atdksegcky vqdrvyhdra dvfkvwdqga kvficgsrei
1021 gkavedvcvr laiekaqqng rdvteemara wfersrnerf atdvfd
SEQ ID NO: 37
CYPX
G. moniliformis
GenBank Accession No. AAG27132
>gi|11035011|gb|AAG27132.1|Fum6p [Fusarium verticillioides]
    1 MSATALFTRR SVSTSNPELR PIPGPKPLPL LGNLFDFDFD NLTKSLGELG KIHGPIYSIT
    61 FGASTEIMVT SREIAQELCD ETRFCKLPGG ALDVMKAVVG DGLFTAETSN PKWAIAHRII
  121 TPLFGAMRIR GMFDDMKDIC EQMCLRWARF GPDEPLNVCD NMTKLTLDTI ALCTIDYRFN
  181 SFYRENGAAH PFAEAVVDVM TESFDQSNLP DFVNNYVRFR AMAKFKRQAA ELRRQTEELI
  241 AARRQNPVDR DDLLNAMLSA KDPKTGEGLS PESIVDNLLT FLIAGHETTS SLLSFCFYYL
  301 LENPHVLRRV QQEVDTVVGS DTITVDHLSS MPYLEAVLRE TLRLRDPGPG FYVKPLKDEV
  361 VAGKYAVNKD QPLFIVFDSV HRDQSTYGAD ADEFRPERML KDGFDKLPPC AWKPFGNGVR
  421 ACVGRPFAMQ QAILAVAMVL HKFDLVKDES YTLKYHVTMT VRPVGFTMKV RLRQGQRATD
  481 LAMGLHRGHS QEASAAASPS RASLKRLSSD VNGDDTDHKS QIAVLYASNS GSCEALAYRL
  541 AAEATERGFG IRAVDVVNNA IDRIPVGSPV ILITASYNGE PADDAQEFVP WLKSLESGRL
  601 NGVKFAVFGN GHRDWANTLF AVPRLIDSEL ARCGAERVSL MGVSDTCDSS DPFSDFERWI
  661 DEKLFPELET PHGPGGVKNG DRAVPRQELQ VSLGQPPRIT MRKGYVRAIV TEARSLSSPG
  721 VPEKRHLELL LPKDFNYKAG DHVYILPRNS PRDVVRALSY FGLGEDTLIT IRNTARKLSL
  781 GLPLDTPITA TDLLGAYVEL GRTASLKNLW TLVDAAGHGS RAALLSLTEP ERFRAEVQDR
  841 HVSILDLLER FPDIDLSLSC FLPMLAQIRP RAYSFSSAPD WKPGHATLTY TVVDFATPAT
  901 QGINGSSKSK AVGDGTAVVQ RQGLASSYLS SLGPGTSLYV SLHRASPYFC LQKSTSLPVI
  961 MVGAGTGLAP FRAFLQERRM AAEGAKQRFG PALLFFGCRG PRLDSLYSVE LEAYETIGLV
1021 QVRRAYSRDP SAQDAQGCKY VTDRLGKCRD EVARLWMDGA QVLVCGGKKM ANDVLEVLGP
1081 MLLEIDQKRG ETTAKTVVEW RARLDKSRYV EEVYV
SEQ ID NO: 38
CYP505A7
G. zeae PH1
GenBank Accession No. EAA67736
>gi|42544893|gb|EAA67736.1|C505_FUSOX Bifunctional P-450:NADPH-P450
reductase (Fatty acid omega-hydroxylase) (P450foxy) [Gibberella zeae PH-1]
    1 MAESVPIPEP PGYPLIGNLG EFKTNPLNDL NRLADTYGPI FRLHLGSKTP TFVSSNAFIN
    61 EVCDEKRFKK TLKSVLSVVR EGVHDGLFTA FEDEPNWGKA HRILIPAFGP LSIRNMFPEM
  121 HEIANQLCMK LARHGPHTPV DASDNFTRLA LDTLALCAMD FRFNSYYKEE LHPFIEAMGD
  181 FLLESGNRNR RPAFAPNFLY RAANDKFYAD IALMKSVADE VVATRKQNPT DRKDLLAAML
  241 EGVDPQTGEK LSDDNITNQL ITFLIAGHET TSGTLSFAMY HLLKNPEAYN KLQKEIDEVI
  301 GRDPVTVEHL TKLPYLSAVL RETLRISSPI TGFGVEAIED TFLGGKYLIK KGETVLSVLS
  361 RGHVDPVVYG PDAEKFVPER MLDDEFARLN KEFPNCWKPF GNGKRACIGR PFAWQESLLA
  421 MALLFQNFNF TQTDPNYELQ IKQNLTIKPD NEFFNCTLRH GMTPTDLEGQ LAGKGATTSI
  481 ASHIKAPAAS KGAKASNGKP MAIYYGSNSG TCEALANRLA SDAAGHGFSA SVIGTLDQAK
  541 QNLPEDRPVV IVTASYEGQP PSNAAHFIKW MEDLAGNEME KVSYAVFGCG HHDWVDTFLR
  601 IPKLVDTTLE QRGGTRLVPM GSADAATSDM FSDFEAWEDT VLWPSLKEKY NVTDDEASGQ
  661 RGLLVEVTTP RKTTLRQDVE EALVVSEKTL TKTGPAKKHI EIQLPSGMTY KAGDYLAILP
  721 LNPRKTVSRV FRRFSLAWDS FLKIQSDGPT TLPINIAISA FDVFSAYVEL SQPATKRNIL
  781 ALSEATEDKA TIQELEKLAG DAYQEDVSAK KVSVLDLLEK YPAVALPISS YLAMLPPMRV
  841 RQYSISSSPF ADPSKLTLTY SLLDAPSLSG QGRHVGVATN FLSQLIAGDK LHISVRASSA
  901 AFHLPSDPET TPIICVAAGT GLAPFRGFIQ ERAAMLAAGR KLAPALLFFG CRDPENDDLY
  961 AEELARWEQM GAVDVRRAYS RATDKSEGCK YVQDRIYHDR ADVFKVWDQG AKVFICGSRE
1021 IGKAVEDICV RLAMERSEAT QEGKGATEEK AREWFERSRN ERFATDVFD
SEQ ID NO: 39
CYP505C2
G. zeae PHla
GenBank Accession No. EAA77183
>gi|42554340|gb|EAA77183.1|hypothetical protein FG07596.1 [Gibberella zeae
PH-1]
      1 MAIKDGGKKS GQIPGPKGLP VLGNLFDLDL SDSLTSLINI GQKYAPIFSL ELGGHREVMI
    61 CSRDLLDELC DETRFHKIVT GGVDKLRPLA GDGLFTAQHG NHDWGIAHRI LMPLFGPLKI
  121 REMFDDMQDV SEQLCLKWAR LGPSATIDVA NDFTRLTLDT IALCTMGYRF NSFYSNDKMH
  181 PFVDSMVAAL IDADKQSMFP DFIGACRVKA LSAFRKHAAI MKGTCNELIQ ERRKNPIEGT
  241 DLLTAMMEGK DPKTGEGMSD DLIVQNLITF LIAGHETTSG LLSFAFYYLL ENPHTLEKAR
  301 AEVDEVVGDQ ALNVDHLTKM PYVNMILRET LRLMPTAPGF FVTPHKDEII GGKYAVPANE
  361 SLFCFLHLIH RDPKVWGADA EEFRPERMAD EFFEALPKNA WKPFGNGMRG CIGREFAWQE
  421 AKLITVMILQ NFELSKADPS YKLKIKQSLT IKPDGFNMHA KLRNDRKVSG LFKAPSLSSQ
  481 QPSLSSRQSI NAINAKDLKP ISIFYGSNTG TCEALAQKLS ADCVASGFMP SKPLPLDMAT
  541 KNLSKDGPNI LLAASYDGRP SDNAEEFTKW AESLKPGELE GVQFAVFGCG HKDWVSTYFK
  601 IPKILDKCLA DAGAERLVEI GLTDASTGRL YSDFDDWENQ KLFTELSKRQ GVTPTDDSHL
  661 ELNVTVIQPQ NNDMGGNFKR AEVVENTLLT YPGVSRKHSL LLKLPKDMEY TPGDHVLVLP
  721 KNPPQLVEQA MSCFGVDSDT ALTISSKRPT FLPTDTPILI SSLLSSLVEL SQTVSRTSLK
  781 RLADFADDDD TKACVERIAG DDYTVEVEEQ RMSLLDILRK YPGINMPLST FLSMLPQMRP
  841 RTYSFASAPE WKQGHGMLLF SVVEAEEGTV SRPGGLATNY MAQLRQGDSI LVEPRPCRPE
  901 LRTTMMLPEP KVPIIMIAVG AGLAPFLGYL QKRFLQAQSQ RTALPPCTLL FGCRGAKMDD
  961 ICRAQLDEYS RAGVVSVHRA YSRDPDSQCK YVQGLVTKHS ETLAKQWAQG AIVMVCSGKK
1021 VSDGVMNVLS PILFAEEKRS GMTGADSVDV WRQNVPKERM ILEVFG
SEQ ID NO: 40
CYP505A5
M. grisea 70-15 syn
GenBank Accession No. XP 365223
>gi|145601517|ref|XP_365223.21 hypothetical protein MGG 01925 [Magnaporthe
oryzae 70-15]
      1 MFFLSSSLAY MAATQSRDWA SFGVSLPSTA LGRHLQAAMP FLSEENHKSQ GTVLIPDAQG
    61 PIPFLGSVPL VDPELPSQSL QRLARQYGEI YRFVIPGRQS PILVSTHALV NELCDEKRFK
  121 KKVAAALLGL REAIHDGLFT AHNDEPNWGI AHRILMPAFG PMAIKGMFDE MHDVASQMIL
  181 KWARHGSTTP IMVSDDFTRL TLDTIALCSM GYRFNSFYHD SMHEFIEAMT CWMKESGNKT
  241 RRLLPDVFYR TTDKKWHDDA EILRRTADEV LKARKENPSG RKDLLTAMIE GVDPKTGGKL
  301 SDSSIIDNLI TFLIAGHETT SGMLSFAFYL LLKNPTAYRK AQQEIDDLCG REPITVEHLS
  361 KMPYITAVLR ETLRLYSTIP AFVVEAIEDT VVGGKYAIPK NHPIFLMIAE SHRDPKVYGD
  421 DAQEFEPERM LDGQFERRNR EFPNSWKPFG NGMRGCIGRA FAWQEALLIT AMLLQNFNFV
  481 MHDPAYQLSI KENLTLKPDN FYMRAILRHG MSPTELERSI SGVAPTGNKT PPRNATRTSS
  541 PDPEDGGIPM SIYYGSNSGT CESLAHKLAV DASAQGFKAE TVDVLDAANQ KLPAGNRGPV
  601 VLITASYEGL PPDNAKHFVE WLENLKGGDE LVDTSYAVFG CGHQDWTKTF HRIPKLVDEK
  661 LAEHGAVRLA PLGLSNAAHG DMFVDFETWE FETLWPALAD RYKTGAGRQD AAATDLTAAL
  721 SQLSVEVSHP RAADLRQDVG EAVVVAARDL TAPGAPPKRH MEIRLPKTGG RVHYSAGDYL
  781 AVLPVNPKST VERAMRRFGL AWDAHVTIRS GGRTTLPTGA PVSAREVLSS YVELTQPATK
  841 RGIAVLAGAV TGGPAAEQEQ AKAALLDLAG DSYALEVSAK RVGVLDLLER FPACAVPFGT
  901 FLALLPPMRV RQYSISSSPL WNDEHATLTY SVLSAPSLAD PARTHVGVAS SYLAGLGEGD
  961 HLHVALRPSH VAFRLPSPET PVVCVCAGSG MAPFRAFAQE RAALVGAGRK VAPLLLFFGC
1021 REPGVDDLYR EELEGWEAKG VLSVRRAYSR RTEQSEGCRY VQDRLLKNRA EVKSLWSQDA
1081 KVFVCGSREV AEGVKEAMFK VVAGKEGSSE EVQAWYEEVR NVRYASDIFD
SEQ ID NO: 41
CYP505A2
N. crassa 0R74 A
GenBank Accession No. XP 961848
>gi|85104987|ref|XP_961848.1|bifunctional P-450:NADPH-P450 reductase
[Neurospora crassa OR74A]
      1 MSSDETPQTI PIPGPPGLPL VGNSFDIDTE FPLGSMLNFA DQYGEIFRLN FPGRNTVFVT
    61 SQALVHELCD EKRFQKTVNS ALHEIRHGIH DGLFTARNDE PNWGIAHRIL MPAFGPMAIQ
  121 NMFPEMHEIA SQLALKWARH GPNQSIKVTD DFTRLTLDTI ALCSMDYRFN SYYHDDMHPF
  181 IDAMASFLVE SGNRSRRPAL PAFMYSKVDR KFYDDIRVLR ETAEGVLKSR KEHPSERKDL
  241 LTAMLDGVDP KTGGKLSDDS IIDNLITFLI AGHETTSGLL SFAFVQLLKN PETYRKAQKE
  301 VDDVCGKGPI KLEHMNKLHY IAAVLRETLR LCPTIPVIGV ESKEDTVIGG KYEVSKGQPF
  361 ALLFAKSHVD PAVYGDTAND FDPERMLDEN FERLNKEFPD CWKPFGNGMR ACIGRPFAWQ
  421 EALLVMAVCL QNFNFMPEDP NYTLQYKQTL TTKPKGFYMR AMLRDGMSAL DLERRLKGEL
  481 VAPKPTAQGP VSGQPKKSGE GKPISIYYGS NTGTCETFAQ RLASDAEAHG FTATIIDSLD
  541 AANQNLPKDR PVVFITASYE GQPPDNAALF VGWLESLTGN ELEGVQYAVF GCGHHDWAQT
  601 FHRIPKLVDN TVSERGGDRI CSLGLADAGK GEMFTEFEQW EDEVFWPAME EKYEVSRKED
  661 DNEALLQSGL TVNFSKPRSS TLRQDVQEAV VVDAKTITAP GAPPKRHIEV QLSSDSGAYR
  721 SGDYLAVLPI NPKETVNRVM RRFQLAWDTN ITIEASRQTT ILPTGVPMPV HDVLGAYVEL
  781 SQPATKKNIL ALAEAADNAE TKATLRQLAG PEYTEKITSR RVSILDLLEQ FPSIPLPFSS
  841 FLSLLPPMRV RQYSISSSPL WNPSHVTLTY SLLESPSLSN PDKKHVGVAT SYLASLEAGD
  901 KLNVSIRPSH KAFHLPVDAD KTPLIMIAAG SGLAPFRGFV QERAAQIAAG RSLAPAMLFY
  961 GCRHPEQDDL YRDEFDKWES IGAVSVRRAF SRCPESQETK GCKYVGDRLW EDREEVTGLW
1021 DRGAKVYVCG SREVGESVKK VVVRIALERQ KMIVEAREKG ELDSLPEGIV EGLKLKGLTV
1081 EDVEVSEERA LKWFEGIRNE RYATDVFD
SEQ ID NO: 42
CYP97C
Oryza sativa
GenBank Accession No. ABB47954
>gi|78708979|gb|ABB47954.1|Cytochrome P450 family protein, expressed
[Oryza sativa Japonica Group]
      1 MAAAAAAAVP CVPFLCPPPP PLVSPRLRRG HVRLRLRPPR SSGGGGGGGA GGDEPPITTS
    61 WVSPDWLTAL SRSVATRLGG GDDSGIPVAS AKLDDVRDLL GGALFLPLFK WFREEGPVYR
  121 LAAGPRDLVV VSDPAVARHV LRGYGSRYEK GLVAEVSEFL FGSGFAIAEG ALWTVRRRSV
  181 VPSLHKRFLS VMVDRVFCKC AERLVEKLET SALSGKPVNM EARFSQMTLD VIGLSLFNYN
  241 FDSLTSDSPV IDAVYTALKE AELRSTDLLP YWKIDLLCKI VPRQIKAEKA VNIIRNTVED
  301 LITKCKKIVD AENEQIEGEE YVNEADPSIL RFLLASREEV TSVQLRDDLL SMLVAGHETT
  361 GSVLTWTIYL LSKDPAALRR AQAEVDRVLQ GRLPRYEDLK ELKYLMRCIN ESMRLYPHPP
  421 VLIRRAIVDD VLPGNYKIKA GQDIMISVYN IHRSPEVWDR ADDFIPERFD LEGPVPNETN
  481 TEYRFIPFSG GPRKCVGDQF ALLEAIVALA VVLQKMDIEL VPDQKINMTT GATIHTTNGL
  541 YMNVSLRKVD REPDFALSGS R
SEQ ID NO: 43
Chimeric heme enzyme C2G9
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTESQRAMKDYHEKMVDIAVQLIQKWARLNPNEAVDVPGDMTRL
TLDTIGLCGFNYRFNSYYRETPHPFINSMVRALDEAMHQMQRLDVQDKLMVRTKRQFRYDIQTMFSLVDRMIAER
KANPDENIKDLLSLMLYAKDPVTGETLDDENIRYQIITFLIAGHETTSGLLSFALYELVKNPHVLQKAAEEAARV
LVDPVPSYKQVKQLKYVGMVLNEALRLWPTAPAFSLYAKEDTVLGGEYPISKGQPVTVLIPKLHRDQNAWGPDAE
DERPERFEDPSSIPHHAYKPFGNGQRACIGMQFALHEATLVLGMILKYFTLIDHENYELDIKQTLTLKPGDFHIS
VQSRHQEAIHADVQAAE
SEQ ID NO: 44
Chimeric heme enzyme X7
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTESQRAMKDYHEKMVDIATQLIQKWSRLNPNEEIDVADDMTRL
TLDTIGLCGENYRENSFYRDQPHPFITSMVRALDEAMNKLQRANPDDPAYDENKRQFQEDIKVMNDLVDSIIAER
RANGDQDEKDLLARMLNVEDPETGEKLDDENIRFQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DERPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 45
Chimeric heme enzyme X7-12
MTIKEMPQPKTFGELKNLPLLNTDKPVQALMKIADELGEIFKFEAPGRVTRYLSSQRLIKEACDEERFDKSIEGA
LEKVRAFSGDGLATSWTHEPNWRKAHNILMPTESQRAMKDYHEKMVDIAVQLVQKWERLNADEHIEVPEDMTRLT
LDTIGLCGENYRENSFYRDQPHPFITSMVRALDEAMNKLQRANPDDPAYDENKRQFQEDIKVMNDLVDSIIAERR
ANGDQDEKDLLARMLNVEDPETGEKLDDENIRFQIITFLIAGHETTSGLLSFAIYCLLTHPEKLKKAQEEADRVL
TDDTPEYKQIQQLKYIRMVLNETLRLYPTAPAFSLYAKEDTVLGGEYPISKGQPVTVLIPKLHRDQNAWGPDAED
FRPERFEDPSSIPHHAYKPFGNGQRACIGMQFALQEATMVLGLVLKHFELINHTGYELKIKEALTIKPDDFKITV
KPRKTAAINVQRKEQA
SEQ ID NO: 46
Chimeric heme enzyme C2E6
MTIKEMPQPKTFGELKNLPLLNTDKPVQALMKIADELGEIFKFEAPGRVTRYLSSQRLIKEACDESRFDKNLSQA
LKEVRDFAGDGLFTSWTHEKNWKKAHNILLPSFSQQAMKGYHAMMVDIAVQLVQKWERLNADEHIEVPEDMTRLT
LDTIGLCGENYRENSFYRDQPHPFITSMVRALDEAMNKLQRANPDDPAYDENKRQFQEDIKVMNDLVDRMIAERK
ANPDENIKDLLSLMLYAKDPVTGETLDDENIRYQIITFLIAGHETTSGLLSFAIYCLLTHPEKLKKAQEEADRVL
TDDTPEYKQIQQLKYIRMVLNETLRLYPTAPAFSLYAKEDTVLGGEYPLEKGDELMVLIPQLHRDKTIWGDDVEE
FRPERFENPSAIPQHAFKPFGNGQRACIGQQFALHEATLVLGMMLKHEDFEDHTNYELDIKETLTLKPEGFVVKA
KSKKIPLGGIPSPST
SEQ ID NO: 47
Chimeric heme enzyme X7-9
MKQASAIPQPKTYGPLKNLPHLEKEQLSQSLWRIADELGPIFRFDFPGVSSVFVSGHNLVAEVCDEERFDKSIEG
ALEKVRAFSGDGLATSWTHEPNWRKAHNILMPTFSQRAMKDYHEKMVDIATQLIQKWSRLNPNEEIDVADDMTRL
TLDTIGLCGFNYRFNSFYRDQPHPFITSMVRALDEAMNKLQRANPDDPAYDENKRQFQEDIKVMNDLVDSIIAER
RANGDQDEKDLLARMLNVEDPETGEKLDDENIRFQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DFRPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 48
Chimeric heme enzyme C2B12
MKQASAIPQPKTYGPLKNLPHLEKEQLSQSLWRIADELGPIFRFDFPGVSSVFVSGHNLVAEVCDEERFDKSIEG
ALEKVRAFSGDGLATSWTHEPNWRKAHNILMPTESQRAMKDYHEKMVDIATQLIQKWSRLNPNEEIDVADDMTRL
TLDTIGLCGENYRENSFYRDQPHPFITSMVRALDEAMNKLQRANPDDPAYDENKRQFQEDIKVMNDLVDRMIAER
KANPDENIKDLLSLMLYAKDPVTGETLDDENIRYQIITFLIAGHETTSGLLSFATYFLLKHPDKLKKAYEEVDRV
LTDAAPTYKQVLELTYIRMILNESLRLWPTAPAFSLYAKEDTVLGGEYPISKGQPVTVLIPKLHRDQNAWGPDAE
DERPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 49
Chimeric heme enzyme T5P234
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTESQRAMKDYHEKMVDIATQLIQKWSRLNPNEEIDVADDMTRL
TLDTIGLCGENYRENSFYRDQPHPFITSMVRALDEAMNKLQRANPDDPAYDENKRQFQEDIKVMNDLVDRMIAER
KANPDENIKDLLSLMLYAKDPVTGETLDDENIRYQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DERPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 50
WT-Ax A (heme)
TIKEMPQPKTFGELKNLPLLNTDKPVQALMKIADELGEIFKFEAPGRVTRYLSSQRLIKEACDESRFDKNLSQAL
KEVRDFAGDGLFTSWTHEKNWKKAHNILLPSFSQQAMKGYHAMMVDIAVQLVQKWERLNADEHIEVPEDMTRLTL
DTIGLCGENYRENSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYELVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAAIGQQFALHEATLVLGMMLKHEDFEDHTNYELDIKETLSLKPKGFVVKAKS
KKIPLGGIPSPST
SEQ ID NO: 51
WT-AxD (heme)
TIKEMPQPKTFGELKNLPLLNTDKPVQALMKIADELGEIFKFEAPGRVTRYLSSQRLIKEACDESRFDKNLSQAL
KEVRDFAGDGLFTSWTHEKNWKKAHNILLPSFSQQAMKGYHAMMVDIAVQLVQKWERLNADEHIEVPEDMTRLTL
DTIGLCGENYRENSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYELVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRADIGQQFALHEATLVLGMMLKHEDFEDHTNYELDIKETLSLKPKGFVVKAKS
KKIPLGGIPSPST
SEQ ID NO: 52
WT-AxH (heme)
TIKEMPQPKTFGELKNLPLLNTDKPVQALMKIADELGEIFKFEAPGRVTRYLSSQRLIKEACDESRFDKNLSQAL
KEVRDFAGDGLFTSWTHEKNWKKAHNILLPSFSQQAMKGYHAMMVDIAVQLVQKWERLNADEHIEVPEDMTRLTL
DTIGLCGENYRENSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYELVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAHIGQQFALHEATLVLGMMLKHEDFEDHTNYELDIKETLSLKPKGFVVKAKS
KKIPLGGIPSPST
SEQ ID NO: 53
WT-AxK (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAKIGQQFALHEATLVLGMMLKHFDFEDHTNYELDIKETLSLKPKGFVVKAKS
KKIPLGGIPSPST
SEQ ID NO: 54
WT-AxM (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAMIGQQFALHEATLVLGMMLKHFDFEDHTNYELDIKETLSLKPKGFVVKAKS
KKIPLGGIPSPST
SEQ ID NO: 55
WT-AxN (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRANIGQQFALHEATLVLGMMLKHFDFEDHTNYELDIKETLSLKPKGFVVKAKS
KKIPLGGIPSPST
SEQ ID NO: 56
BM3-CIS-T4385-AxA
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAAIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 57
BM3-CIS-T4385-AxD
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRADIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 58
BM3-CIS-T438S-AxM
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAMIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 59
BM3-CIS-T4385-AxY
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAYIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 60
BM3-CIS-T4385-AxT
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRATIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 61
M. infernorum Hemoglobin I
      1 MIDQKEKELI KESWKRIEPN KNEIGLLFYA NLFKEEPTVS VLFQNPISSQ
    51 SRKLMQVLGI LVQGIDNLEG LIPTLQDLGR RHKQYGVVDS HYPLVGDCLL
  101 KSIQEYLGQG FTEEAKAAWT KVYGIAAQVM TAE
SEQ ID NO: 62
Bacillus subtilis truncated hemoglobin
      1 MGQSFNAPYE AIGEELLSQL VDTFYERVAS HPLLKPIFPS DLTETARKQK
    51 QFLTQYLGGP PLYTEEHGHP MLRARHLPFP ITNERADAWL SCMKDAMDHV
  101 GLEGEIREFL FGRLELTARH MVNQTEAEDR SS
```

What is claimed is:

1. A reaction mixture for producing a cyclopropanation product of Formula A: 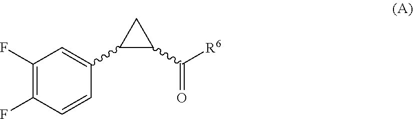
wherein R⁶ is selected from the group consisting of C₁₋₁₈ alkyl, C₁₋₁₈ alkenyl, C₁₋₁₈ alkynyl, C₁₋₁₈ alkoxy, C₁₋₁₈ alkenyloxy, C₁₋₁₈ alkynyloxy; and
the reaction mixture comprises an olefinic substrate, a carbene precursor, and a heme enzyme.

2. The reaction mixture of claim 1, wherein R⁶ is C₁₋₁₈ alkoxy and the carbene precursor is a diazoester.

3. The reaction mixture of claim 2, wherein the cyclopropanation product is a compound of Formula XVII: 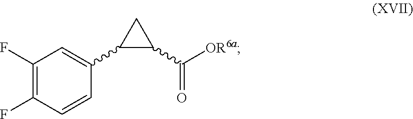
the olefinic substrate is a compound of Formula V 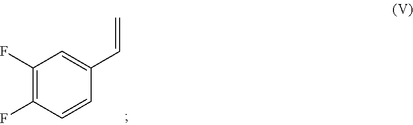
and
the carbene precursor is a compound of Formula XVI, 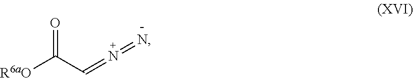
wherein R^{6a} is C₁₋₁₈ alkyl.

4. The reaction mixture of claim 3, wherein the cyclopropanation product is a compound according to Formula XVIIa:

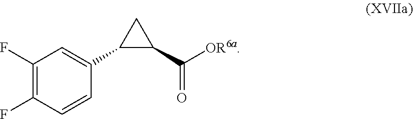

5. The reaction mixture of claim 3, wherein the cyclopropanation product is a compound according to Formula VIIa:

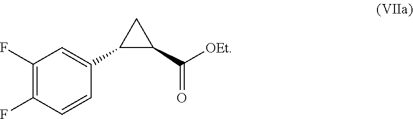 [SMILES: CCOC(=O)[C@@H]1C[C@H]1c1ccc(F)c(F)c1]

6. The reaction mixture of claim 1, wherein R⁶ is selected from the group consisting of C₁₋₁₈ alkyl, C₁₋₁₈ alkenyl, and C₁₋₁₈ alkynyl and the carbene precursor is a diazoketone.

7. The reaction mixture of claim 6, wherein the cyclopropanation product is a compound of Formula XXVII: 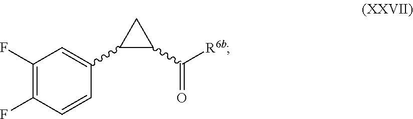
the olefinic substrate is a compound of Formula V: 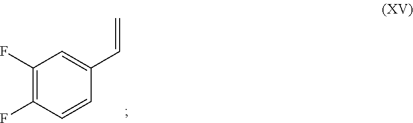
and
the carbene precursor is a compound of Formula XXVI: 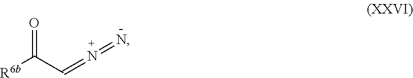
wherein R^{6b} is C₁₋₁₈ alkyl.

8. The reaction mixture of claim 7, wherein the cyclopropanation product is a compound according to Formula XXVIIa:

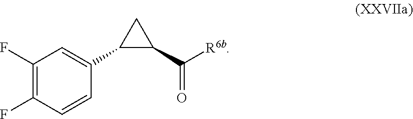

9. The reaction mixture of claim 8, wherein R^{6b} is methyl.

10. The reaction mixture of any one of the preceding claims, wherein the heme enzyme comprises a mutation at the axial position of the heme coordination site.

11. The reaction mixture of any one of the preceding claims, wherein the heme enzyme is a cytochrome P450 enzyme or a variant or homolog thereof.

12. The reaction mixture of claim 11, wherein the cytochrome P450 enzyme is a P450 BM3 enzyme or a variant or homolog thereof.

13. The reaction mixture of claim 11, wherein the cytochrome P450 enzyme is a CYP119 enzyme or a variant or homolog thereof.

14. The reaction mixture of claim 11, wherein the cytochrome P450 enzyme is a CYP119 variant or homolog encoding a mutation at position H315 to any other amino acid, for example alanine, cysteine, aspartate, glutamate, phenylalanine, glycine, isoleucine, lysine, leucine, methionine, asparagine, proline, glutamine, arginine, serine, threonine, valine, tryptophan, or tyrosine.

15. The reaction mixture of any one of the preceding claims, wherein the heme enzyme is a cytochrome c or a variant or homolog thereof.

16. The reaction mixture of claim 1, wherein the heme enzyme is a globin or a variant or homolog thereof.

17. The reaction mixture of any one of claims 2-10, wherein the heme enzyme is a globin or a variant or homolog thereof.

18. The reaction mixture of claim 16, wherein the heme enzyme is a myoglobin or a variant or homolog thereof.

19. The reaction mixture of claim 16, wherein the globin is M. infernorum hemoglobin according to SEQ ID NO: 61 or a variant or homolog thereof.

20. The reaction mixture of claim 19, wherein the M. infernorum hemoglobin variant or homolog comprises one or more mutations of amino acid residues selected from the group consisting of F28, Y29, L32, L54, and V95.

21. The reaction mixture of claim 19, wherein the M. infernorum hemoglobin variant or homolog comprises one or more mutations selected from the group consisting of F28S, Y29A, L32A, L32C, L32T, L54S, and V95F.

22. The reaction mixture of claim 21, wherein the M. infernorum variant or homolog comprises a V95F mutation.

23. The reaction mixture of claim 16, wherein the globin is B. subtilis truncated hemoglobin according to SEQ ID NO: 62 or a variant or homolog thereof.

24. The reaction mixture of claim 23, where the B. subtilis hemoglobin variant or homolog comprises one or more mutations of amino acid residues selected from the group consisting of T45 and Q49.

25. The reaction mixture of claim 24, where the B. subtilis hemoglobin variant or homolog comprises a T45 mutation and a Q49 mutation.

26. The reaction mixture of claim 23, where the B. subtilis hemoglobin variant or homolog comprises one or more mutations selected from the group consisting of T45L, T45F, T45A, Q49L, Q49F, and Q49A.

27. The reaction mixture of claim 26, where the B. subtilis hemoglobin variant or homolog comprises a first mutation selected from the group consisting of T45L, T45F, and T45A, and a second mutation selected from the group consisting of Q49L, Q49F, and Q49A.

28. The reaction mixture of any one of the preceding claims, wherein the cyclopropanation product is produced in vitro.

29. The reaction mixture of any one of the preceding claims, wherein the reaction mixture further comprises a reducing agent.

30. The reaction mixture of any one of the preceding claims, wherein the heme enzyme is localized within a whole cell and the cyclopropanation product is produced in vivo.

31. The reaction mixture of any one of the preceding claims, wherein the cyclopropanation product is produced under anaerobic conditions.

32. A method for producing a cyclopropanation product of Formula A: 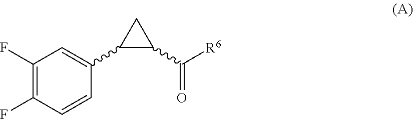
wherein R⁶ is selected from the group consisting of C₁₋₁₈ alkyl, C₁₋₁₈ alkenyl, C₁₋₁₈ alkynyl, C₁₋₁₈ alkoxy, C₁₋₁₈ alkenyloxy, C₁₋₁₈ alkynyloxy;
the method comprising forming a reaction mixture containing an olefinic substrate, a carbene precursor, and a heme enzyme under conditions sufficient to form the cyclopropanation product.

33. The method of claim 32, wherein R⁶ is C₁₋₁₈ alkoxy and the carbene precursor is a diazoester.

34. The method of claim 33, wherein the cyclopropanation product is a compound of Formula XVII: 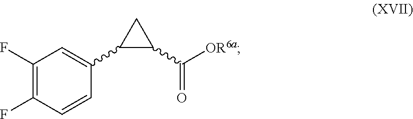
the olefinic substrate is a compound of Formula V 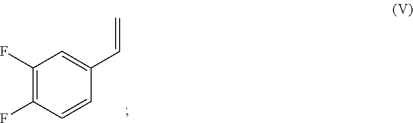
and
the carbene precursor is a compound of Formula XVI, 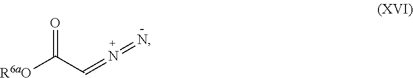
wherein R^{6a} is C₁₋₁₈ alkyl.

35. The method of claim 34, wherein the cyclopropanation product is a compound according to Formula XVIIa:

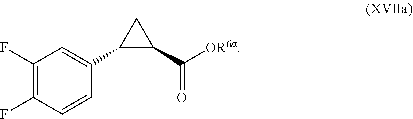

36. The method of claim 34, wherein the cyclopropanation product is a compound according to Formula VIIa:

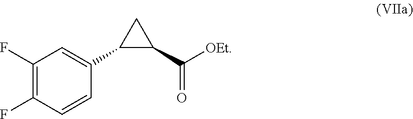 [SMILES: CCOC(=O)[C@@H]1C[C@H]1c1ccc(F)c(F)c1]

37. The method of claim 32, wherein R⁶ is selected from the group consisting of C₁₋₁₈ alkyl, C₁₋₁₈ alkenyl, and C₁₋₁₈ alkynyl and the carbene precursor is a diazoketone.

38. The method of claim 37, wherein the cyclopropanation product is a compound of Formula XXVII: 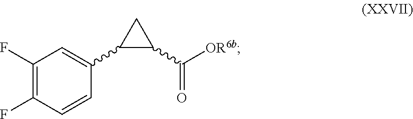
the olefinic substrate is a compound of Formula V: 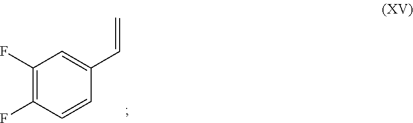
and
the carbene precursor is a compound of Formula XXVI: 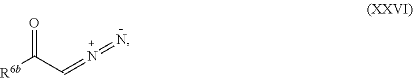
wherein R^{6b} is C₁₋₁₈ alkyl.

39. The method of claim 38, wherein the cyclopropanation product is a compound according to Formula XXVIIa:

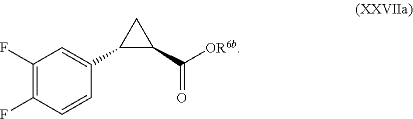

40. The method of claim 39, wherein R^{6b} is methyl.

41. The method of any one of claims 32-39, wherein the heme enzyme comprises a mutation at the axial position of the heme coordination site.

42. The method of any one of claims 32-41, wherein the heme enzyme is a cytochrome P450 enzyme or a variant thereof.

43. The method of claim 42, wherein the cytochrome P450 enzyme is a P450 BM3 enzyme or a variant thereof.

44. The method of claim 42, wherein the cytochrome P450 enzyme is a CYP119 enzyme or a variant thereof.

45. The method of claim 42, wherein the cytochrome P450 enzyme is a CYP119 variant encoding a mutation at position H315 to any other amino acid, for example alanine, cysteine, aspartate, glutamate, phenylalanine, glycine, isoleucine, lysine, leucine, methionine, asparagine, proline, glutamine, arginine, serine, threonine, valine, tryptophan, or tyrosine.

46. The method of any one of claims 32-41, wherein the heme enzyme is a cytochrome c or a variant thereof.

47. The method of claim 32, wherein the heme enzyme is a globin or a variant thereof.

48. The method of any one of claims 33-41, wherein the heme enzyme is a globin or a variant thereof.

49. The method of claim 47, wherein the heme enzyme is a myoglobin or a variant thereof.

50. The method of claim 47, wherein the globin is M. infernorum hemoglobin according to SEQ ID NO: 61 or a variant thereof.

51. The method of claim 50, wherein the M. infernorum hemoglobin variant comprises one or more mutations of amino acid residues selected from the group consisting of F28, Y29, L32, L54, and V95.

52. The method of claim 50, wherein the M. infernorum hemoglobin variant comprises one or more mutations selected from the group consisting of F28S, Y29A, L32A, L32C, L32T, L54S, and V95F.

53. The method of claim 50, wherein the M. infernorum variant comprises a V95F mutation.

54. The method of claim 47, wherein the globin is B. subtilis truncated hemoglobin according to SEQ ID NO: 62 or a variant thereof.

55. The method of claim 54, where the B. subtilis hemoglobin variant comprises one or more mutations of amino acid residues selected from the group consisting of T45 and Q49.

56. The method of claim 55, where the B. subtilis hemoglobin variant comprises a T45 mutation and a Q49 mutation.

57. The method of claim 54, where the B. subtilis hemoglobin variant comprises one or more mutations selected from the group consisting of T45L, T45F, T45A, Q49L, Q49F, and Q49A.

58. The method of claim 54, where the B. subtilis hemoglobin variant comprises a first mutation selected from the group consisting of T45L, T45F, and T45A, and a second mutation selected from the group consisting of Q49L, Q49F, and Q49A.

59. The method of any one of claims 32-58, wherein the cyclopropanation product is produced in vitro.

60. The method of any one of claims 32-59, wherein the reaction mixture further comprises a reducing agent.

61. The method of any one of claims 32-60, wherein the heme enzyme is localized within a whole cell and the cyclopropanation product is produced in vivo.

62. The method of any one of claims 32-61, wherein the cyclopropanation product is produced under anaerobic conditions.

63. The method of any one of claims 32-62, further comprising converting the cyclopropanation product to ticagrelor.
